(19) United States
(12) Patent Application Publication
(10) Pub. No.: US 2010/0056525 A1
(43) Pub. Date: Mar. 4, 2010

(54) 5- AND 6- SUBSTITUTED BENZIMIDAZOLE THIOPHENE COMPOUNDS

(76) Inventors: Tara Renae Rheault, Durham, NC (US); Mui Cheung, Durham, NC (US); Jennifer G. Badiang Alberti, Durham, NC (US); Kelly Horne Donaldson, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/447,909

(22) PCT Filed: Oct. 30, 2007
(86) PCT No.: PCT/US07/82951

§ 371 Date: Apr. 30, 2009

Related U.S. Application Data
(60) Provisional application No. 60/863,662, filed on Oct. 31, 2006.

(51) Int. Cl. A61K 31/5377; C07D 235/04; C07D 409/14; C07D 413/14; A61K 31/4184; A61K 31/497; A61P 35/00

(52) U.S. Cl. 514/235.8; 548/304.7; 544/364; 544/131; 514/394; 514/253.09

(57) ABSTRACT

The present invention provides 5- and 6-substituted benzimidazole thiophene compounds pharmaceutical compositions containing the same, processes for preparing the same and their use as pharmaceutical agents.

### BACKGROUND OF THE INVENTION

The present invention relates to novel benzimidazole thiophene compounds, pharmaceutical formulations comprising these compounds, and the use of these compounds in therapy.

Polo-like kinases (“PLK”) are evolutionarily conserved serine/threonine kinases that play critical roles in regulating processes in the cell cycle. PLK plays a role in the entry into and the exit from mitosis in diverse organisms from yeast to mammalian cells. PLK includes PLK1, PLK2, PLK3 and PLK4.

Overexpression of PLK1 appears to be strongly associated with neoplastic cells (including cancers). A published study has shown high levels of PLK1 RNA expression in >80% of lung and breast tumors, with little to no expression in adjacent normal tissue. Several studies have shown correlations between PLK expression, histological grade, and prognosis in several types of cancer. Significant correlations were found between percentages of PLK-positive cells and histological grade of ovarian and endometrial cancer (P<0.001). These studies noted that PLK is strongly expressed in invading endometrial carcinoma cells and that this could reflect the degree of malignancy and proliferation in endometrial carcinoma. Using RT-PCR analysis, PLK overexpression was detected in 97% of esophageal carcinomas and 73% of gastric carcinomas as compared to the corresponding normal tissues. Further, patients with high levels of PLK overexpression in esophageal carcinoma represented a significantly poorer prognosis group than those with low levels of PLK overexpression. In head and neck cancers, elevated mRNA expression of PLK1 was observed in most tumors; a Kaplan-Meier analysis showed that those patients with moderate levels of PLK1 expression survived longer than those with high levels of PLK1 expression. Analysis of patients with non-small cell lung carcinoma showed similar outcomes related to PLK1 expression.

PCT Publication No. WO2004/014899 to SmithKline Beecham discloses novel benzimidazole thiophene compounds of formula (I):

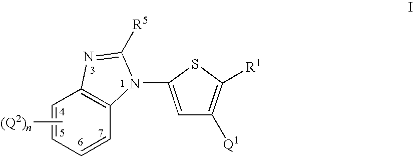

wherein:
R¹ is selected from the group consisting of H, alkyl, alkenyl, alkynyl, —C(O)R⁷, —CO₂R⁷, —C(O)NR⁷R³, —C(O)N(R⁷)OR³, —C(O)N(R⁷)—R²—OR⁸, —C(O)N(R⁷)-Ph, —C(O)N(R⁷)—R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷)NR⁷R⁸, —C(═NR⁷)N(R⁸)_Ph, —C(═NR⁷)N(R⁸)—R²-Ph, —R²—NR⁷R³, —CN, —OR⁷, —S(O)_{f}R⁷, —S(O)₂NR⁷R³, —S(O)₂N(R⁷)-Ph, —S(O)₂N(R⁷)—R²-Ph, —NR⁷R⁸, N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
Q¹ is a group of formula: —(R²)ₐ—(Y¹)_{b}—(R²)_{c}—R³
a, b and c are the same or different and are each independently 0 or 1 and at least one of a or b is 1;
n is 0, 1, 2, 3 or 4;
Q² is a group of formula: —(R²)ₐₐ—(Y²)_{bb}—(R²)_{cc}—R⁴
   or two adjacent Q² groups are selected from the group consisting of alkyl, alkenyl, —OR⁷, —S(O)_{f}R⁷ and —NR⁷R³ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y¹ and Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R³ and R⁴ is the same or different and is each independently selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R³, —CR⁷═N—OR⁷, —OR⁷, —S(O)_{f}R⁷, —S(O)₂NR⁷R³, —NR⁷R³, —N(R⁷)C(O)R⁸, —N(R⁷)S(O)₂R⁸, —NO₂, —CN, —N₃ and a group of formula (ii):

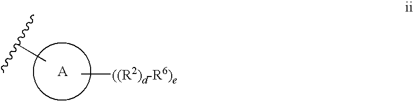

wherein:
Ring A is selected from the group consisting of C₅₋₁₀cycloalkyl, C₅₋₁₀cycyloalkenyl, aryl, 5-10 membered heterocycle having 1, 2 or 3 heteroatoms selected from N, O and 5 and 5-10 membered heteroaryl having 1, 2 or 3 heteroatoms selected from N, O and S
each d is 0 or 1;
e is 0, 1, 2, 3 or 4;
each R⁶ is the same or different and is independently selected from the group consisting of H, halo, alkyl, alkenyl, alkynyl, cycloalkyl, cycloalkenyl, Ph, Het, —CH(OH)—R²—OH, —C(O)R⁷, —CO₂R⁷, —CO₂—R₂-Ph, —CO₂—R²—Het, —C(O)NR⁷R⁸, —C(O)N(R⁷)C(O)R⁷, —C(O)N(R⁷)CO₂R⁷, —C(O)N(R⁷)C(O)NR⁷R⁸, —C(O)N(R⁷)S(O)₂R⁷, —C(S)R⁷, —C(S)NR⁷R³, —C(═NR⁷)R³, —C(═NR⁷)NR⁷R³, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷, R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²—Het, —N(R⁷)S(O)₂R⁸, —N(R⁷)—R²—S(O)₂R⁸, —NO₂, —CN and —N₃;
wherein when Q¹ is defined where b is 1 and c is O, R³ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R³, —CR⁷═N—OR⁷, —OR⁷, —S(O)_{f}R⁷, —S(O)₂NR⁷R³, —NR⁷R³, —N(R⁷)C(O)R⁸, —N(R⁷)S(O)₂R⁸, —NO₂, —CN or —N₃;
wherein when Q² is defined where bb is 1 and cc is O, R⁴ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R³, —CR⁷═N—OR⁷, —OR⁷, —S(O)_{f}R⁷, —S(O)₂NR⁷R³, —NR⁷R³, —N(R⁷)C(O)R⁸, —N(R⁷)S(O)₂R⁸, —NO₂, —CN or —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R³ are the same or different and are each independently selected from the group consisting of H, alkyl, alkenyl, alkynyl, cycloalkyl and cycloalkenyl;
wherein when R¹ is —CO₂CH₃ and n is 0, Q¹ is not —OH;
   or a pharmaceutically acceptable salt thereof.

Also disclosed are pharmaceutical compositions containing these compounds, processes for their preparation and methods for treatment of conditions mediated by PLK using these compounds.

### BRIEF SUMMARY OF THE INVENTION

According to a first aspect of the invention there is provided compounds of formula (I):

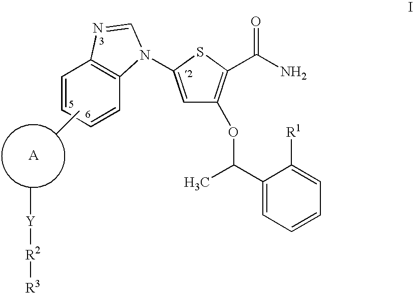

wherein
R¹ is F, Cl, CH₃ or CF₃;
Ring A is selected from phenyl and 5-6 membered heteroaryl having 1 or 2 heteroatoms selected from N, O and S;
Y is selected from —O—, —N(R⁴)—, —N(R⁴)—C(O)— and —N(R⁴)—R²—OC(O)—;
R² is linear or branched C₁₋₄alkylene optionally substituted once by —OH;
R³ is selected from —OR⁴, —N(R⁴)₂ and Het;
each R⁴ is the same or different and is independently H or C₁₋₄alkyl;
Het is a 5-6 member heterocycle having 1 or 2 heteroatoms selected from N, O and S and optionally substituted by 1 or 2 substituents selected from halo, C₁₋₄alkyl, oxo, —OR⁴, —C(O)R⁴, —C(O)₂R⁴, —SO₂R⁴— and CN;
   and pharmaceutically acceptable salts thereof.

In second aspect, there is provided an enantiomerically enriched compound of formula (I) having the stereochemistry depicted in formula (I-1).

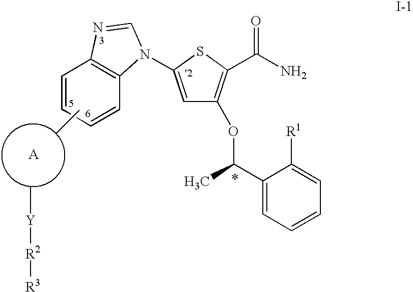

wherein * indicates the chiral carbon and all variables are as defined above.

In a fourth aspect of the invention there is provided a pharmaceutical composition comprising a compound of formula (I) or (I-1) or a pharmaceutically acceptable salt thereof. In one embodiment, the pharmaceutical composition further comprises a pharmaceutically acceptable carrier, diluent or excipient.

In a fifth aspect of the invention, there is provided a method for treating a condition mediated by PLK in a mammal in need thereof. The method comprises administering to the mammal a therapeutically effective amount of a compound of formula (I) or (I-1) or a pharmaceutically acceptable salt thereof.

In a sixth aspect of the invention, there is provided a method for treating a susceptible neoplasm in a mammal in need thereof. The method comprises administering to the mammal a therapeutically effective amount of a compound of formula (I) or (I-1) or a pharmaceutically acceptable salt thereof. The susceptible neoplasm may be selected from the group consisting of breast cancer, colon cancer, small cell lung cancer, non-small cell lung cancer, prostate cancer, endometrial cancer, gastric cancer, melanoma, pancreatic cancer, ovarian cancer, squamous cell carcinoma, carcinoma of the head and neck, esophageal carcinoma, hepatocellular carcinoma and hematologic maliginancies such as acute leukemias and aggressive lymphomas. In one particular aspect, there is provided a method of treating breast cancer in a mammal in need thereof, which comprises administering to the mammal a therapeutically effective amount of a compound of formula (I) or (I-1) or a pharmaceutically acceptable salt thereof. In one particular aspect, there is provided a method of treating ovarian cancer in a mammal in need thereof, which comprises administering to the mammal a therapeutically effective amount of a compound of formula (I) or (I-1) or a pharmaceutically acceptable salt thereof. In one particular aspect, there is provided a method of treating non-small cell lung cancer in a mammal in need thereof, which comprises administering to the mammal a therapeutically effective amount of a compound of formula (I) or (I-1) or a pharmaceutically acceptable salt thereof. In one particular aspect, there is provided a method of treating prostate cancer in a mammal in need thereof, which comprises administering to the mammal a therapeutically effective amount of a compound of formula (I) or (I-1) or a pharmaceutically acceptable salt thereof. In one particular aspect, there is provided a method of treating a hematologic malignancy in a mammal in need thereof, which comprises administering to the mammal a therapeutically effective amount of a compound of formula (I) or (I-1) or a pharmaceutically acceptable salt thereof.

In a seventh aspect of the invention, there is provided a method for treating a condition characterized by inappropriate cellular proliferation. The method comprises contacting the cell with a therapeutically effective amount of a compound of formula (I) or (I-1) or a pharmaceutically acceptable salt thereof.

In an eighth aspect, the present invention provides a method for inhibiting proliferation of a cell. The method comprises contacting the cell with an amount of a compound of formula (I) or (I-1) or a pharmaceutically acceptable salt thereof.

In another aspect, the present invention provides a method for inhibiting mitosis in a cell. The method comprises administering to the cell an amount of a compound of formula (I) or (I-1) or a pharmaceutically acceptable salt thereof.

In another aspect, the present invention provides a process for preparing a compound of formula (I) wherein Ring A is at the 5-position (i.e. a compound of formula (I-5)) or a pharmaceutically acceptable salt thereof. The process comprises either:

### Process A:

reacting the compound of formula (VII-5):

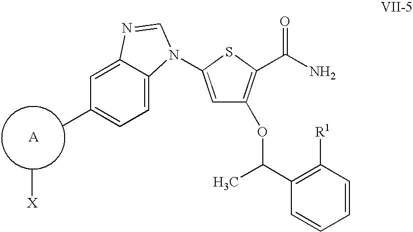

wherein Me is methyl, X is a halogen, and all other variables are as defined above;
with a compound of formula (H—Y—R²—R³) to prepare a compound of formula (I-5):

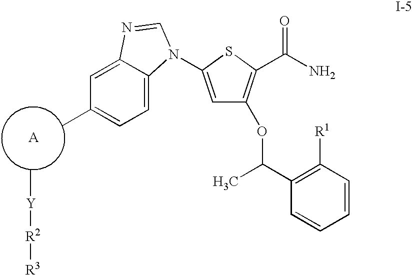

wherein all variables are as defined above; or

### Process B:

reacting a compound of formula (VI-5):

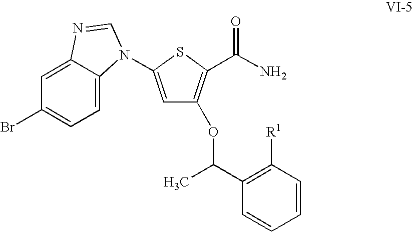

wherein all variables are as defined above
with an aryl metal reagent of formula (VIII-A):

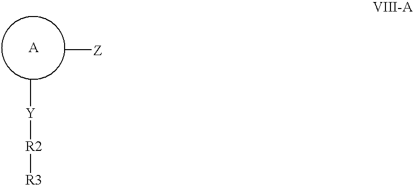

wherein Z is a a boronic acid, boronic ester, trifluoroborate salt, stannane or zinc halide, and all other variables are as defined above;
to prepare a compound of formula (I-5).

In another aspect, the present invention provides a process for preparing a compound of formula (I) or a pharmaceutically acceptable salt thereof. The process comprises either:

### Process A:

reacting a compound of formula (VII-5,6):

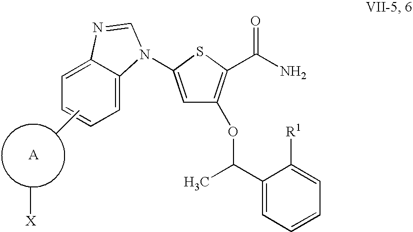

wherein X is halogen and all other variables are as defined above and Ring A is bound to the 5- or 6-position of the benzimidazole;
with a compound of formula (H—Y—R²—R³) to prepare a compound of formula (I); or

### Process B:

reacting a compound of formula (VI-5,6):

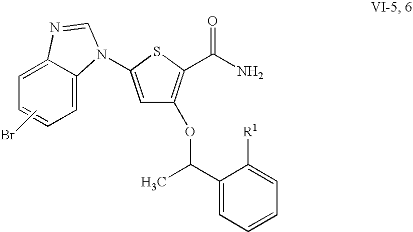

wherein all variables are as defined above and Br is bound to the 5- or 6-position of the benzimidazole;
with an aryl metal reagent of formula (VIII-A):

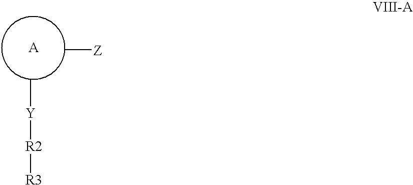

wherein Z is a a boronic acid, boronic ester, trifluoroborate salt, stannane or zinc halide, and all other variables are as defined above;
to prepare a compound of formula (I).

In another aspect, the present invention provides a compound of formula (I) or (I-1) or a pharmaceutically acceptable salt thereof for use in therapy.

In yet another aspect, the present invention provides a compound of formula (I) or (I-1) or a pharmaceutically acceptable salt thereof for use in the treatment of a condition mediated by PLK in a mammal in need thereof.

In yet another aspect, the present invention provides a compound of formula (I) or (I-1) or a pharmaceutically acceptable salt thereof for use in the treatment of a susceptible neoplasm in a mammal. In one particular aspect, there is provided a compound of formula (I) or (I-1) or a pharmaceutically acceptable salt thereof, for use in the treatment of breast cancer, ovarian cancer, non-small cell lung cancer, prostate cancer, or a hematologic malignancy in a mammal.

In another aspect, the present invention provides a compound of formula (I) or (I-1) or a pharmaceutically acceptable salt thereof, for use in the treatment of a condition characterized by inappropriate cellular proliferation.

In yet another aspect, the present invention provides a compound of formula (I) or (I-1) or a pharmaceutically acceptable salt thereof, for use in inhibiting proliferation of a cell.

In yet another aspect, the present invention provides a compound of formula (I) or (I-1) or a pharmaceutically acceptable salt thereof, for use in inhibiting mitosis in a cell.

In yet another aspect, the present invention provides the use of a compound of formula (I) or (I-1) or a pharmaceutically acceptable salt thereof, for the preparation of a medicament for the treatment of condition mediated by PLK in a mammal.

In yet another aspect, the present invention provides the use of a compound of formula (I) or (I-1) or a pharmaceutically acceptable salt thereof, for the preparation of a medicament for the treatment of a susceptible neoplasm, such as breast cancer, colon cancer, small cell lung cancer, non-small cell lung cancer, prostate cancer, endometrial cancer, gastric cancer, melanoma, pancreatic cancer, ovarian cancer, squamous cell carcinoma, carcinoma of the head and neck, esophageal carcinoma, hepatocellular carcinoma and hematologic maliginancies, in a mammal.

In yet another aspect, the present invention provides the use of a compound of formula (I) or (I-1) or a pharmaceutically acceptable salt thereof, for the preparation of a medicament for the treatment of breast cancer, ovarian cancer, non small cell lung cancer, prostate cancer or a hematologic malignancy in a mammal.

In yet another aspect, the present invention provides the use of a compound of formula (I) or (I-1) or a pharmaceutically acceptable salt thereof, for the treatment of a condition characterized by inappropriate cellular proliferation in a mammal.

In yet another aspect, the present invention provides the use a compound of formula (I) or (I-1) or a pharmaceutically acceptable salt thereof, for the preparation of a medicament for inhibiting proliferation of a cell.

In yet another aspect, the present invention provides the use of a compound of formula (I) or (I-1) or a pharmaceutically acceptable salt thereof, for the preparation of a medicament for inhibiting mitosis in a cell.

In yet another aspect, the present invention provides a pharmaceutical composition comprising a compound of formula (I) or (I-1) or a pharmaceutically acceptable salt thereof, for use in the treatment of a susceptible neoplasm, such as breast cancer, colon cancer, small cell lung cancer, non-small cell lung cancer, prostate cancer, endometrial cancer, gastric cancer, melanoma, pancreatic cancer, ovarian cancer, squamous cell carcinoma, carcinoma of the head and neck, esophageal carcinoma, hepatocellular carcinoma and hematologic maliginancies, in a mammal.

### DETAILED DESCRIPTION OF THE INVENTION

As used herein, “compound(s) of the invention” means a compound of formula (I) or a pharmaceutically acceptable salt thereof in any form, including amorphous, non-crystalline, crystalline, any particular morphic form, solvates (including specifically hydrates) and enantiomerically enriched forms such as the enantiomerically enriched compound of formula (I-1).

Similarly, with respect to isolatable intermediates such as for example, compounds of formula (III-5), (IV-5), (V-5), (VI-5,6), (VII-5,6) (XVIII), (IXX), (XX), (XXII), (XXIV), (XXV) and (XXVI) (among others described below) the phrase “a compound of formula (number)” means a compound having that formula and pharmaceutically acceptable salts thereof in any form.

As used herein, the terms “alkyl” (and “alkylene”) refer to straight or branched hydrocarbon chains containing from 1 to 8 carbon atoms. Examples of “alkyl” as used herein include, but are not limited to, methyl, ethyl, n-propyl, isopropyl, n-butyl, isobutyl, tert-butyl and n-pentyl. Examples of “alkylene” as used herein include, but are not limited to, methylene, ethylene, propylene, isopropylene, butylene, and isobutylene.

The term “halo” or “halogen” refers to fluorine, chlorine, bromine and iodine.

The term “oxo” as used herein refers to the group ═O attached directly to a carbon or heteroatom (N or S) atom of a hydrocarbon (i.e., cycloalkenyl or aryl)heterocyclic or heteroaryl ring. Thus, oxo includes —N-oxides, sulfones and sulfoxides wherein the N or S are atoms of a heterocyclic or heteroaryl ring.

The terms “heterocycle” and “heterocyclic” refer to monocyclic saturated or unsaturated non-aromatic cyclic groups having the specified number of members and containing 1, 2 or 3 heteroatoms selected from N, O and S (unless a different number of heteroatoms is specified). Examples of particular heterocyclic groups include but are not limited to tetrahydrofuran, dihydropyran, tetrahydropyran, pyran, thietane, 1,4-dioxane, 1,3-dioxane, 1,3-dioxalane, piperidine, piperazine, tetrahydropyrimidine, pyrrolidine, morpholine, thiomorpholine, thiazolidine, oxazolidine, tetrahydrothiopyran, tetrahydrothiophene, and the like.

The term “heteroaryl” refers to aromatic monocyclic groups, having the specified number of members and containing 1, 2, or 3 heteroatoms selected from N, O and S (unless a different number of heteroatoms is specified). Examples of particular heteroaryl groups include but are not limited to furan, thiophene, pyrrole, imidazole, pyrazole, triazole, tetrazole, thiazole, oxazole, isoxazole, oxadiazole, thiadiazole, isothiazole, pyridine, pyridazine, pyrazine and pyrimidine.

The term “members” (and variants thereof e.g., “membered”) in the context of heterocyclic and heteroaryl groups refers to the total atoms, carbon and heteroatoms N, O and/or S, which form the ring. Thus, an example of a 6-membered heterocyclic ring is piperidine and an example of a 6-membered heteroaryl ring is pyridine.

As used herein, the term “optionally” means that the subsequently described event(s) may or may not occur, and includes both event(s) that occur and events that do not occur.

The present invention provides compounds of formula (I):

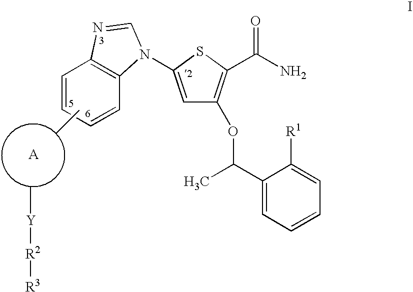

wherein
R¹ is F, Cl, CH₃ or CF₃;
Ring A is selected from phenyl and 5-6 membered heteroaryl having 1 or 2 heteroatoms selected from N, O and S;
Y is selected from —O—, —N(R⁴)—, —N(R⁴)—C(O)— and —N(R⁴)—R²—OC(O)—;
R² is linear or branched C₁₋₄alkylene optionally substituted once by —OH;
R³ is selected from —OR⁴, —N(R⁴)₂ and Het;
each R⁴ is the same or different and is independently H or C₁₋₄alkyl;
Het is a 5-6 member heterocycle having 1 or 2 heteroatoms selected from N, O and S and optionally substituted by 1 or 2 substituents selected from halo, C₁₋₄alkyl, oxo, —OR⁴, —C(O)R⁴, —C(O)₂R⁴, —SO₂R⁴— and CN;
   and pharmaceutically acceptable salts thereof.

The substituted Ring A moiety:

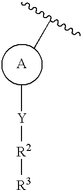

may be bound to either the 5- or 6-position of the benzimidazole as numbered in formula (I).

Thus, the present invention includes compounds of formula (I-5) and (I-6):

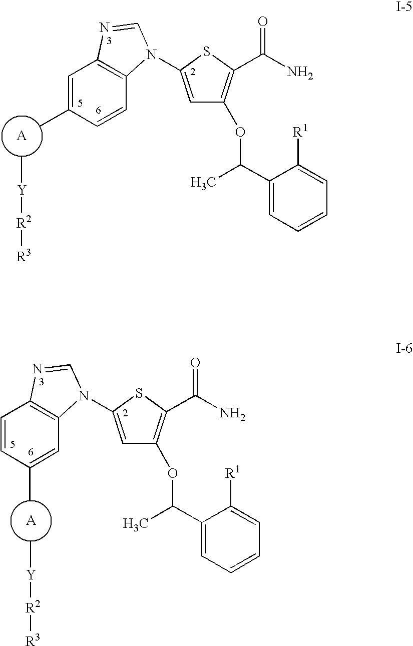

wherein all variables are as defined above.

In one embodiment, the compounds of formula (I) are defined wherein Ring A is phenyl or a 6-membered heteroaryl ring having 1 or 2 heteroatoms selected from N, O and S. In a particular embodiment, the compounds of formula (I) are defined wherein Ring A is phenyl or a 6-membered heteroaryl ring having 1 or 2 N. In one preferred embodiment, Ring A is phenyl, pyridine or pyrimidine. In one embodiment, Ring A is pyridine. In another particular embodiment, Ring A is pyrimidine.

Specific examples of the substituted Ring A moiety include but are not limited to:

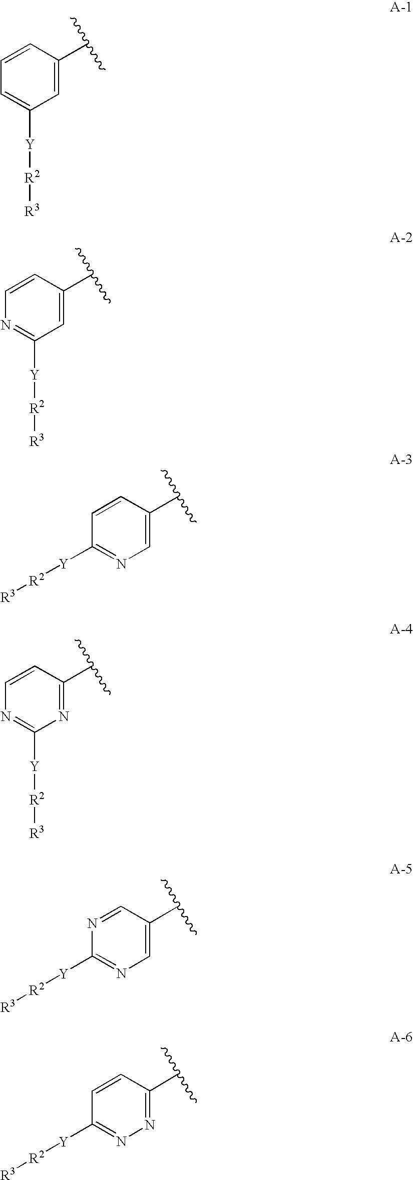

wherein all variables are as defined above.

In one embodiment, the compounds of formula (I) are defined wherein Y is selected from —N(R⁴)— and —N(R⁴)—C(O)—. In one particular embodiment, Y is —N(H)—, —N(CH₃)— or —N(H)—C(O)—. In another particular embodiment, Y is —N(H)— or —N(CH₃)—. In another preferred embodiment, Y is —N(H)—.

In one embodiment, the compounds of formula (I) are defined wherein R² is linear or branched C₁₋₃alkylene optionally substituted once by —OH. In one particular embodiment, wherein Y is —N(R⁴)—C(O)—, R² is methylene. In one preferred embodiment, wherein Y is —N(R⁴)—, R² is ethylene, propylene or isopropylene, each optionally substituted once by —OH.

In one embodiment, the compounds of formula (I) are defined wherein R³ is —N(R⁴)₂ or Het. In one preferred embodiment, R³ is —NH₂, —N(H)(CH₃), or —N(CH₃)₂. In another particular embodiment, R³ is Het optionally substituted 1 or 2 times by a substituent selected from halo, C₁₋₄alkyl, oxo, —OR⁴, —C(O)R⁴, —C(O)₂R⁴, —SO₂R⁴ and —CN; including N-oxides, N-methyl, N-ethyl, etc. In one preferred embodiment, wherein R³ is Het, Het is pyrrolidine, piperizine, or morpholine, each optionally substituted 1 or 2 times by C₁₋₄alkyl, oxo, —OR⁴, —C(O)R⁴, —SO₂R⁴— or CN; including N-oxides, N-methyl, N-ethyl, etc. In preferred embodiment wherein R³ is Het, Het is pyrrolidine, piperizine, or morpholine, each optionally substituted 1 or 2 times by C₁₋₄alkyl, including N-methyl, N-ethyl, etc.

Specific examples of the Y—R²—R³ moeity include but are not limited to:

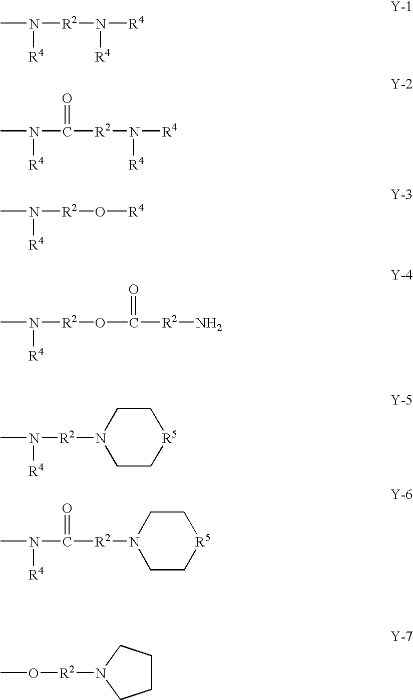

wherein R⁵ is selected from —O—, —N(H)— and —N(CH₃)—, and all other variables are as defined above.

Compounds of the invention exist in stereoisomeric forms (e.g. they contain one or more chiral or asymmetric carbon atoms). The term “chiral” refers to a molecule that is not superimposable on its mirror image. The term “achiral” refers to a molecule that is superimposable on its mirror image.

The term “stereoisomers” refers to compounds which are have a common chemical constitution but differ in the arrangement of the atoms or groups in space. Stereoisomers may be optical isomers or geometric isomers. Optical isomers include both enantiomers and diastereomers. An “enantiomer” is one of a pair of optical isomers containing a chiral carbon atom whose molecular configuration has left- and right-hand (chiral) forms. That is, “enantiomer” refers to each of a pair of optical isomers of a compound which are non-superimposable mirror images of one another. A “diastereomer” is one of a pair of optical isomers of a compound with two or more centers of dissymmetry and whose molecules are not mirror images of one another. The nomenclature of a chiral center is governed by the (R)—(S) system. Whether a particular compound is designated as the “R” or “S” enantiomer according to the system depends upon the nature of the atoms or groups which are bound to the chiral carbon.

Enantiomers differ in their behavior toward plane-polarized light, that is, their optical activity. An enantiomer that rotates plane-polarized light in a clockwise direction is said to be dextrorotatory and is designated by the symbol “d” or “(+)” for positive rotation. An enantiomer that rotates plane-polarized light in the counterclockwise direction is said to be levorotatory and is designated by the symbol “I” or “(−)” for negative rotation. There is no correlation between the configuration of enantiomers and the direction in which they rotate plane-polarized light. There is also no necessary correlation between the (R) and (S) designation and the direction of rotation of the plane-polarized light. The optical activity or direction of rotation of plane-polarized light, of an enantiomer of a compound of the invention may be determined using conventional techniques.

The compounds of the present invention may be racemic, enantiomerically enriched or enantiomerically pure form. The terms “racemate” and “racemic mixture” as used herein refer to a mixture of optical isomers (e.g., enantiomers or diastereomers) of a compound in equal, i.e. 50:50 proportion.

The term “enantiomerically enriched” as used herein refers to preparations comprising a mixture of optical isomers in which the quantity of one enantiomer is higher than the quantity of the other. Thus, “enantiomerically enriched” refers to mixtures of optical isomers wherein the ratio of enantiomer is greater than 50:50. An enantiomerically enriched compound comprises greater than 50% by weight of one enantiomer relative to the other. For example enantiomerically enriched 5-[5-(2-{[2-(Dimethylamino)ethyl]amino}-4-pyridinyl)-1H-benzimidazol-1-yl]-3-({(1R)-1-[2-(trifluoromethyl)phenyl]ethyl}-oxy)-2-thiophenecarboxamide refers to a composition comprising greater than 50% by weight of the (R)-enantiomer relative to the (S)-enantiomer of the compound. In one embodiment, an enantiomerically enriched compound comprises at least 75% by weight of one enantiomer relative to the other. In another embodiment, an enantiomerically enriched compound comprises at least 80% by weight of one enantiomer relative to the other. In one particular embodiment, an enantiomerically enriched compound comprises at least 85% by weight of one enantiomer relative to the other. The present invention includes enantiomerically enriched forms of each compound of formula (I).

The term “enantiomerically pure” as used herein refers to enantiomerically enriched compounds comprising at least 90% by weight of one enantiomer relative to the other. In one embodiment, an enantiomerically pure compound comprises at least 95% by weight of one enantiomer relative to the other. In one particular embodiment, an enantiomerically pure compound comprises at least 99% by weight of one enantiomer relative to the other. The present invention includes enantiomerically enriched forms of each compound of formula (I).

In one embodiment, the present invention provides an enantiomerically enriched compound of formula (I), having the stereochemistry depicted in formula (I-1):

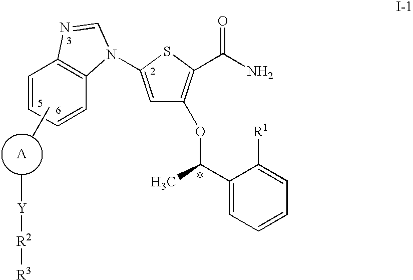

wherein * indicates the chiral carbon and all variables are as defined above.

The foregoing specific embodiments of the invention described above for the variables defining compounds of formula (I) are equally applicable to compounds of formula (I-1). Further, it is to be understood that the present invention includes all combinations and subsets of the particular groups defined hereinabove.

Specific examples of compounds within the scope of the present invention include those recited in the Examples which follow and pharmaceutically acceptable salts thereof.

It will be appreciated by those skilled in the art that the compounds of the present invention may be utilized in the form of a pharmaceutically acceptable salt thereof. The pharmaceutically acceptable salts of the compounds of the present invention (or the enantiomerically enriched or pure forms thereof) include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, trifluoroacetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic (mesylate), naphthalene-2-sulfonic, benzenesulfonic hydroxynaphthoic, hydroiodic, malic, steroic, tannic and the like.

Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

Processes for preparing pharmaceutically acceptable salts of the compounds of the invention are conventional in the art. See, e.g., Burger's Medicinal Chemistry And Drug Discovery 5th Edition, Vol 1: Principles And Practice.

As will be apparent to those skilled in the art, in the processes described below for the preparation of the compounds of the invention, certain intermediates, may alternatively be in the form of pharmaceutically acceptable salts of the compound. Those terms as applied to any intermediate employed in the process of preparing the compounds of the invention have the same meanings as noted above with respect to the compounds of the invention. Processes for preparing pharmaceutically acceptable salts of such intermediates are known in the art and are analogous to the process for preparing pharmaceutically acceptable salts of the compounds of the invention.

The compounds of the present invention are typically inhibitors of PLK, in particular, PLK1. By PLK inhibitor is meant a compound which exhibits pIC₅₀ greater than 6 in the PLK Inhibition assay described below in the examples or an IC₅₀ less than 10 μM in the Cell-Titer Glo or Methylene Blue Cell Growth Inhibition assays described below in the examples; in a more particular embodiment, the PLK inhibitor exhibits a pIC₅₀ greater than 7 in the PLK Inhibition assay or an IC₅₀ less than 1 μM in the Cell-Titer Glo or Methylene Blue Cell Growth Inhibition assay using the methods described in the examples below.

The present invention further provides compounds of the invention for use in medical therapy in an animal, e.g. a mammal such as a human. In particular, the present invention provides compounds for use in the treatment of a condition mediated by PLK, particularly PLK1. More specifically, the present invention provides compounds for use in the treatment of a condition mediated by PLK which is susceptible to treatment with a PLK, particularly PLK1, inhibitor. The present invention also provides compounds for use in the treatment of a susceptible neoplasm (defined hereinbelow). In particular, the present invention provides compounds for use in the treatment of a variety of solid tumors including but not limited to breast cancer, ovarian cancer, non-small cell lung cancer and prostate cancer as well as hematologic malignancies including but not limited to acute leukemias and aggressive lymphomas. “Acute leukemias” includes both acute myeloid leukemias and acute lymphoid leukemias. See, N. Harris, et al., J. Clin. One. (1999) 17(12):3835-3849. “Aggressive lymphomas” is a term of art. See, J. Chan, Hematological One. (2001) 19:129-150.

The present invention provides compounds for use in treating a condition characterized by inappropriate cellular proliferation. The present invention also provides compounds for use in inhibiting proliferation of a cell. The present invention also provides compounds for use in inhibiting mitosis in a cell.

The present invention provides methods for the treatment of several conditions or diseases, all of which comprise the step of administering a therapeutically effective amount of a compound of the invention. As used herein, the term “treatment” refers to alleviating the specified condition, eliminating or reducing the symptoms of the condition, slowing or eliminating the progression of the condition and preventing or delaying the reoccurrance of the condition in a previously afflicted subject.

As used herein, the term “therapeutically effective amount” means an amount of a compound of the invention which is sufficient, in the subject to which it is administered, to elicit the biological or medical response of a cell culture, tissue, system, animal (including human) that is being sought, for instance, by a researcher or clinician. For example, a therapeutically effective amount of a compound of the invention for the treatment of a condition mediated by PLK, particularly PLK1, is an amount sufficient to treat the PLK mediated condition in the subject. Similarly, a therapeutically effective amount of a compound of the invention for the treatment of a susceptible neoplasm is an amount sufficient to treat the susceptible neoplasm in the subject. In one embodiment of the present invention, the therapeutically effective amount of a compound of the invention is an amount sufficient to treat breast cancer in a human in need thereof. In one embodiment of the present invention, a therapeutically effective amount of a compound of the invention is an amount sufficient to regulate, modulate, bind or inhibit PLK, particularly PLK1.

The precise therapeutically effective amount of the compounds of the invention will depend on a number of factors including, but not limited to, the age and weight of the subject being treated, the precise condition or disease requiring treatment and its severity, the nature of the formulation, and the route of administration, and will ultimately be at the discretion of the attendant physician or veternarian. Typically, the compound of the invention will be given for treatment in the range of 0.1 to 200 mg/kg body weight of recipient (animal) per day, per dose or per cycle of treatment and more usually in the range of 1 to 100 mg/kg body weight per day, per dose or per cycle of treatment. Acceptable daily dosages, may be from about 0.1 to about 2000 mg per day, per dose or per cycle of treatment, and preferably from about 0.1 to about 500 mg per day, per dose or per cycle of treatment.

As one aspect, the present invention provides methods of regulating, modulating, binding, or inhibiting PLK for the treatment of conditions mediated by PLK, particularly PLK1. “Regulating, modulating, binding or inhibiting PLK” refers to regulating, modulating, binding or inhibiting PLK, particularly PLK1 activity, as well as regulating, modulating, binding or inhibiting overexpression of PLK, particularly PLK1. Such conditions include certain neoplasms (including cancers and tumors) which have been associated with PLK, particularly PLK1, and conditions characterized by inappropriate cellular proliferation.

The present invention provides a method for treating a condition mediated by PLK, particularly PLK1, which condition is susceptible to treatment with a PLK, particularly PLK1, inhibitor. The method comprises administering to the animal a therapeutically effective amount of the compound of the invention. This method and other methods of the present invention are useful for the treatment of an animal such as a mammal and in particular humans. Conditions which are mediated by PLK which are susceptible to treatment with a PLK, particularly PLK1, inhibitor, are known in the art and include but are not limited to neoplasms and conditions characterized by inappropriate cellular proliferation.

The present invention also provides a method for treating a susceptible neoplasm (cancer or tumor) in an animal such as a mammal (e.g., a human) in need thereof, which method comprises administering to the animal a therapeutically effective amount of the compound of the invention. “Susceptible neoplasm” as used herein refers to neoplasms which are susceptible to treatment with a PLK, particularly PLK1, inhibitor. Neoplasms which have been associated with PLK and are therefore susceptible to treatment with a PLK inhibitor are known in the art, and include both primary and metastatic tumors and cancers. See e.g., M. Whitfield et al., (2006) Nature Reviews/Cancer 6:99. For example, susceptible neoplasms within the scope of the present invention include but are not limited to breast cancer, colon cancer, lung cancer (including small cell lung cancer and non-small cell lung cancer), prostate cancer, endometrial cancer, gastric cancer, melanoma, ovarian cancer, pancreatic cancer, squamous cell carcinoma, carcinoma of the head and neck, esophageal carcinoma, hepatocellular carcinoma and hematologic malignancies such as acute leukemias and aggressive lymphomas. In one particular embodiment, the present invention provides a method of treating breast cancer in an animal, such as a mammal (e.g., a human) in need thereof by administering a therapeutically effective amount of a compound of the present invention. In another particular embodiment, the present invention provides a method of treating ovarian cancer in an animal, such as a mammal (e.g., a human) in need thereof by administering a therapeutically effective amount of a compound of the present invention. In another particular embodiment, the present invention provides a method of treating non-small cell lung cancer in an animal, such as a mammal (e.g., a human) in need thereof by administering a therapeutically effective amount of a compound of the present invention. In another particular embodiment, the present invention provides a method of treating prostate cancer in an animal, such as a mammal (e.g., a human) in need thereof by administering a therapeutically effective amount of a compound of the present invention. In another particular embodiment, the present invention provides a method of treating hematologic malignancies including acute leukemias and aggressive lymphomas in an animal, such as a mammal (e.g., a human) in need thereof by administering a therapeutically effective amount of a compound of the present invention.

The compounds of the invention can be used alone in the treatment of such susceptible neoplasms or can be used to provide additive or synergistic effects with one or more other compounds of the invention, or in combination with certain existing chemotherapies and/or other anti-neoplastic therapies. In addition, the compounds of the invention can be used to restore effectiveness of certain existing chemotherapies and/or other anti-neoplastic therapies. As used herein, “anti-neoplastic therapies” includes but is not limited to cytotoxic chemotherapy, hormonal therapy, targeted kinase inhibitors, therapeutic monoclonal antibodies, surgery and radiation therapy.

The present invention also provides a method for treating a condition characterized by inappropriate cellular proliferation in an animal, such as a mammal (e.g., a human) in need thereof. The method comprises administering a therapeutically effective amount of a compound of the present invention. By “inappropriate cellular proliferation” is meant cellular proliferation resulting from inappropriate cell growth, cellular proliferation resulting from excessive cell division, cellular proliferation resulting from cell division at an accelerated rate, cellular proliferation resulting from inappropriate cell survival, and/or cellular proliferation in a normal cell occurring at a normal rate, which is nevertheless undesired. Conditions characterized by inappropriate cellular proliferation include but are not limited to neoplasms, blood vessel proliferative disorders, fibrotic disorders, mesangial cell proliferative disorders and inflammatory/immune-mediated diseases. Blood vessel proliferative disorders include arthritis and restenosis. Fibrotic disorders include hepatic cirrhosis and atherosclerosis. Mesangial cell proliferative disorders include glomerulonephritis, malignant nephrosclerosis and glomerulopathies. Inflammatory/immune-mediated disorders include psoriasis, chronic wound healing, organ transplant rejection, thrombotic microangiopathy syndromes, and neurodegenerative diseases. Osteoarthritis and other osteoclast proliferation dependent diseases of excess bone resorbtion are examples of conditions characterized by inappropriate cellular proliferation in which the cellular proliferation occurs in normal cells at a normal rate, but is nevertheless undesired.

The present invention also provides a method for inhibiting proliferation of a cell, which method comprises contacting the cell with an amount of a compound of the invention sufficient to inhibit proliferation of the cell. In one particular embodiment, the cell is a neoplastic cell. In one particular embodiment, the cell is an inappropriately proliferative cell. The term “inappropriately proliferative cell” as used herein refers to cells that grow inappropriately (abnormally), cells that divide excessively or at an accelerated rate, cells that inappropriately (abnormally) survive and/or normal cells that proliferate at a normal rate but for which proliferation is undesired. Neoplastic cells (including cancer cells) are an example of inappropriately proliferative cells but are not the only inappropriately proliferative cells.

PLK is essential for cellular mitosis and accordingly, the compounds of the invention are believed to be effective for inhibiting mitosis. “Inhibiting mitosis” refers to inhibiting the entry into the M phase of the cell cycle, inhibiting the normal progression of the M phase of the cell cycle once M phase has been entered and inhibiting the normal exit from the M phase of the cell cycle. Thus, the compounds of the present invention may inhibit mitosis by inhibiting the cell's entry into mitosis, by inhibiting the cell's progression through mitosis or by inhibiting the cell's exit from mitosis. As one aspect, the present invention provides a method for inhibiting mitosis in a cell, which method comprises administering to the cell an amount of a compound of the invention sufficient to inhibit mitosis. In one particular embodiment, the cell is a neoplastic cell. In one particular embodiment, the cell is an inappropriately proliferative cell.

The present invention also provides the use of a compound of the invention for the preparation of a medicament for the treatment of condition mediated by PLK, particularly PLK1, in an animal, such as a mammal (e.g., a human), which condition is susceptible to treatment with a PLK, particularly PLK1, inhibitor. The present invention further provides the use of a compound for the preparation of a medicament for the treatment of a susceptible neoplasm in an animal, particularly a mammal (e.g., a human). In particular, the present invention provides the use of a compound for the preparation of a medicament for the treatment of a breast cancer. The present invention also provides the use of a compound for the preparation of a medicament for the treatment of ovarian cancer. The present invention provides the use of a compound for the preparation of a medicament for the treatment of non-small cell lung cancer. The present invention provides the use of a compound for the preparation of a medicament for the treatment of prostate cancer. The present invention provides the use of a compound for the preparation of a medicament for the treatment of hematologic malignancies such as acute leukemias and aggressive lymphomas. The present invention further provides the use of a compound for the preparation of a medicament for the treatment of a condition characterized by inappropriate cellular proliferation. The present invention further provides the use of a compound for the preparation of a medicament for inhibiting proliferation of a cell. The present invention further provides the use of a compound for the preparation of a medicament for inhibiting mitosis in a cell.

While it is possible that, for use in therapy, a therapeutically effective amount of a compound of the invention may be administered as the raw chemical, it is typically presented as the active ingredient of a pharmaceutical composition or formulation. Accordingly, the invention further provides a pharmaceutical composition comprising a compound of the invention. The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the invention with one or more pharmaceutically acceptable carriers, diluents and/or excipients.

Pharmaceutical formulations may be presented in unit dose form containing a predetermined amount of active ingredient per unit dose. Such a unit may contain a therapeutically effective dose of the compound of the invention or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quarternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of active ingredient. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be delivered by the use of monoclonal antibodies as individual carriers to which the compound molecules are coupled. The compounds may also be coupled with soluble polymers as targetable drug carriers. Such polymers can include peptides, polyvinylpyrrolidone, pyran copolymer, polyhydroxypropylmethacrylamide—phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986). Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream.

When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators. Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

In the above-described methods of treatment and uses, a compound of the invention may be employed alone, in combination with one or more other compounds of the invention or in combination with other therapeutic agents and/or in combination with other anti-neoplastic therapies. In particular, in methods of treating conditions mediated by PLK and methods of treating susceptible neoplasms, combination with other chemotherapeutic agents is envisaged as well as combination with surgical therapy and radiation therapy. The term “chemotherapeutic” as used herein refers to any chemical agent having a therapeutic effect on the subject to which it is administered. “Chemotherapeutic” agents include but are not limited to anti-neoplastic agents, analgesics and anti-emetics. As used herein, “anti-neoplastic agents” include both cytostatic and cytotoxic agents such as but not limited to cytotoxic chemotherapy, hormonal therapy, targeted kinase inhibitors and therapeutic monoclonal antibodies. Combination therapies according to the present invention thus comprise the administration of at least one compound of the invention and the use of at least one other cancer treatment method. In one embodiment, combination therapies according to the present invention comprise the administration of at least one compound of the invention and at least one other chemotherapeutic agent. In one particular embodiment, the present invention comprises the administration of at least one compound of the invention and at least one anti-neoplastic agent. As an additional aspect, the present invention provides the methods of treatment and uses as described above, which comprise administering a compound of the invention together with at least one chemotherapeutic agent. In one particular embodiment, the chemotherapeutic agent is an anti-neoplastic agent. In another embodiment, the present invention provides a pharmaceutical composition as described above further comprising at least one other chemotherapeutic agent, more particularly, the chemotherapeutic agent is an anti-neoplastic agent.

Typically, any chemotherapeutic agent that has activity versus a susceptible neoplasm being treated may be utilized in combination with the compounds of the invention, provided that the particular agent is clinically compatible with therapy employing a compound of the invention. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphor-ines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids. Examples of diterpenoids include, but are not limited to, paclitaxel and its analog docetaxel. Examples of vinca alkaloids include, but are not limited to, vinblastine, vincristine, and vinorelbine.

Platinum coordination complexes are non-phase specific anti-neoplastic agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, oxaliplatin, cisplatin and carboplatin.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Antibiotic chemotherapeutic agents are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthracyclins such as daunorubicin and doxorubicin; and bleomycins.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine and thioguanine.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin. Hormones and hormonal analogues are useful compounds for treating cancers in which there is a relationship between the hormone(s) and growth and/or lack of growth of the cancer. Examples of hormones and hormonal analogues believed to be useful in the treatment of neoplasms include, but are not limited to, adrenocorti-costeroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene and iodoxyfene useful in the treatment of hormone dependent breast carcinoma; and gonadotropin-releasing hormone (GnRH) and analogues thereof, such as goserelin acetate and leuprolide, which stimulate the release of leutinizing hormone (LH) and/or follicle stimulating hormone (FSH) with short-term or intermittent use but lead to suppression of LH and FSH with long-term use indicated for the treatment prostatic carcinoma, and hormone dependent breast carcinoma.

Signal transduction pathway inhibitors are those inhibitors which block or inhibit a chemical process which evokes an intracellular change. As used herein this change is cell proliferation, survival, angiogenesis or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are sometimes termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr, ErbB2 and ErbB4), platelet derived growth factor receptor (PDGFr), vascular endothelial growth factor receptor (VEGFR), tyrosine kinase with immunoglobulin-like and epidermal growth factor homology domains (TIE-2), insulin growth factor-I receptor (IGF-I), macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth factor receptors are under development and include ligand antagonists, antibodies, tyrosine kinase inhibitors, anti-sense oligonucleotides and aptamers. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth Factor Receptors as Targets”, New Molecular Targets for Cancer Chemotherapy, Ed. Workman, Paul and Kerr, David, CRC Press 1994, London.

Tyrosine kinases, which are not growth factor receptor kinases are termed non-receptor tyrosine kinases. Non-receptor tyrosine kinases useful in the present invention, which are targets or potential targets of anti-neoplastic drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual Review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (Rafk), Mitogen or Extracellular Regulated Kinase (MEKs), and Extracellular Regulated Kinases (ERKs); and Protein kinase C family member blockers including blockers of subtypes of PKCs (alpha, beta, gamma, epsilon, mu, lambda, iota, zeta), IkB kinase family (IKKa, IKKb), PKB family kinases, Akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60.1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; and Martinez-lacaci, L., et al, Int. J. Cancer (2000), 88(1), 44-52.

Inhibitors of Phosphotidyl Inositol-3 Kinase family members including blockers of PI3-kinase, ATM, DNA-PK, and Ku are also useful in combination with the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer Res, (2000) 60(6), 1541-1545.

Also useful in combination with the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC Press 1994, London.

Another group of signal transduction pathway inhibitors useful in combination with the present invention are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block Ras activation in cells containing wild type mutant Ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9(2)99-102; and BioChim. Biophys. Acta, (1989) 1423(3):19-30.

As mentioned above, antibodies to receptor kinase ligand binding may also serve as signal transduction inhibitors. This group of signal transduction pathway inhibitors includes the use of humanized antibodies to the extracellular ligand binding domain of receptor tyrosine kinases. For example, Imclone C225 EGFR specific antibody (see Green, M. C. et al, Monoclonal Antibody Therapy for Solid Tumors, Cancer Treat. Rev., (2000), 26(4), 269-286); Herceptin® ErbB2 antibody (see Tyrosine Kinase Signaling in Breast Cancer:ErbB Family Receptor Tyrosine Kinases, Breast Cancer Res., 2000, 2(3), 176-183); and 2CB VEGFR2 specific antibody (see Brekken, R. A. et al, Selective Inhibition of VEGFR2Activity by a Monoclonal Anti-VEGF Antibody Blocks Tumor Growth in Mice, Cancer Res. (2000) 60, 5117-5124).

Receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Other inhibitors may be used in combination with the compounds of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with PLK inhibitors.

Agents used in immunotherapeutic regimens may also be useful in combination with the compounds of the invention.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide.

Such proapoptotic strategies using the antisense oligonucleotide strategy for bcl-2 are discussed in Water J S et al., J. Clin. Oncol. 18:1812-1823 (2000); and Kitada S et al., Antisense Res. Dev. 4:71-79 (1994).

Cell cycle signaling inhibitors inhibit molecules involved in the control of the cell cycle. Cyclin dependent kinases (CDKs) and their interaction cyclins control progression through the eukaryotic cell cycle. The coordinated activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, Rosania, et al., Exp. Opin. Ther. Patents 10(2):215-230 (2000).

In one embodiment, the methods of the present invention comprise administering to the animal a compound of the invention in combination with a signal transduction pathway inhibitor, particularly gefitinib (IRESSA®).

The methods and uses employing these combinations may comprise the administration of the compound of the invention and the other chemotherapeutic/anti-neoplastic agent either sequentially in any order or simultaneously in separate or combined pharmaceutical compositions. When combined in the same formulation it will be appreciated that the two compounds must be stable and compatible with each other and the other components of the formulation and may be formulated for administration. When formulated separately they may be provided in any convenient formulation, in such a manner as are known for such compounds in the art.

When a compound of the invention is used in combination with a chemotherapeutic agent, the dose of each compound may differ from that when the compound is used alone. Appropriate doses will be readily appreciated by those skilled in the art. The appropriate dose of the compound(s) of the invention and the other therapeutically active agent(s) and the relative timings of administration will be selected in order to achieve the desired combined therapeutic effect, and are within the expertise and discretion of the attendent clinician.

Compounds of the invention may be prepared by several synthetic routes, as depicted in the reaction schemes which follow. In particular, compounds of formula (I) wherein Ring A is at the 5-position may be prepared as follows.

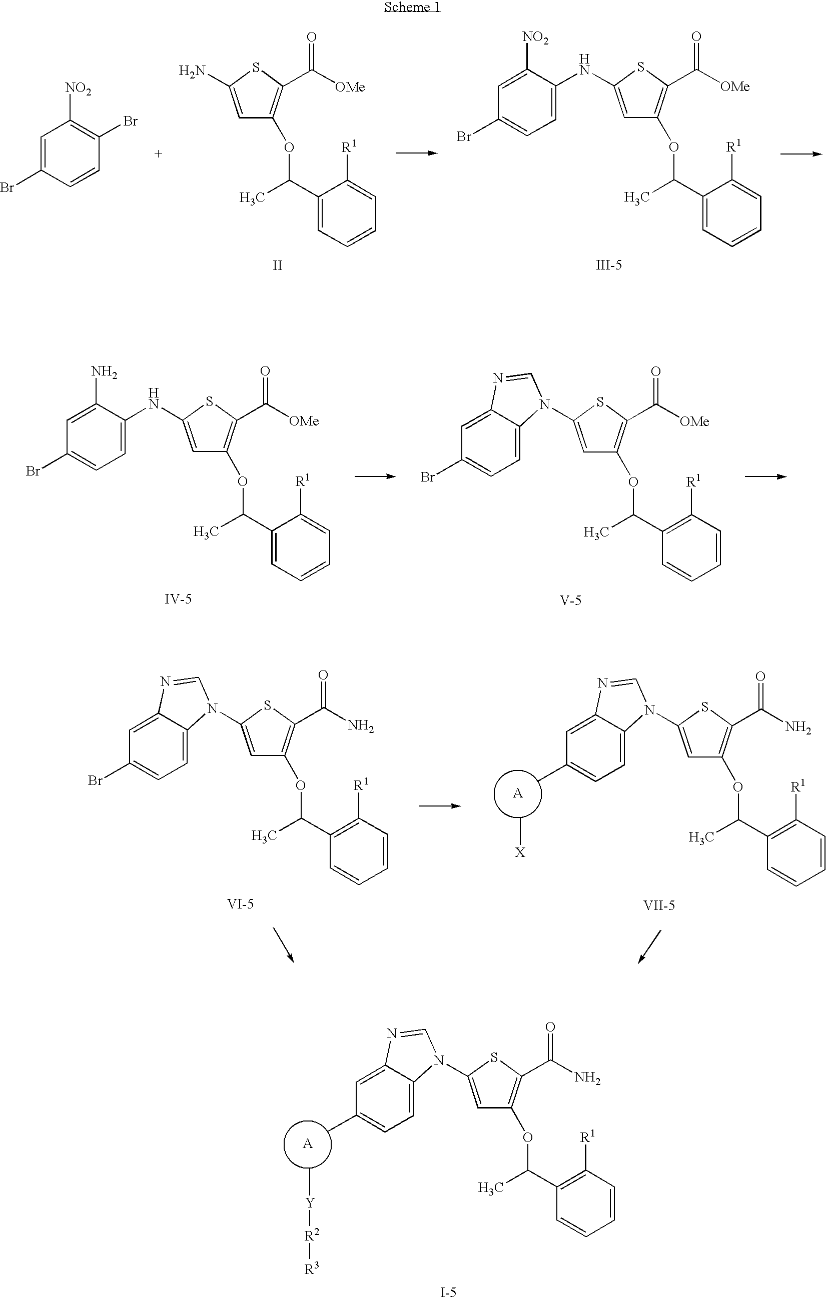

wherein:
Me is methyl; X is a halogen (preferably Cl or F); and all other variables are as defined above.

As will be apparent to those skilled in the art and as shown in the foregoing scheme, the order of the steps in the foregoing reaction is not critical to the practice of the process of the present invention. For example, the step of reacting with ammonia to convert the ester to an amide may be carried out later in the route, such as after the coupling reaction, if desired. Thus, the foregoing reaction steps may be carried out in any suitable order based upon the knowledge of those skilled in the art. Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art.

A compound of formula (I), prepared by any suitable process, may be converted into a pharmaceutically acceptable salt thereof or may be converted to a different compound of formula (I) or a pharmaceutically acceptable salt thereof using techniques described herein below and those conventional in the art.

According to one particular process of the invention, compounds of formula (I) (all formulas and all variables having been defined above) are prepared by a process which comprises the steps of:
a) reacting 2,5-dibromonitrobenzene with a compound of formula (II) to prepare a compound of formula (III-5);
b) reducing the compound of formula (III-5) to prepare a compound of formula (IV-5);
c) cyclizing the compound of formula (IV-5) to prepare a compound of formula (V-5);
d) reacting the compound of formula (V-5) with ammonia to prepare a compound of formula (VI-5);
e) reacting the compound of formula (VI-5) with a reagent of formula (VIII):

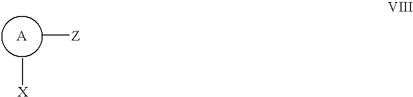

wherein X is halogen, and Z is a boronic acid, boronic ester, trifluoroborate salt (e.g., B(OH)₂ or pinacolboronate), stannane or zinc halide (e.g., zinc chloride), in the presence of a palladium (0) catalyst to prepare a compound of formula (VII-5)
f) reacting the compound of formula (VII-5) with a compound of formula (H—Y—R²—R³) to prepare a compound of formula (I-5);
g) optionally removing protecting groups;
h) optionally converting the compound of formula (I-5) to a pharmaceutically acceptable salt thereof; and
i) optionally converting the compound of formula (I-5) or a pharmaceutically acceptable salt thereof to a different compound of formula (I-5) or a pharmaceutically acceptable salt thereof.

More specifically, a compound of formula (I-5) may be prepared by nucleophilic displacement of a compound of formula (VII-5) with a compound of formula (H—Y—R²—R³) to prepare a compound of the formula (I-5). The nucleophilic displacement may be carried out in an inert solvent and heated to a temperature of from about 80° C. to about 190° C. optionally using microwave irradiation. Typically, the reaction is carried out by reacting an equimolar amount of a compound of formula (VI-5) with an equimolar amount of the compound selected from compounds of formula (H—Y—R²—R³). Suitable solvents for this reaction include but are not limited to N,N-dimethylformamide, tetrahydrofuran, dioxane, toluene, benzene, 1,2-dimethoxyethane, and 1-methyl-2-pyrrolidinone, ethanol and isopropanol. Compounds of formula H—Y—R²—R³ are commercially available or may be prepared according to the literature.

The compound of formula (VII-5) may be prepared by multiple routes. In one embodiment, a compound of formula (VII-5) may be prepared by reacting the compound of formula (VI-5) with a reagent of formula (VIII):

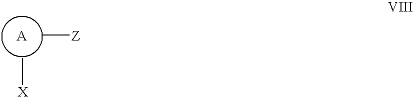

wherein X is halogen, and Z is a boronic acid, boronic ester, trifluoroborate salt (e.g., B(OH)₂ or pinacolboronate) stannane or zinc halide (e.g., zinc chloride), and Ring A is as defined above,
   using palladium-catalyzed Suzuki, Stille, or Negishi palladium-catalyzed cross-coupling techniques conventional in the art of organic synthesis.

For a review of the Suzuki cross-coupling reaction, see: Miyaura, N.; Suzuki, A. Chemical Reviews 1995, 95, 2457-2483. In the embodiment of the Suzuki cross-coupling reaction, Z is a boronic acid, boronic ester, or trifluoroborate salt. The Suzuki coupling may be carried out using a suitable catalyst such as dichloro[1,1′-bis(diphenylphosphino)ferrocene]palladium(II) dichloromethane adduct, a base such as aqueous sodium carbonate or triethylamine, and a suitable inert solvent such as N,N-dimethylacetamide or n-propanol, optionally in the presence of microwave irradiation, at temperatures from about 50° C. to about 150° C.

For a review of the Stille cross-coupling reaction, see: Mitchell, T. N. Synthesis 1992, 803-815. In the embodiment of the Stille cross-coupling reaction, Z is typically a stannane. The Stille coupling may be carried out using tetrakis(triphenylphoshine)palladium (0) as the catalyst, in the presence of promoters such as cesium fluoride and copper (I) iodide, in a suitable inert solvent such as N,N-dimethylformamide at a temperature of about 45° C.

For a review of the Negishi cross-coupling reaction, see: Negishi, E.; Zingzhong, T. Z.; Qian, M.; Hu, Q.; Huang, Z. Metal Catalyzed Cross-Coupling Reactions (2^{nd} Edition), 2004, 2, 815-889. In the embodiment of the Negishi cross-coupling reaction, Z is a zinc halide. The Negishi coupling may be carried out using dichloro[1,1′-bis(diphenylphosphino)-ferrocene]palladium(II) dichloromethane adduct as the catalyst, in the presence of a promoter such as copper (I) iodide, in a suitable inert solvent such as N,N-dimethylacetamide at a temperature of about 80° C.

In another embodiment, a compound of formula (VII-5) may be prepared according to the following scheme.

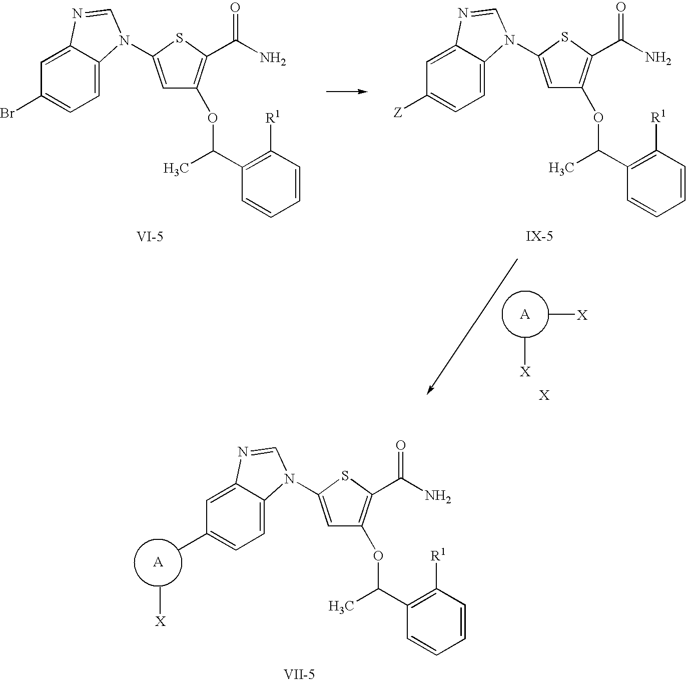

wherein each X is the same or different halo, Z is a a boronic acid, boronic ester, trifluoroborate salt (e.g., B(OH)₂ or pinacolboronate) stannane or zinc halide (e.g., zinc chloride), and all other variables are as defined above.

According to this process, a compound of formula (VII-5) may be prepared by a two-step process comprising:
a) reacting a compound of formula (VI-5) with a reagent suitable for installing the boronic acid, boronic ester, trifluoroborate salt, stannane or zinc halide, to prepare a compound of formula (IX-5); and
b) reacting the compound of formula (IX-5) with a compound of formula (X) to prepare a compound of formula (VII-5).

The reaction of the compound of formula (IX-5) with the haloarene of formula (X), may be carried out using palladium-catalyzed Suzuki, Stille, or Negishi palladium-catalyzed cross-coupling techniques using the methods as described above.

The compound of formula (IX-5) may be prepared by reaction of compound of formula (VI-5) with a reagent suitable for installing the boronic acid, boronic ester, trifluoroborate salt, stannane or zinc halide using techniques known in the art. For example, a compound of formula (VI-5) may be reacted with bis(pinacolato)diboron in the presence of a palladium source, optionally a phosphine ligand, and a base in a suitable inert solvent. Examples of suitable palladium sources include but are not limited to tris(dibenzylideneacetone)dipalladium (0), dichlorobis(triphenylphosphine)-palladium (II) or acetato(2′-di-t-butylphosphino-1,1′-biphenyl-2-yl)palladium (II). Examples of suitable phosphine ligands include but are not limited to 9,9-dimethyl-4,5-bis(diphenylphosphino)xanthene and triphenylphosphine. Examples of suitable bases include but are not limited to potassium acetate, cesium carbonate, sodium methoxide, and triethylamine. Examples of suitable inert solvents include but are not limited to toluene, N,N-dimethylformamide or 1,4-dioxane. The reaction may be carried out at a temperature of about 150° C., optionally in the microwave. Suitable reagents and reaction conditions for installation of a stannane or zinc halide will be readily apparent to those skilled in the art from the description above of the Stille and Negishi coupling reactions and the available literature.

A compound of formula (I-5) may be prepared more directly from a compound of formula (VI-5) by reacting the compound of formula (VI-5) with an appropriate aryl metal reagent of formula (VIII-A) under palladium catalyzed Suzuki, Stille or Negishi coupling conditions analogous to those described above for the preparation of a compound of formula (VII-5).

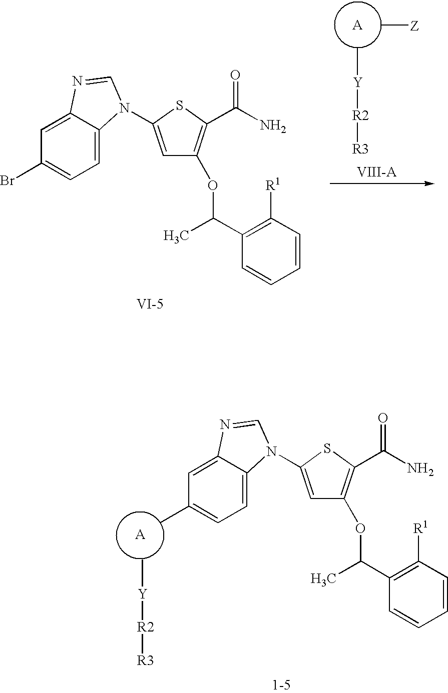

wherein Z is a a boronic acid, boronic ester, trifluoroborate salt (e.g., B(OH)₂ or pinacolboronate) stannane or zinc halide (e.g., zinc chloride), and all other variables are as defined above.

The compound of formula (VI-5) may be prepared by reacting a compound of formula (V-5) with ammonia. This reaction can be carried out in a molar solution of ammonia in an inert solvent and heated to a temperature of 70-100° C. Examples of suitable solvents for this reaction include but are not limited to methanol and dioxane.

A compound of formula (V-5) may be prepared by cyclizing the compound of formula (IV-5) with a suitable cyclizing agent (and optionally removing a protecting group if employed). Suitable cyclizing agents will be apparent to those skilled in the art of organic synthesis and include, for example triethyl orthoformate or trimethyl orthoformate, optionally in the presence of an acid catalyst, such as for example, pyridinium p-toluenesulfonate. In one embodiment, the cyclizing agent is triethyl orthoformate. Conveniently, the reaction of a compound of formula (IV-5) with the cyclization agent may be carried out neat or in a solvent such as methylene chloride, at a temperature of from about 25° C. to about 100° C. In one embodiment the reaction is carried out at ambient temperature.

A compound of formula (IV-5) may be prepared by reducing a compound of formula (III-5). The step of reducing a compound of formula (III-5) may be carried out using conventional reduction techniques suitable for such compounds. In particular, the reduction may be effected using conditions such as palladium on carbon under a hydrogen atmosphere, sulfided platinum on carbon under a hydrogen atmosphere, or iron powder in acetic acid. In one embodiment, the reduction may be effected using sulfided platinum on carbon under a hydrogen atmosphere. The reaction may be carried out in an inert solvent at either atmospheric or elevated pressure. Suitable inert solvents include but are not limited to ethanol, methanol, and ethyl acetate.

A compound of formula (III-5) may be prepared by reacting 3,5-dibromonitro-benzene with a compound of formula (II). This coupling reaction may be carried out using conventional Buchwald reaction conditions. Examples of suitable coupling reactions include but are not limited to palladium catalyzed cross-coupling conditions. Palladium catalyzed cross-coupling conditions include but are not limited to reacting 3,5-dibromonitrobenzene with the compound of formula (II) in the presence of a palladium source, optionally a phosphine ligand, and a base in a suitable inert solvent. Examples of suitable palladium sources include but are not limited to tris(dibenzylideneacetone)-dipalladium (0) or acetato(2′-di-t-butylphosphino-111′-biphenyl-2-yl)palladium (II). Examples of suitable phosphine ligands include but are not limited to 9,9-dimethyl-4,5-bis(diphenylphosphino)-xanthene. Examples of suitable bases include but are not limited to cesium carbonate, sodium methoxide, and triethylamine. Examples of suitable inert solvents include but are not limited to toluene or 1,4-dioxane. The reaction may be carried out at a temperature of between about room temperature and about 100° C. In one embodiment, the temperature is about 60° C. For a review of palladium-catalyzed cross-couplings of haloarenes and amines, see: Yang, B. H.; Buchwald, S. L. Journal of Organometallic Chemistry 1999, 576, 125-146. See also: Yin, J.; Zhao, M. M.; Huffman, M. A.; McNamara, J. M. Journal of Organic Chemistry 2002, 4, 3481-3484.

An enantiomerically enriched compound of formula (III-5) may be prepared directly through the use of enantiomerically enriched compound of formula (II-1).

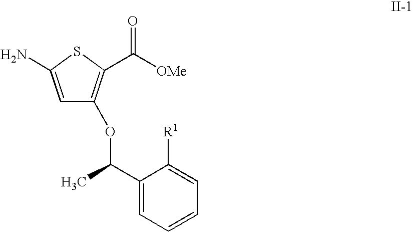

A compound of formula (II) may be prepared by reducing a compound of formula (XI) using conventional reduction techniques.

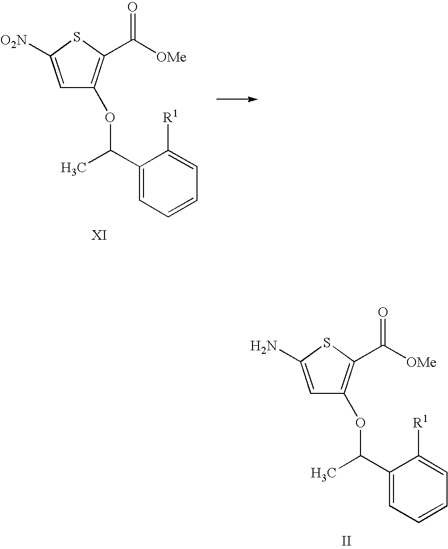

wherein all variables are as defined above.

Appropriate conditions for the reduction reaction will be apparent to those skilled in the art and include, for example, reducing agents, such as iron, in a suitable solvent, such as acetic acid. The reaction may be carried out with elevated temperatures, such as about 50° C.

Compounds of formula (XI) may be prepared by reacting a compound of formula (XII) with benzyl alcohol of formula (XIII).

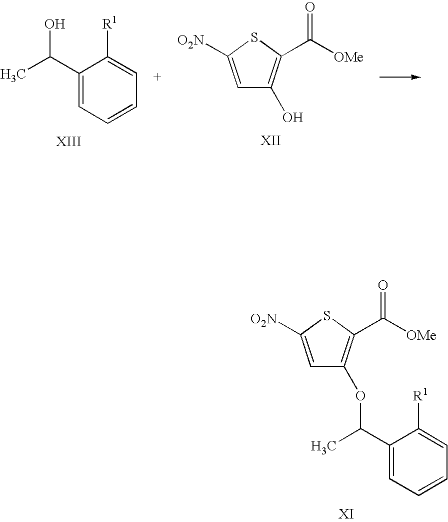

wherein all variables are as defined above.

This reaction may be carried out in an enantiomerically enriched fashion using conventional Mitsunobu techniques with inversion of absolute stereochemistry at the benzylic position. The reaction is carried out in an inert solvent under standard Mitsunobu conditions. See, Hughes, D. L., Org. React. 42:335-656 (1992); and Mitsunobu, O., Synthesis 1-28 (1981). Typically the compound of formula (XII), the compound of formula (XIII), a triarylphosphine, and a dialkyl azodicarboxylate are reacted together at room temperature, optionally using solid-supported reagents. Examples of suitable triarylphosphines include but are not limited to, triphenylphosphine, tri-tolylphosphine, and trimesitylphosphine. Examples of suitable dialkyl azodicarboxylates include but are not limited to, diethyl azodicarboxylate, diisopropyl azodicarboxylate, and di-tert-butyl azodicarboxylate. Examples of suitable inert solvents for this reaction include but are not limited to, tetrahydrofuran, dioxane, 1,2-dimethoxyethane, dichloromethane, and toluene.

This process may be used to prepare an enantiomerically enriched compound of formula (XI) and ultimately enantiomerically enriched compounds of formula (II-1) using techniques known to those skilled in the art.

Compounds of formula (XII) may be prepared in a manner analogous to the literature procedure (Barker, J. M.; Huddleston, P. R.; Wood, M. L.; Burkitt, S. A. Journal of Chemical Research (Miniprint) 2001, 1001-1022).

The enantiomerically enriched compounds of formula (XIII) are commercially available or either enantiomer can be prepared using conventional knowledge in the art.

In one particular embodiment, compounds of the invention wherein Ring A is 5- or 6-thiazole or 5- or 6-oxazole may be prepared by a process analogous to that described in Scheme 1 above. Scheme 2 below depicts the process specific for the preparation of compounds of formula (I-5A) wherein Ring A is a 5-oxazole or 5-thiazole. One skilled in the art will appreciate that the corresponding 6-oxazole and 6-thiazole analogs may be prepared using the same procedure with the appropriate starting material.

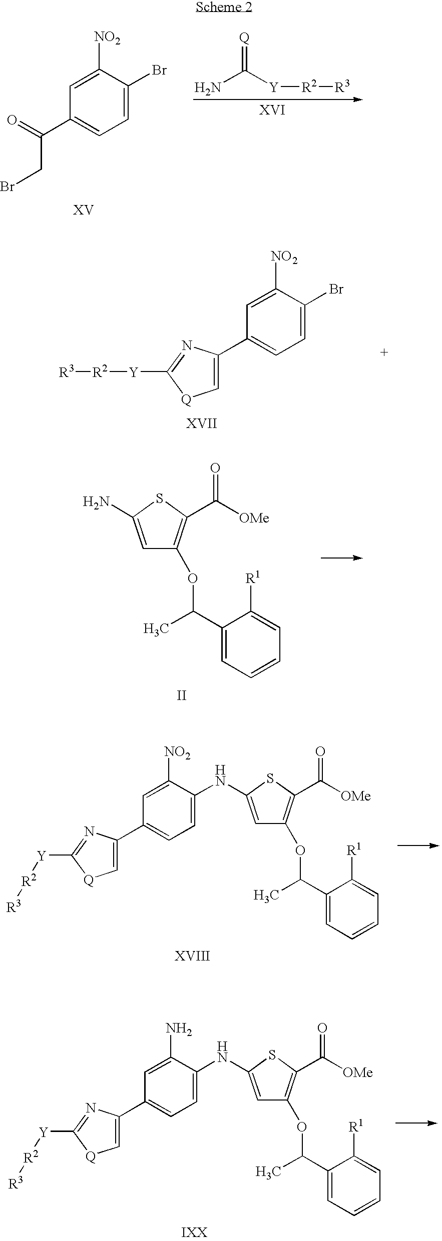

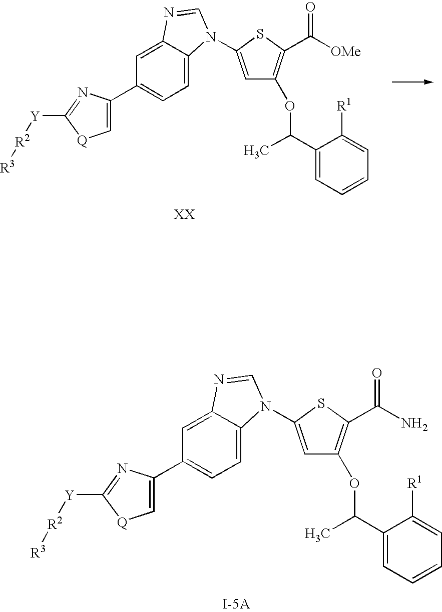

wherein:
Me is methyl; Q is O or S, with the proviso that for this embodiment of the invention, when Y is —O—, Q is S; and all other variables are as defined above.

As with Scheme 1, the foregoing reaction steps may be carried out in any suitable order and certain reaction steps may be most efficiently performed by installing protecting groups prior to the reaction, which are removed subsequently.

A compound of formula (I-5A) (i.e., a compound of formula (I) wherein Ring A is 5-oxazole or 5-thiazole and all other variables are as defined above), may be prepared by a process which comprises the steps of:
a) reacting a compound of formula (XV) with a compound of formula (XVI) to prepare a compound of formula (XVII);
b) reacting a compound of formula (XVII) with a compound of formula (II) to prepare a compound of formula (XVIII);
c) reducing the compound of formula (XVIII) to prepare a compound of formula (IXX);
d) cyclizing the compound of formula (IXX) to prepare a compound of formula (XX);
e) reacting the compound of formula (XX) with ammonia to prepare a compound of formula (I-5A);
f) optionally removing protecting groups;
g) optionally converting the compound of formula (I-5A) to a pharmaceutically acceptable salt thereof; and
h) optionally converting the compound of formula (I-5A) or a pharmaceutically acceptable salt thereof to a different compound of formula (I-5A) or a pharmaceutically acceptable salt thereof.

The foregoing steps b) through h) may be carried out in the manner described above in Scheme 1 for analogous reaction steps.

The compound of formula (XVII) may be prepared by reacting a compound of formula (XV) with a urea or thiourea of formula (XVI) in an inert solvent at a temperature from about room temperature to about 80° C. Suitable solvents include but are not limited to N,N-dimethylformamide, tetrahydrofuran, dioxane, toluene, benzene, 1,2-dimethoxyethane, and 1-methyl-2-pyrrolidinone, ethanol and isopropanol.

The compounds of formula (XV) and (XVI) are commercially available or may be prepared using conventional techniques. The compound of formula (XV-6):

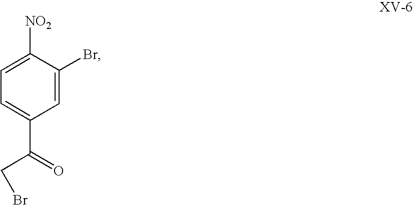

is also commercially available or may be prepared using conventional techniques, and may be used to prepare compounds of formula (I) wherein Ring A is 6-oxazole or 6-thiazole.

Compounds of the invention wherein Ring A is at either the 5- or 6-position of the benzimidazole may be prepared by the process outlined in Scheme 3 below.

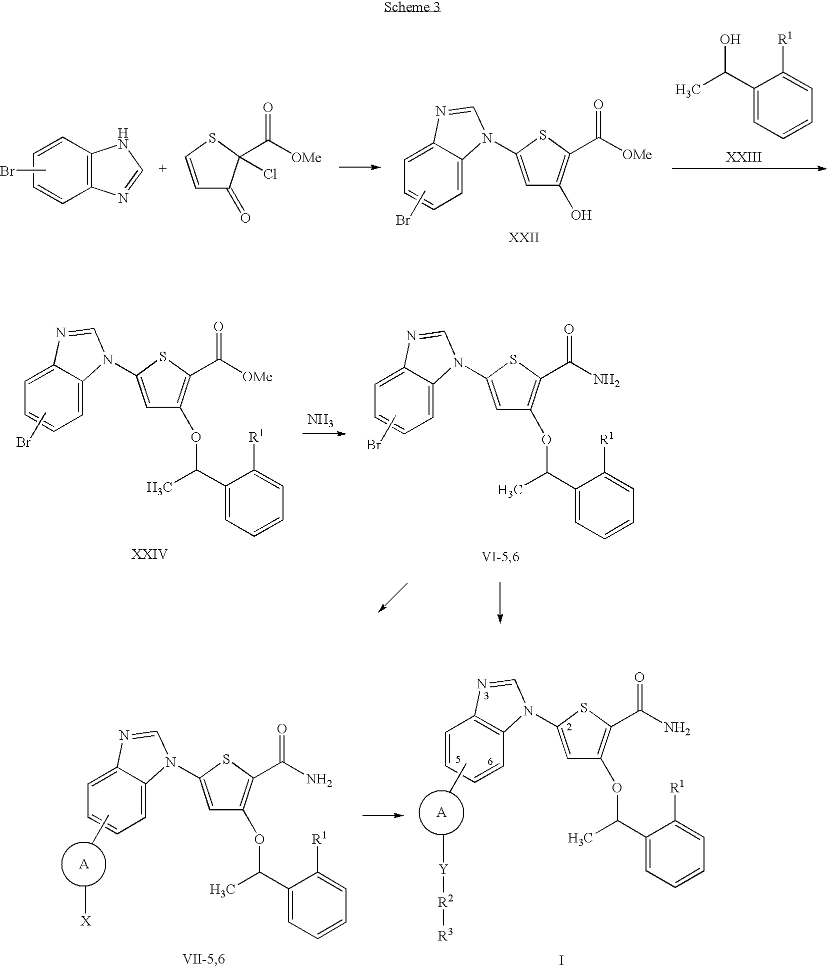

wherein Me is methyl, X is halogen and all other variables are as defined above and Br and Ring A are bound to the 5- or 6-position of the benzimidazole.

As with Scheme 1, the foregoing reaction steps may be carried out in any suitable order and certain reaction steps may be most efficiently performed by installing protecting groups prior to the reaction, which are removed subsequently. Similarly, at any point in the reaction a compound or intermediate may be separated into its regioisomers.

Generally, the process according to Scheme 3, for preparing compounds of the invention (all formulas and all variables having been defined above) comprises the steps of:
a) reacting 5-bromo-1H-benzimidazole with methyl 2-chloro-3-oxo-2,3-dihydro-2-thiophenecarboxylate to prepare a compound of formula (XXII);
b) reacting the compound of formula (XXII) with a compound of formula (XXIII) to prepare a compound of formula (XXIV);
c) reacting the compound of formula (XXIV) with ammonia to prepare a compound of formula (VI-5,6);
d) optionally separating the compound of formula (VI-5,6) into regioisomers of formula (VI-5) and (VI-6);
e) reacting the compound of formula (VI-5,6) or regioisomers of formula (VI-5) and (VI-6) with a reagent of formula (VIII) to prepare a compound of formula (VII-5,6) or regioisomers of formula (VII-5) and (VII-6);
f) reacting a compound of formula (VII-5,6) or regioisomers of formula (VII-5) and (VII-6) with a compound of formula (H—Y—R²—R³) to prepare a compound of formula (I) or regioisomers thereof;
g) optionally removing protecting groups;
h) optionally converting the compound of formula (I) to a pharmaceutically acceptable salt thereof; and
i) optionally converting the compound of formula (I) or a pharmaceutically acceptable salt thereof to a different compound of formula (I) or a pharmaceutically acceptable salt thereof.

Regio-separation may optionally be performed at any stage of the reaction. In particular, regio-separation may be performed either before or after reaction with ammonia to convert the ester to the corresponding amide. Thus, steps c) and d) may be performed in reverse order from that described above. Regio-separation may be carried out using conventional chromatographic separation techniques.

For the avoidance of doubt, the regio-isomers referred to include:

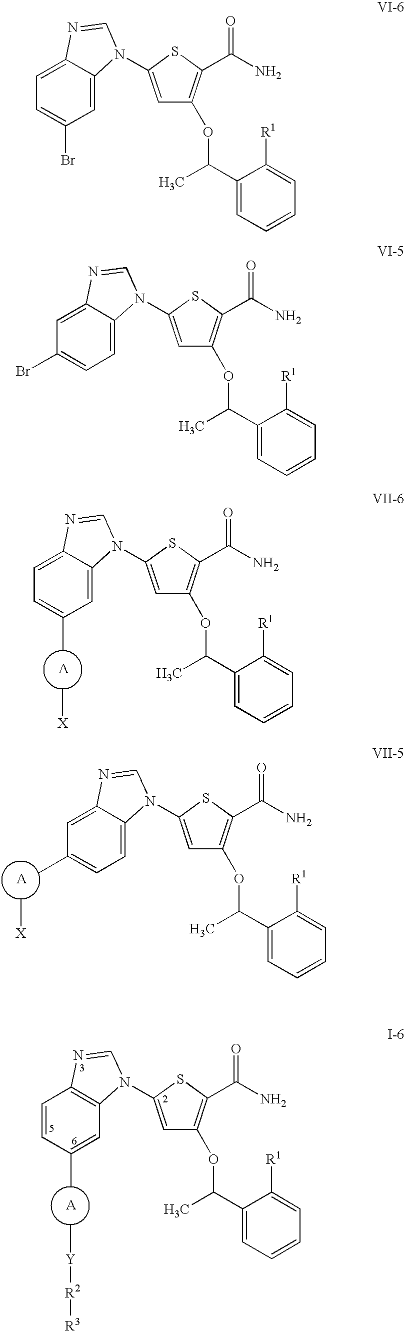

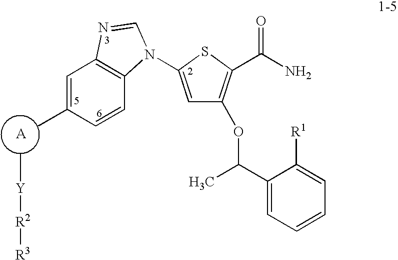

wherein all variables are as defined above. Each of the foregoing compounds constitute further aspects of the invention.

More specifically, a compound of formula (I) may be prepared from any of the compounds of formula (VI-5,6), (VII-5,6) or regioisomers thereof using the palladium-catalyzed coupling or palladium-catalyzed coupling and nucleophillic displacement reactions described above in Scheme 1. Thus a compound of formula (I) may be prepared by reacting a compound of formula (VII-5,6) (or either individual regioisomer) with a compound of formula (H—Y—R²—R³). A compound of formula (I) may also be prepared by reacting a compound of formula (VI-5,6) (or either individual regioisomer) with an aryl metal reagent of formula (VIII-A):

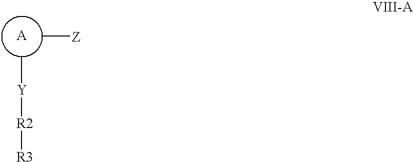

wherein Z is a a boronic acid, boronic ester, trifluoroborate salt, stannane or zinc halide, and all other variables are as defined above.

A compound of the formula (VI-5,6) may be prepared by reacting a compound of formula (XXIV) with ammonia in the manner described in Scheme 1 for the reaction of a compound of formula (V-5) with ammonia.

A compound of formula (XXIV) may be prepared by reacting a compound of formula (XXII) with a compound of formula (XXIII) under Mitsunobu reaction conditions.

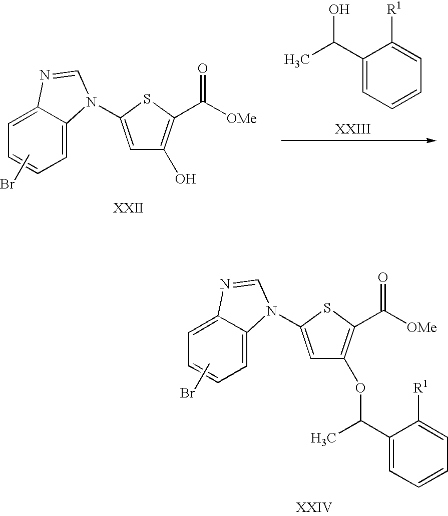

wherein all variables are as defined above and Br is bound to the 5- or 6-position of the benzimidazole.

The reaction is carried out in an inert solvent under standard Mitsunobu conditions. See, Hughes, D. L., Org. React. 42:335-656 (1992); and Mitsunobu, O., Synthesis 1-28 (1981). Typically the compound of formula (XXII), the compound of formula (XXIII), a triarylphosphine, and a dialkyl azodicarboxylate are reacted together at room temperature, optionally using solid-supported reagents. Examples of suitable triarylphosphines include but are not limited to, triphenylphosphine, tri-p-tolylphosphine, and trimesitylphosphine. Examples of suitable dialkyl azodicarboxylates include but are not limited to, diethyl azodicarboxylate, diisopropyl azodicarboxylate, and di-tert-butyl azodicarboxylate. Examples of suitable inert solvents for this reaction include but are not limited to, tetrahydrofuran, dioxane, 1,2-dimethoxyethane, dichloromethane, and toluene.

As will be apparent to those skilled in the art, use of the enantiomerically enriched compound of formula (XXIII-1)

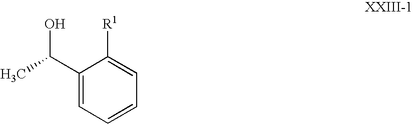

in the reaction with the compound of formula (XXII) will yield an enantiomerically enriched compound of formula (XXIV-1) which may be employed to yield the enantiomerically enriched compounds of formula (VI-5,6) and (VII-5,6) and ultimately the enantiomerically enriched compound of formula (I-1).

The compounds of formula (XXII) may be prepared by reaction of 5-bromo-1H-benzimidazole with methyl 2-chloro-3-oxo-2,3-dihydro-2-thiophenecarboxylate. Processes for the reaction are known to those skilled in the art. See, PCT Int. Appl. WO 2004073612. Such reactions are typically carried out in an inert solvent at room temperature. Examples of suitable inert solvents for this reaction include but are not limited to, chloroform, dichloromethane, tetrahydrofuran, dioxane, and toluene and mixtures of any of the foregoing with acetic acid (e.g., a mixture of chloroform and acetic acid). In one embodiment, the inert solvent is selected from dichloromethane, chloroform, tetrahydrofuran, diethyl ether, and toluene and a mixture of any of the foregoing and acetic acid (e.g. a mixture of chloroform and acetic acid).

The reaction may be carried out in the presence of one to five equivalents of the base additive. The base additive is believed to act as a scavenger for the hydrochloric acid generated during the reaction. Examples of suitable base additives for this reaction include but are not limited to sodium bicarbonate, triethylamine, sodium acetate, N-methylimidazole, pyridine, N-methylbenzimidazole and potassium carbonate. In one embodiment, the base additive is selected from sodium bicarbonate, triethylamine, sodium acetate, N-methylimidazole, pyridine and N-methylbenzimidazole. In one particular embodiment, the base additive is sodium bicarbonate. In one particular embodiment, the base additive is N-methylimidazole.

In another embodiment, a compound of formula (I) wherein Ring A is at the 5-position of the benzimidazole and Y is —N(R⁴)—C(O)— may be prepared according to the process depicted in Scheme 4.

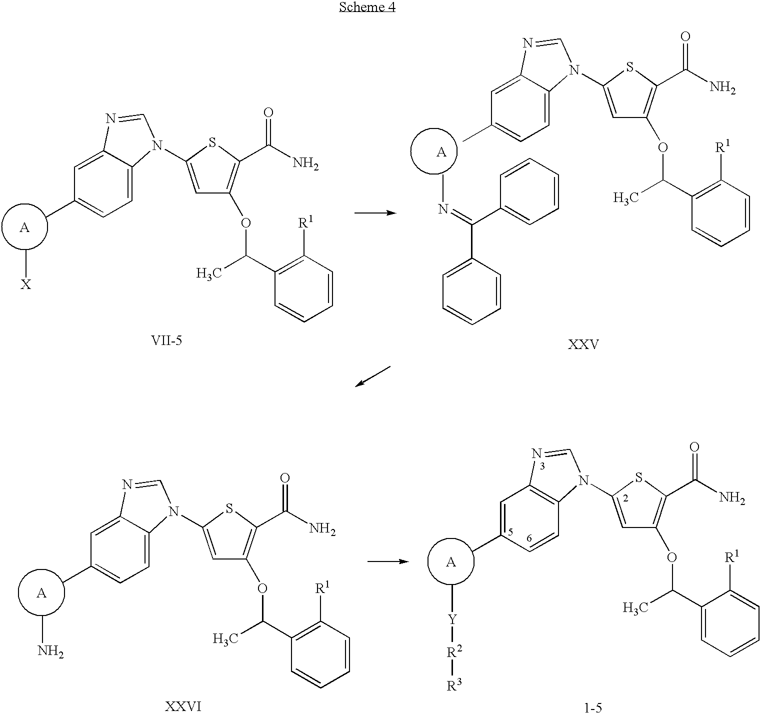

wherein X is halo and all other variables are as defined above.

According to this embodiment, a compound of formula (I) may be prepared by the steps comprising:
a) reacting the compound of formula (VII-5) under conventional cross-coupling reaction conditions to prepare a compound of formula (XXV);
b) hydrolyzing the compound of formula (XXV) to prepare a compound of formula (XXVI); and
c) reacting the compound of formula (XXVI) with a suitable carboxylic acid under standard amide coupling conditions to prepare a compound of formula (I-5);
d) optionally removing any protecting groups under standard deprotection conditions;
e) optionally converting the compound of formula (I-5) to a pharmaceutically acceptable salt thereof; and
f) optionally converting the compound of formula (I-5) or a pharmaceutically acceptable salt thereof to a different compound of formula (I-5) or a pharmaceutically acceptable salt thereof.

As with other described synthesis routes, the order of the foregoing steps is not critical to the practice of the present invention and the reaction steps may be carried out in any suitable order according to the skill in the art. It will be appreciated that the foregoing reaction may be carried out using the corresponding ester as the starting material and subsequently converting the ester to the amide by reaction with ammonia as described above. For example, the reaction with ammonia to convert the ester to the amide may be carried out before or after the reaction with the carboxylic acid. Additionally, it may be desirable to utilize protecting groups for one or more steps in the reaction. Techniques for the installation and removal of protecting groups are known the art.

A compound of formula (I-5) may be prepared by reacting a compound of formula (XXVI) with a suitable carboxylic acid of formula HO₂C—R²—R³ or HO₂C—R²—OC(O)—R²—R³ using conventional amide bond coupling conditions. This reaction can be carried out in an inert solvent using a variety of commercially available coupling reagents. Suitable coupling reagents include but are not limited to N,N-dicyclohexylcarbodiimide, 1-(3-dimethylaminopropyl)-3-ethylcarbodiimide hydrochloride, 1,1′-carbonyldiimidazole, 2-(7-aza-1H-benzotriazole-1-yl)-1,1,3,3-tetramethyluronium hexafluorophosphate and benzotriazol-1-yloxytris(dimethyl-amino)phosphonium hexafluorophosphate. Other suitable coupling reagents will be readily apparent to those skilled in the art. The carboxylic acid optionally may be converted into the corresponding acid chloride and subsequently treated with ammonia. Suitable reagents for the reaction of such acid chlorides include but are not limited to oxalyl chloride, thionyl chloride, and 1-chloro-N,N,2-trimethyl-1-propenylamine. Base may be optionally added to the coupling reaction. The reaction may optionally require heating to a temperature of from about 40° C. to about 100° C. Suitable bases include but are not limited to trialkylamines, pyridine, and 4-(dimethylamino)pyridine. Examples of suitable solvents for this reaction include but are not limited to methanol, dichloromethane, chloroform, benzene, toluene, N,N-dimethylformamide and dichloroethane. This reaction may be followed by a deprotection reaction using conventional techniques. In the embodiment where a t-butylcarbamate is employed, the final step involves removal of this group using conventional techniques. See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991.

A compound of formula (XXVI) may be prepared from a compound of formula (XXV) by reaction with a strong acid in a suitable organic solvent using conventional acidic deprotection techniques. Suitable acids used in such transformations include but are not limited to hydrochloric acid. Suitable solvents for such transformations include but are not limited to tetrahydrofuran and 1,4-dioxane. See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991.

A compound of formula (XXV) may be prepared by Buchwald coupling of a compound of formula (VII-5) with benzophenone imine in an inert solvent in the presence of Pd (0) catalyst, base and phosphine ligand. Examples of suitable coupling reactions include but are not limited to palladium catalyzed cross-coupling conditions. Palladium catalyzed cross-coupling conditions include but are not limited to reacting 3,5-dibromonitrobenzene with the compound of formula (II) in the presence of a palladium source, optionally a phosphine ligand, and a base in a suitable inert solvent. Examples of suitable palladium sources include but are not limited to tris(dibenzylideneacetone)-dipalladium (0) or acetato(2′-di-t-butylphosphino-1,1′-biphenyl-2-yl)palladium (II). Examples of suitable phosphine ligands include but are not limited to 9,9-dimethyl-4,5-bis(diphenylphosphino)-xanthene. Examples of suitable bases include but are not limited to cesium carbonate, sodium methoxide, and triethylamine. Examples of suitable inert solvents include but are not limited to toluene or 1,4-dioxane. The reaction may be carried out at a temperature of between about room temperature and about 100° C. In one embodiment, the temperature is about 90° C.

As will be apparent to those skilled in the art, a compound of formula (I) may be converted into another compound of formula (I) using conventional techniques known to those skilled in the art. For example, a compound of formula (I) wherein Y—R²—R³ is N(H)—(CH₂)₂—OH may be converted to a compound of formula (I) wherein Y—R²—R³ is N(H)—(CH₂)₂—OC(O)—(CH₂)₂—NH₂ using conventional amide bond coupling techniques described hereinabove.

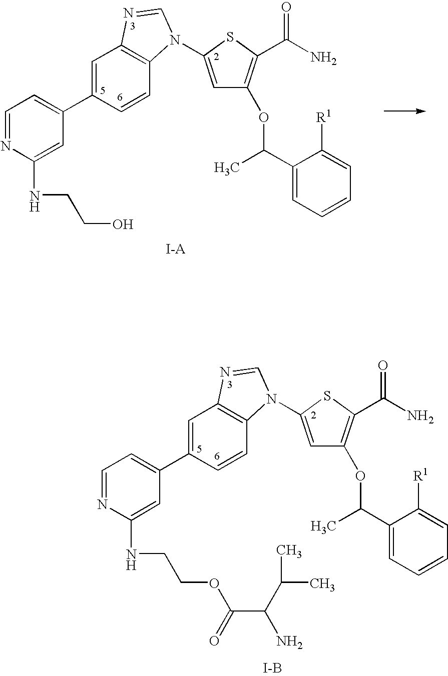

wherein R¹ is as defined above.

Based upon this disclosure and the examples contained herein one skilled in the art can readily convert a compound of formula (I) or (I-1) or a pharmaceutically acceptable salt thereof into another compound of formula (I) or (I-1) or a pharmaceutically acceptable salt thereof.

The following abbreviations as employed in the examples, have the recited meanings.
g gram(s)
mg milligram(s)
mol mole(s)
mmol millimole(s)
N normal
L liter(s)
mL milliliter(s)
μL microliter(s)
h hour(s)
min minute(s)
° C. degrees Centigrade
HCl hydrochloric acid
DCM dichloromethane
CHCl₃ chloroform
MeOH methanol
EtOH ethanol
i-PrOH isopropanol
EtOAc ethyl acetate
THF tetrahydrofuran
TFA trifluoroacetic acid
DMA N,N-dimethylacetamide
DMF N,N-dimethylformamide
NH₄Cl ammonium chloride
MgSO₄ magnesium sulfate
NaOH sodium hydroxide
NaHCO₃ sodium bicarbonate
Na₂CO₃ sodium carbonate
K₂CO₃ potassium carbonate
Cs₂CO₃ cesium carbonate
Na₂SO₄ sodium sulfate
N₂ nitrogen
H₂ hydrogen
rt room temperature
Cl₂Pd(dppf) dichloro[1,1′-bis(diphenylphosphino)ferrocene]palladium(II)
XANTPHOS (4,5-Bis(diphenylphosphino)-9,9-dimethylxanthene) is a commercially available catalyst, from Aldrich
SFC supercritical fluid chromatography
TLC thin layer chromatography.
ee enantiomeric excess

Reagents are commercially available or are prepared according to procedures in the literature. In the following structures, “Me” refers to the group —CH₃.

All references to “ether” are to diethyl ether; brine refers to a saturated aqueous solution of NaCl. Unless otherwise indicated, all temperatures are expressed in ° C. (degrees Centigrade). All reactions are conducted under an inert atmosphere at rt unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APIiii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution or mass spectrometry (electrospray or AP). Flash column chromatography was performed on silica gel (230-400 mesh, Merck) or using automated silica gel chromatography (Isco, Inc. Sq 16× or 100 sg Combiflash).

Reported HPLC retention times (RT) were obtained on a Waters 2795 instrument attached to a Waters 996 diode array detector reading 210-500 nm. The column used was a Synergi Max-RP (50×2 mm) model #00B-4337-B0. Solvent gradient was 15% MeOH:water to 100% MeOH (0.1% formic acid) over 6 min. Flow rate was 0.8 mL/min. Injection volume was 3 μL.

### INTERMEDIATE EXAMPLE 1

### Methyl 5-amino-3-({(1R)-1-[2-(trifluoromethyl)phenyl]ethyl}oxy)-2-thiophenecarboxylate

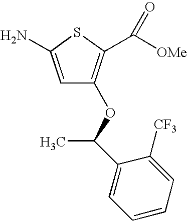

### Step A—Methyl 5-nitro-3-({(1R)-1-[2-(trifluoromethyl)phenyl]ethyl}oxy)-2-thiophenecarboxylate

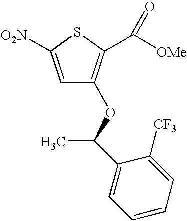

A slurry of polymer-supported triphenylphosphine (62.36 g, 2.21 mmol/g, 137.8 mmol) in DCM (1.0 L) was stirred at rt for 10 min. The mixture was cooled to 0° C. Methyl 3-hydroxy-5-nitro-2-thiophenecarboxylate (20.00 g, 98.44 mmol), which may be prepared in a manner analogous to the literature procedure (Barker, J. M.; Huddleston, P. R.; Wood, M. L.; Burkitt, S. A. Journal of Chemical Research (Miniprint) 2001, 1001-1022) was added, followed by (1S)-1-[2-(trifluoromethyl)phenyl]ethanol (26.20 g, 137.8 mmol) and di-tert-butyl azodicarboxylate (31.73 g, 137.8 mmol). The reaction mixture was stirred at rt for 21.25 h and then was filtered through a fritted funnel and concentrated. The residue was treated with 4 N HCl in 1,4-dioxane (300 mL) and stirred at rt for 3 h. The mixture was then quenched by addition of 3 N NaOH (300 mL) and saturated aqueous sodium bicarbonate (200 mL). The mixture was extracted with DCM (3×250 mL). The combined organic fractions were dried over MgSO₄, filtered, and concentrated onto silica gel. Purification by column chromatography (0 to 25% EtOAc:hexanes) provided 36.08 g (98%) of the title compound as yellow oil. ¹H NMR (300 MHz, CDCl₃): δ 7.82 (d, 1H, J=7.8 Hz), 7.68 (d, 1H, J=7.8 Hz), 7.59 (t, 1H, J=7.4 Hz), 7.46 (s, 1H), 7.42 (t, 1H, J=7.6 Hz), 5.77 (q, 1H, J=6.1 Hz), 3.94 (s, 3H), 1.74 (d, 3H, J=6.1 Hz).

### Step B—Methyl 5-amino-3-({(1R)-1-[2-(trifluoromethyl)phenyl]ethyl}oxy)-2-thiophenecarboxylate (title compound)

To a flask equipped with a temperature probe, an overhead mechanical stirrer, a reflux condenser, and an addition funnel was added iron powder (26.84 g, 480.6 mmol) and AcOH (130 mL). The iron/AcOH slurry was stirred mechanically and heated to an internal temperature of 50° C. To the addition funnel was added a solution of methyl 5-nitro-3-({(1R)-1-[2-(trifluoromethyl)phenyl]ethyl}oxy)-2-thiophenecarboxylate (36.08 g, 96.13 mmol) in AcOH (160 mL). The solution in the addition funnel was then added dropwise to the iron/AcOH slurry at a rate such that the internal temperature was maintained at <60° C. (2.5 h total addition time). The reaction mixture was cooled to rt, diluted with DCM (500 mL), and then quenched by addition of 6 N NaOH (750 mL) and saturated aqueous sodium bicarbonate (200 mL). The entire mixture was then filtered through a pad of Celite to remove insoluble material, rinsing the Celite with additional DCM (250 mL). The aqueous and organic fractions were separated. The aqueous fraction was extracted with EtOAc (2×400 mL). The organic fractions were combined, dried over MgSO₄, filtered, and concentrated to afford 30.66 g (92%) of the title compound as an orange solid. ¹H NMR (300 MHz, CDCl₃): δ 7.89 (d, 1H, J=7.7 Hz), 7.62 (d, 1H, J=7.7 Hz), 7.56 (t, 1H, J=7.7 Hz), 7.36 (t, 1H, J=7.7 Hz), 5.72 (s, 1H), 5.65 (q, 1H, J=6.3 Hz), 4.26 (br s, 2H), 3.80 (s, 3H), 1.66 (d, 3H, J=6.3 Hz); MS (APCI): 368.00 [M+Na]⁺.

### INTERMEDIATE EXAMPLE 2

### Methyl 5-amino-3-{[(1R)-1-(2-chlorophenyl)ethyl]-oxy}-2-thiophenecarboxylate

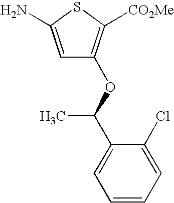

### Step A—Methyl 3-{[(1R)-1-(2-chlorophenyl)ethyl]oxy}-5-nitro-2-thiophenecarboxylate

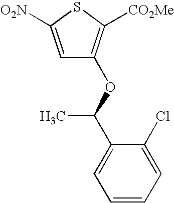

The title compound was prepared from methyl 3-hydroxy-5-nitro-2-thiophenecarboxylate and (1S)-1-(2-chlorophenyl)ethanol by a procedure analogous to Intermediate Example 1, Step A. ¹H NMR (400 MHz, DMSO-d₆): δ 7.96 (s, 1H), 7.65 (dd, 1H, J=1.7, 7.8 Hz), 7.47 (dd, 1H, J=1.5, 7.7 Hz), 7.40 (dt, 1H, J=1.3, 7.5 Hz), 7.34 (dt, 1H, J=1.9, 7.5 Hz), 5.98 (q, 1H, J=6.0 Hz), 3.85 (s, 3H), 1.59 (d, 3H, J=6.2 Hz).

### Step B—Methyl 5-amino-3-{[(1R)-1-(2-chlorophenyl)ethyl]oxy}-2-thiophenecarboxylate (title compound)

The title compound was prepared from methyl 3-{[(1R)-1-(2-chlorophenyl)ethyl]oxy}-5-nitro-2-thiophenecarboxylate by a procedure analogous to Intermediate Example 1, Step B. ¹H NMR (400 MHz, DMSO-d₆): δ 7.54 (dd, 1H, J=1.8, 7.9 Hz), 7.45 (dd, 1H, J=1.4, 7.7 Hz), 7.37 (dt, 1H, J=1.4, 7.7 Hz), 7.31 (dt, 1H, J=1.8, 7.6 Hz), 6.76 (br s, 2H), 5.57 (q, 1H, J=6.2 Hz), 5.49 (s, 1H), 3.63 (s, 3H), 1.51 (d, 3H, J=6.4 Hz); MS (ESI): 334.03 [M+Na]⁺.

### EXAMPLE 1

### 5-[5-(2-{[2-(Dimethylamino)ethyl]amino}-4-pyridinyl)-1H-benzimidazol-1-yl]-3-({(1R)-1-[2-(trifluoromethyl)phenyl]ethyl}oxy)-2-thiophenecarboxamide

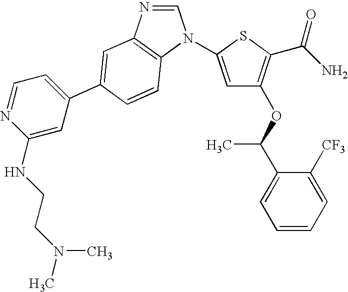

### Step A—Methyl 5-[(4-bromo-2-nitrophenyl)amino]-3-({(1R)-1-[2-(trifluoromethyl)phenyl]ethyl}oxy)thiophene-2-carboxylate

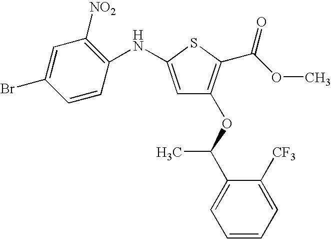

To a stirred mixture of methyl 5-amino-3-({(1R)-1-[2-(trifluoromethyl)phenyl]ethyl}oxy)-2-thiophenecarboxylate (7.86 g, 22.7 mmol), 2,5-dibromonitrobenzene (6.18 g, 22.0 mmol), tris(dibenzylideneacetone)dipalladium (0) (0.42 g, 0.46 mmol), and 4,5-bis(diphenylphosphino)-9,9 dimethylxanthene (0.59 g, 1.1 mmol) in toluene (175 mL) under nitrogen was added cesium carbonate (35.0 g, 88 mmol). The reaction mixture was stirred at 60° C. for 2 h after which time the mixture was cooled slightly and partitioned between EtOAc and water. The water was extracted with EtOAc again and the organics were combined, dried over MgSO₄ and filtered. The filtrate was concentrated onto silica gel. Purification by column chromatography (10 to 50% EtOAc:hexanes) provided 4.77 g (40%) of the title compound as a red foam. ¹H NMR (400 MHz, CDCl₃): δ 9.46 (s, 1H), 8.31 (d, 1H, J=2.4 Hz), 7.88 (s, 1H, J=8.0 Hz), 7.61 (m, 2H), 7.47 (m, 2H), 7.10 (d, 1H, J=8.8 Hz), 6.42 (s, 1H), 5.70 (m, 1H), 3.86 (s, 3H), 1.71 (d, 3H, J=6.4 Hz).

### Step B—Methyl 5-[(2-amino-4-bromophenyl)amino]-3-({(1R)-1-[2-(trifluoromethyl)phenyl]ethyl}oxy)thiophene-2-carboxylate

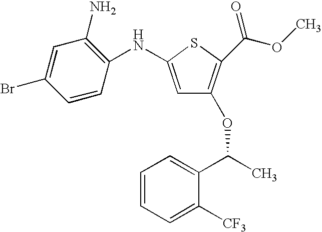

Methyl 5-[(4-bromo-2-nitrophenyl)amino]-3-({(1R)-1-[2-(trifluoromethyl)phenyl]-ethyl}oxy)thiophene-2-carboxylate (4.77 g, 8.75 mmol) and sulfided platinum (5 wt % on carbon, 1.80 g, 0.44 mmol) were stirred in EtOAc (100 mL) under 25 psi of hydrogen for 1 h after which time the mixture was filtered and concentrated to give the title compound as a yellow foam (4.50 g crude product, 100% yield). MS (ESI): 516.0 [M]⁺.

### Step C—Methyl 5-(5-bromo-1H-benzimidazol-1-yl)-3-({(1R)-1-[2-(trifluoromethyl)-phenyl]ethyl}oxy)thiophene-2-carboxylate

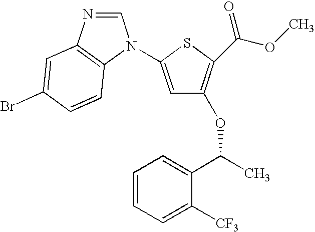

Methyl 5-[(2-amino-4-bromophenyl)amino]-3-({(1R)-1-[2-(trifluoromethyl)phenyl]-ethyl}oxy)thiophene-2-carboxylate (4.50 g crude product) and a catalytic amount of pyridinium p-toluenesulfonate (100 mg) were stirred in triethyl orthoformate (10 mL) at rt overnight after which time the reaction mixture was neutralized with solid sodium bicarbonate and partitioned between EtOAc and water. The aqueous layer was extracted twice with EtOAc. The organics were combined, dried over MgSO₄, filtered and concentrated to give 4.50 g of the desired crude product as a foam. ¹H NMR (400 MHz, CDCl₃): δ 7.90 (m, 3H), 7.61 (m, 2H), 7.41 (m, 2H), 7.28 (d, 1H, J=8.4 Hz), 6.72 (s, 1H), 5.78 (m, 1H), 3.90 (s, 3H), 1.75 (d, 3H, J=6.4 Hz). MS (ESI): 524.9 [M]⁺.

### Step D—5-(5-Bromo-1H-benzimidazol-1-yl)-3-({(1R)-1-[2-(trifluoromethyl)phenyl]-ethyl}oxy)thiophene-2-carboxamide

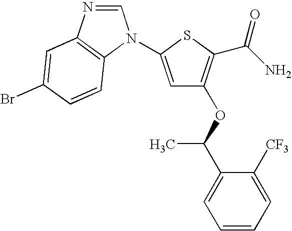

The title compound was prepared from methyl 5-(5-bromo-1H-benzimidazol-1-yl)-3-({(1R)-1-[2-(trifluoromethyl)phenyl]ethyl}oxy)thiophene-2-carboxylate (4.0 g, 7.61 mmol) and 100 mL of 7 N ammonia in MeOH heating in a sealed vessel at 80° C. for 48 h. The mixture was cooled to rt and filtered to give a yellow solid. The remaining filtrate was purified by column chromatography (50 to 100% EtOAc in hexanes), and the resulting solid was combined with the above yellow solid to give 3.30 g (85%) of the title compound. ¹H NMR (400 MHz, CDCl₃): δ 7.94 (d, 1H, J=1.6 Hz), 7.89 (s, 1H), 7.66 (m, 3H), 7.43 (m, 2H), 7.28 (m, 1H), 7.17 (s, 1H), 6.61 (s, 1H), 5.83 (s, 1H), 5.78 (m, 1H), 1.79 (d, 3H, J=6.4 Hz). MS (ESI): 511.0 [M+H]⁺.

### Step E—5-[5-(2-Fluoro-4-pyridinyl)-1H-benzimidazol-1-yl]-3-({(1R)-1-[2-(trifluoromethyl)phenyl]ethyl}oxy)-2-thiophenecarboxamide

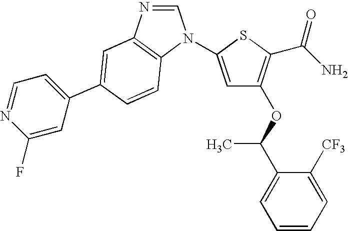

2-Fluoropyridine-4-boronic acid (0.497 g, 3.53 mmol), 5-(5-bromo-1H-benzimidazol-1-yl)-3-({(1R)-1-[2-(trifluoromethyl)phenyl]ethyl}oxy)thiophene-2-carboxamide (1.50 g, 2.94 mmol), and sodium carbonate (1 N in water, 10.3 mL) were combined in N,N-dimethylacetamide (30 mL). 1,1′-bisdiphenylphosphino-ferrocene dichloropalladium (II) (0.24 g, 0.3 mmol) was added and the reaction mixture was stirred under nitrogen while heating at 80° C. for 1.5 h. The mixture was then cooled and partitioned between DCM and water. The aqueous layer was extracted with DCM. The combined organics were dried over MgSO₄, concentrated onto silica gel and purified by column chromatography (0 to 50% EtOAc:hexanes) to afford 1.52 g (97%) of the title compound as a light tan solid after trituration in diethyl ether. ¹H NMR (400 MHz, CDCl₃): δ 8.29 (d, 1H, J=5.3 Hz), 8.12 (s, 2H), 7.68 (m, 4H), 7.51 (m, 3H), 7.21 (s, 1H), 7.18 (s, 1H), 6.71 (s, 1H), 5.85 (m, 2H), 1.82 (d, 3H, J=5.9 Hz); MS (ESI): 527.0 [M+H]⁺.

### Step F—5-[5-(2-{[2-(Dimethylamino)ethyl]amino}-4-pyridinyl)-1H-benzimidazol-1-yl]-3-({(1R)-1-[2-(trifluoromethyl)phenyl]ethyl}oxy)-2-thiophenecarboxamide (title compound)

A neat mixture of 5-[5-(2-fluoro-4-pyridinyl)-1H-benzimidazol-1-yl]-3-({(1R)-1-[2-(trifluoromethyl)phenyl]ethyl}oxy)-2-thiophenecarboxamide (0.248 g, 0.471 mmol) and N,N-dimethyl-1,2-ethanediamine (1.5 mL, 14.1 mmol) was heated in a Personal Chemistry microwave at 180° C. for 40 min after which time the mixture was diluted with DCM and washed with water. The aqueous phase was extracted with DCM and the organics were combined, dried over MgSO₄ and concentrated onto silica gel. Purification by column chromatography (10-90% 90/9/1 DCM/MeOH/ammonium hydroxide:DCM) provided 0.200 g (71%) of the title compound as a yellow solid. ¹H NMR (400 MHz, CDCl₃): δ 8.15 (d, 1H, J=5.3 Hz), 8.03 (d, 1H, J=1.3 Hz), 7.96 (s, 1H), 7.73 (d, 1H, J=8.0 Hz), 7.70 (d, 1H, J=7.9 Hz), 7.64 (t, 1H, J=7.6 Hz), 7.57 (dd, 1H, J=8.6, 1.5 Hz), 7.48 (m, 2H), 7.21 (br s, 1H), 6.83 (dd, 1H, J=5.5, 1.3 Hz), 6.66 (m, 2H), 6.03 (br s, 1H), 5.87 (q, 1H, J=6.3 Hz), 5.21 (t, 1H, J=4.7 Hz), 3.44 (q, 2H, J=5.6 Hz), 2.60 (t, 2H, J=6.0 Hz), 2.30 (s, 6H), 1.81 (d, 3H, J=6.2 Hz); MS (ESI): 595.1 [M+H]⁺.

### EXAMPLE 2

### 5-[5-(2-{[2-(Methylamino)ethyl]amino}-4-pyridinyl)-1H-benzimidazol-1-yl]-3-({(1R)-1-[2-(trifluoromethyl)phenyl]ethyl}oxy)-2-thiophenecarboxamide

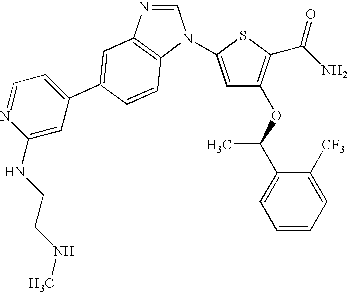

### Step A—1,1-Dimethylethyl {2-[(4-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]ethyl}methylcarbamate

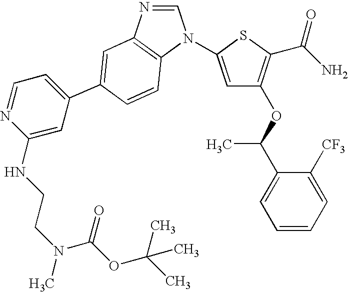

A neat mixture of 5-[5-(2-fluoro-4-pyridinyl)-1H-benzimidazol-1-yl]-3-({(1R)-1-[2-(trifluoromethyl)phenyl]ethyl}oxy)-2-thiophenecarboxamide (0.050 g, 0.09 mmol) (prepared according to Example 1, Step E) and 1,1-dimethylethyl (2-aminoethyl)methylcarbamate (0.6 mL) was heated in a sealed tube at 110° C. for 14 h after which time the mixture was diluted with EtOAc and washed with water. The aqueous phase was extracted twice with EtOAc and concentrated onto silica gel. Purification by column chromatography (10-70% 90/9/1 DCM/MeOH/ammonium hydroxide:DCM) provided 0.055 g (86%) of the title compound as a foam. MS (ESI): 681.3 [M+H]⁺.

### Step B—5-[5-(2-{[2-(Methylamino)ethyl]amino}-4-pyridinyl)-1H-benzimidazol-1-yl]-3-({(1R)-1-[2-(trifluoromethyl)phenyl]ethyl}oxy)-2-thiophenecarboxamide (title compound)

A solution of 1,1-dimethylethyl {2-[(4-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]ethyl}methylcarbamate (0.055 g, 0.08 mmol) and TFA (0.5 mL) was stirred in DCM (2 mL) at rt overnight. Excess solvents were removed in vacuo and the reaction mixture was concentrated onto silica gel. Purification by column chromatography (10-90% 90/9/1 DCM/MeOH/ammonium hydroxide:DCM) provided 0.034 g (72%) of the title compound as a light yellow solid. ¹H NMR (400 MHz, CDCl₃): δ 8.12 (d, 1H, J=5.3 Hz), 7.99 (s, 1H), 7.93 (s, 1H), 7.71 (d, 1H, J=8.1 Hz), 7.68 (d, 1H, J=7.9 Hz), 7.62 (t, 1H, J=7.5 Hz), 7.53 (dd, 1H, J=8.6, 1.3 Hz), 7.47-7.42 (m, 2H), 7.20 (brs, 1H), 6.82 (dd, 1H, J=5.3, 1.1 Hz), 6.65 (s, 1H), 6.12 (br s, 1H), 5.85 (q, 1H, J=6.2 Hz), 5.10 (t, 1H, J=5.4 Hz), 3.51 (q, 2H, J=5.6 Hz), 2.93-2.90 (m, 2H), 2.49 (s, 3H), 1.96 (s, 1H), 1.79 (d, 3H, J=6.2 Hz); MS (AP): 581.2 [M+H]⁺.

### EXAMPLE 3

### 5-(5-{2-[(N,N-dimethylglycyl)amino]-4-pyridinyl}-1H-benzimidazol-1-yl)-3-({(1R)-1-[2-(trifluoromethyl)phenyl]ethyl}oxy)-2-thiophenecarboxamide

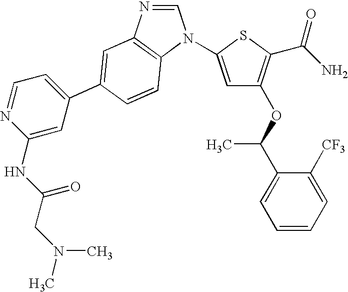

### Step A—Methyl 5-[5-(2-chloro-4-pyridinyl)-1H-benzimidazol-1-yl]-3-({(1R)-1-[2-(trifluoromethyl)phenyl]ethyl}oxy)-2-thiophenecarboxylate

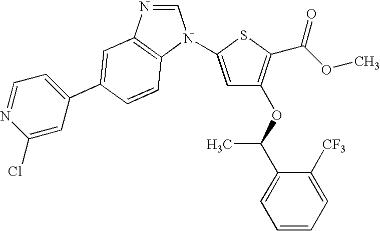

2-Chloro-4-(4,4,5,5-tetramethyl-1,3,2-dioxaborolan-2-yl)-pyridine (0.501 g, 2.09 mmol), methyl 5-(5-bromo-1H-benzimidazol-1-yl)-3-({(1R)-1-[2-(trifluoromethyl)phenyl]ethyl}oxy)thiophene-2-carboxylate (1.00 g, 1.90 mmol) (prepared according to Example 1, Step C), and sodium carbonate (1 N in water, 7.5 mL) were combined in N,N-dimethylacetamide (25 mL). 1,1′-bisdiphenyl-phosphinoferrocene dichloropalladium (II) (0.233 g, 0.209 mmol) was added and the reaction mixture was stirred under nitrogen while heating at 80° C. for 1 h. The mixture was then cooled and partitioned between 5:1 DCM:MeOH and saturated aqueous sodium bicarbonate. The organic layer was washed twice with brine, dried over sodium sulfate, and concentrated onto Celite. Purification by column chromatography (10 to 100% Jan. 9, 1990 ammonium hydroxide/MeOH/DCM: DCM) provided 0.354 g (33%) of the title compound as an off-white solid. ¹H NMR (400 MHz, CDCl₃): δ 8.43 (d, 1H, J=5.3 Hz), 8.04 (d, 2H, J=13.9 Hz), 7.90 (d, 1H, J=7.9 Hz), 7.66 (d, 1H, J=7.9 Hz), 7.58 (m, 4H), 7.47 (dd, 1H, J=1.1, 5.1 Hz), 7.41 (t, 1H, J=7.6 Hz), 6.78 (s, 1H), 5.81 (q, 1H, J=6.2 Hz), 3.92 (s, 3H), 1.77 (d, 3H, J=6.2 Hz); MS (ESI): 558.1 [M+H]⁺.

### Step B—Methyl 5-(5-{2-[(diphenylmethylidene)amino]-4-pyridinyl}-1H-benzimidazol-1-yl)-3-({(1R)-1-[2-(trifluoromethyl)phenyl]ethyl}oxy)-2-thiophenecarboxylate

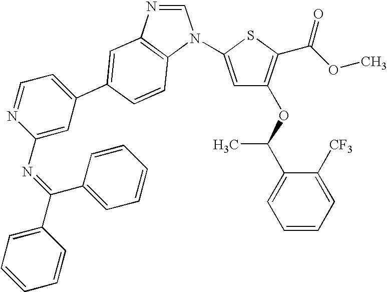

To a degassed solution of methyl 5-[5-(2-chloro-4-pyridinyl)-1H-benzimidazol-1-yl]-3-({(1R)-1-[2-(trifluoromethyl)phenyl]ethyl}oxy)-2-thiophenecarboxylate (0.250 g, 0.45 mmol, combined material from multiple iterations of Example 3 step A) and benzophenone imine (0.09 mL, 0.54 mmol) in 1,4-dioxane (2 mL), cesium carbonate (367 mg, 1.13 mmol) was added followed by 4,5-bis(diphenylphosphino)-9,9-dimethyl-xanthene (10.0 mg, 0.018 mmol) and tris(dibenzylidineacetone)dipalladium (0) (8.0 mg, 0.009 mmol). The reaction mixture was heated to 90° C. for 24 h. The reaction mixture was then diluted with EtOAc, concentrated onto silica gel and purified by column chromatography (0 to 60% EtOAc:hexanes) to afford 0.255 g (83%) of the title compound as an off-white solid. MS (ESI): 703.3 [M+H]⁺.

### Step C—Methyl 5-[5-(2-amino-4-pyridinyl)-1H-benzimidazol-1-yl]-3-({(1R)-1-[2-(trifluoromethyl)phenyl]ethyl}oxy)-2-thiophenecarboxylate

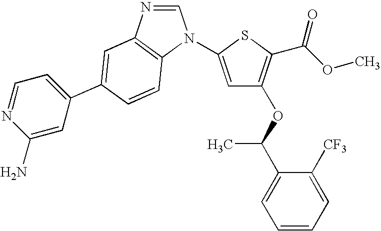

To a solution of methyl 5-(5-{2-[(diphenylmethylidene)amino]-4-pyridinyl}-1H-benzimidazol-1-yl)-3-({(1R)-1-[2-(trifluoromethyl)phenyl]ethyl}oxy)-2-thiophenecarboxylate (0.160 mg, 0.228 mmol) in THF (6 mL), HCl (2N, aqueous, 3.0 mL) was added and the solution was stirred at rt overnight. The reaction mixture was then diluted with DCM, washed with saturated aqueous sodium bicarbonate and dried over MgSO₄. The filtrate was evaporated onto silica gel. Purification by column chromatography (10 to 100% Jan. 9, 1990 ammonium hydroxide/MeOH/DCM: DCM) provided the title compound as an off-white solid, 0.107 g (88%). MS (ESI): 539.2 [M+H]⁺.

### Step D—Methyl 5-(5-{2-[(N,N-dimethylglycyl)amino]-4-pyridinyl}-1H-benzimidazol-1-yl)-3-({(1R)-1-[2-(trifluoromethyl)phenyl]ethyl}oxy)-2-thiophenecarboxylate

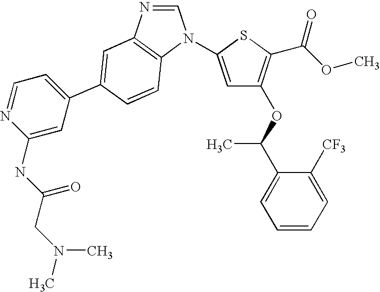

To a solution of methyl 5-[5-(2-amino-4-pyridinyl)-1H-benzimidazol-1-yl]-3-({(1R)-1-[2-(trifluoromethyl)phenyl]ethyl}oxy)-2-thiophenecarboxylate (0.043 g, 0.08 mmol), N,N-dimethyl glycine (0.013 g, 0.10 mmol) and diisopropylethylamine (0.052 mL, 0.24 mmol), in N,N-dimethylformamide (1.0 mL), 2-(7-aza-1H-benzotriazole-1-yl)-1,1,3,3-tetramethyluronium hexafluorophosphate (0.057 g, 0.12 mmol) was added and the solution was allowed to stir for 14 h at rt. The reaction mixture was then diluted with EtOAc and concentrated onto silica gel. Purification by column chromatography (20-100% EtOAc in hexanes, with 0.5% triethylamine) provided 0.030 g (60%) of the title compound as a foam. MS (ESI): 624.2 [M+H]⁺.

### Step E—5-(5-{2-[(N,N-dimethylglycyl)amino]-4-pyridinyl}-1H-benzimidazol-1-yl)-3-({(1R)-1-[2-(trifluoromethyl)phenyl]ethyl}oxy)-2-thiophenecarboxamide (title compound)

The title compound was prepared from methyl 5-(5-{2-[(N,N-dimethylglycyl)amino]-4-pyridinyl}-1H-benzimidazol-1-yl)-3-({(1R)-1-[2-(trifluoromethyl)phenyl]ethyl}oxy)-2-thiophenecarboxylate (30 mg, 0.05 mmol) by a procedure analogous to Example 1, Step D. The title compound was isolated in 41% yield as a white solid (12 mg). ¹H NMR (400 MHz, CDCl₃): δ 9.78 (brs, 1H), 8.58 (s, 1H), 8.36 (d, 1H, J=5.3 Hz), 8.13 (d, 1H, J=1.1 Hz), 7.99 (s, 1H), 7.76-7.62 (m, 4H), 7.51-7.46 (m, 2H), 7.33 (dd, 1H, J=5.2, 1.6 Hz), 7.22 (br s, 1H), 6.68 (s, 1H), 5.94 (br s, 1H), 5.87 (q, 1H, J=6.2 Hz), 3.17 (s, 2H), 2.43 (s, 6H), 1.82 (d, 3H, J=6.2 Hz); MS (ESI): 609.3 [M+H]⁺.

### EXAMPLE 4

### 5-{5-[2-(Glycylamino)-4-pyridinyl]-1H-benzimidazol-1-yl}-3-({(1R)-1-[2-(trifluoromethyl)phenyl]ethyl}oxy)-2-thiophenecarboxamide

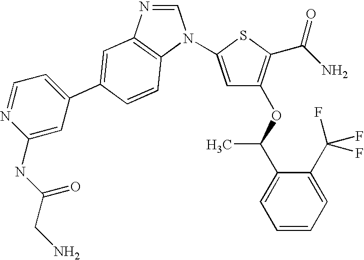

### Step A—5-[5-(2-Amino-4-pyridinyl)-1H-benzimidazol-1-yl]-3-({(1R)-1-[2-(trifluoromethyl)phenyl]ethyl}oxy)-2-thiophenecarboxamide

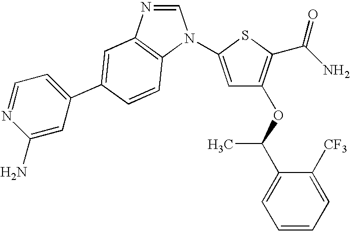

The title compound was prepared from methyl 5-[5-(2-amino-4-pyridinyl)-1H-benzimidazol-1-yl]-3-({(1R)-1-[2-(trifluoromethyl)phenyl]ethyl}oxy)-2-thiophenecarboxylate (0.107 g, 0.199 mmol) (prepared according to Example 3, Step C) and 8 mL of 7 N ammonia in MeOH heating in a sealed vessel at 85° C. for 10 h. The mixture was evaporated onto silica gel and purified by column chromatography (10 to 90% Jan. 9, 1990 ammonium hydroxide/MeOH/DCM: DCM) to give 0.085 g (82%) of desired product as a white solid. ¹H NMR (400 MHz, DMSO-d₆): δ 8.58 (s, 1H), 7.98 (s, 1H), 7.92 (m, 2H), 7.83 (brs, 1H), 7.76 (m, 2H), 7.57 (m, 3H), 7.14 (s, 1H), 7.12 (br s, 1H), 6.84 (d, 1H, J=5.3 Hz), 6.75 (s, 1H), 5.95 (m, 3H), 1.73 (d, 3H, J=6.0 Hz). HRMS C₂₆H₂₁N₅O₂F₃S: [M+H]⁺ calc'd. 524.1368, found 524.1367.

### Step B—1,1-Dimethylethyl {2-[(4-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]-2-oxoethyl}carbamate

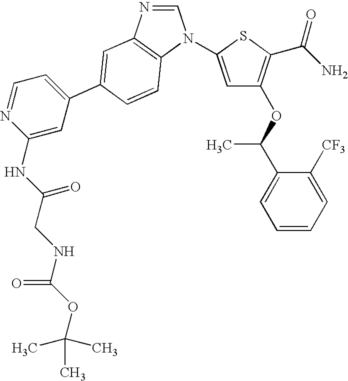

To a solution of 5-[5-(2-amino-4-pyridinyl)-1H-benzimidazol-1-yl]-3-({(1R)-1-[2-(trifluoromethyl)phenyl]ethyl}oxy)-2-thiophenecarboxamide (0.058 g, 0.011 mmol), N-{[(1,1-dimethylethyl)oxy]carbonyl}glycine (0.023 g, 0.13 mmol) and diisopropylethylamine (0.057 mL, 0.33 mmol), in DMF (1.0 mL), 2-(7-aza-1H-benzotriazole-1-yl)-1,1,3,3-tetramethyluronium hexafluorophosphate (0.063 g, 0.16 mmol) was added and the solution was allowed to stir for 14 h at rt. The reaction mixture was then diluted with EtOAc and concentrated onto silica gel. Purification by column chromatography ((10 to 90% Jan. 9, 1990 ammonium hydroxide/MeOH/DCM: DCM) provided 0.055 g (76%) of the title compound as a white solid. MS (ESI): 681.2 [M+H]⁺.

### Step C—5-{5-[2-(Glycylamino)-4-pyridinyl]-1H-benzimidazol-1-yl}-3-({(1R)-1-[2-(trifluoromethyl)phenyl]ethyl}oxy)-2-thiophenecarboxamide (title compound)

A solution of 1,1-dimethylethyl {2-[(4-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]-2-oxoethyl}carbamate (0.055 g, 0.08 mmol) and TFA (0.5 mL) was stirred in DCM (2 mL) at rt overnight. Excess solvents were removed in vacuo and the reaction mixture was concentrated onto silica gel. Purification by column chromatography (10-90% 90/9/1 DCM/MeOH/ammonium hydroxide:DCM) provided 0.026 g (55%) of the title compound as a white solid. ¹H NMR (400 MHz, CDCl₃): δ 9.90 (br s, 1H), 8.59 (s, 1H), 8.36 (d, 1H, J=5.3 Hz), 8.13 (s, 1H), 7.99 (s, 1H), 7.76-7.62 (m, 4H), 7.51-7.46 (m, 2H), 7.33 (d, 1H, J=5.1 Hz), 7.22 (br s, 1H), 6.68 (s, 1H), 5.92 (br s, 1H), 5.87 (q, 1H, J=6.3 Hz), 3.55 (s, 2H), 1.82 (d, 3H, J=6.0 Hz), 1.65 (br s, 2H); MS (AP): 581.1 [M+H]⁺.

### EXAMPLE 5

### 5-[5-(2-{[3-(Dimethylamino)propyl]amino}-4-pyridinyl)-1H-benzimidazol-1-yl]-3-([(1R)-1-[2-(trifluoromethyl)phenyl]ethyl]oxy)-2-thiophenecarboxamide

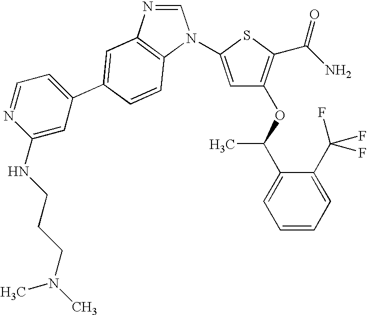

A solution of 5-[5-(2-fluoro-4-pyridinyl)-1H-benzimidazol-1-yl]-3-({(1R)-1-[2-(trifluoromethyl)phenyl]ethyl}oxy)-2-thiophenecarboxamide (0.205 g, 0.389 mmol) (prepared according to Example 1, Step E), N,N-dimethyl-1,3-propanediamine (1.0 mL) and i-PrOH (0.5 mL) was heated in a Personal Chemistry microwave at 180° C. for 42 min after which time the mixture was diluted with DCM and washed with water. The aqueous layer was extracted with DCM (x2), the organics were combined, dried over MgSO₄ and concentrated onto silica gel. Purification by column chromatography (10-100% 90/9/1 DCM/MeOH/ammonium hydroxide:DCM) provided 0.165 g (yellow solid) of product contaminated with N,N-dimethyl-1,3-propanediamine. The yellow solid was dissolved in 3 mL MeOH and added to 20 mL distilled water. Following removal of the MeOH co-solvent in vacuo, the desired product crystallized out of solution to give a white solid (110 mg, 46%) after filtration. ¹H NMR (400 MHz, CDCl₃): δ 8.11 (d, 1H, J=5.3 Hz), 8.02 (d, 1H, J=1.1 Hz), 7.95 (s, 1H), 7.72 (d, 1H, J=7.9 Hz), 7.69 (d, 1H, J=8.1 Hz), 7.62 (t, 1H, J=7.6 Hz), 7.56 (dd, 1H, J=8.4, 1.5 Hz), 7.47 (m, 2H), 7.18 (brs, 1H), 6.81 (dd, 1H, J=5.3, 1.3 Hz), 6.66 (s, 1H), 6.61 (s, 1H), 6.07 (br s, 1H), 5.85 (q, 1H, J=6.2 Hz), 3.42 (t, 2H, J=6.6 Hz), 2.48 (t, 2H, J=6.8 Hz), 2.30 (s, 6H), 1.87-1.81 (m, 2H), 1.79 (d, 3H, J=6.2 Hz); MS (AP): 609.3 [M+H]⁺.

### EXAMPLE 6

### 5-(5-{2-[(2-Aminoethyl)amino]-4-pyridinyl}-1H-benzimidazol-1-yl)-3-([(1R)-1-[2-(trifluoromethyl)phenyl]ethyl]oxy)-2-thiophenecarboxamide

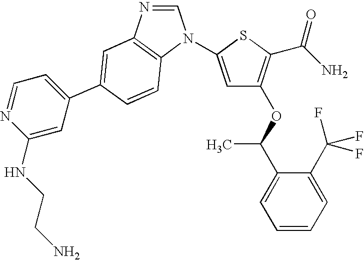

### Step A—1,1-Dimethylethyl {2-[(4-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]ethyl}carbamate

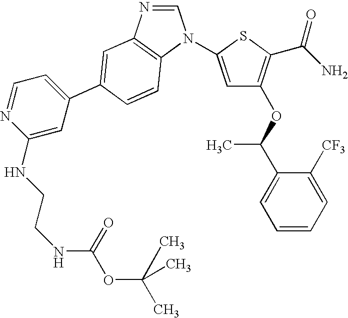

A solution of 5-[5-(2-fluoro-4-pyridinyl)-1H-benzimidazol-1-yl]-3-({(1R)-1-[2-(trifluoromethyl)phenyl]ethyl}oxy)-2-thiophenecarboxamide (0.100 g, 0.019 mmol) (prepared according to Example 1, Step E) and tert-butyl (2-aminoethyl)carbamate (0.5 mL) in EtOH (95%) was heated in a sealed tube at 115° C. for 24 h after which time the mixture was diluted with EtOAc and concentrated onto silica gel. Purification by column chromatography (30-100% EtOAc in hexanes, with 0.5% triethylamine) provided 0.088 g (70%) of the title compound as a foam. MS (ESI): 667.3 [M+H]⁺.

### Step B—5-(5-{2-[(2-Aminoethyl)amino]-4-pyridinyl}-1H-benzimidazol-1-yl)-3-({(1R)-1-[2-(trifluoromethyl)phenyl]ethyl}oxy)-2-thiophenecarboxamide (title compound)

The title compound was prepared from 1,1-dimethylethyl {2-[(4-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]ethyl}carbamate (88 mg, 0.13 mmol) by a procedure analogous to Example 2, Step B. The title compound was isolated in 40% yield as a light yellow solid (30 mg). ¹H NMR (400 MHz, CDCl₃): δ 8.13 (d, 1H, J=5.3 Hz), 8.02 (s, 1H), 7.99 (s, 1H), 7.72 (d, 1H, J=8.0 Hz), 7.69 (d, 1H, J=8.2 Hz), 7.62 (t, 1H, J=7.5 Hz), 7.55 (d, 1H, J=9.5 Hz), 7.48-7.44 (m, 2H), 7.20 (br s, 1H), 6.83 (d, 1H, J=5.3 Hz), 6.66 (s, 1H), 6.64 (s, 1H), 6.09 (br s, 1H), 5.85 (q, 1H, J=6.2 Hz), 4.95 (t, 1H, J=5.6 Hz), 3.43 (q, 2H, J=5.7 Hz), 2.98 (t, 2H, J=5.6 Hz), 1.79 (d, 3H, J=6.2 Hz), 1.45 (br s, 2H); MS (AP): 567.1 [M+H]⁺.

### EXAMPLE 7

### 5-(5-{2-[[2-(Dimethylamino)ethyl](methyl)amino]-4-pyridinyl}-1H-benzimidazol-1-yl)-3-({(1R)-1-[2-(trifluoromethyl)phenyl]ethyl}oxy)-2-thiophenecarboxamide

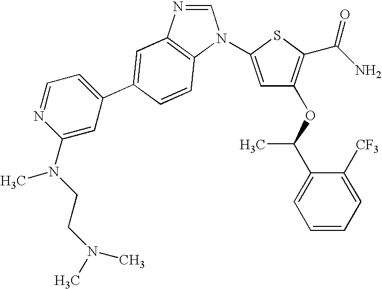

A solution of 5-[5-(2-fluoro-4-pyridinyl)-1H-benzimidazol-1-yl]-3-({(1R)-1-[2-(trifluoromethyl)phenyl]ethyl}oxy)-2-thiophenecarboxamide (0.100 g, 0.19 mmol) (prepared according to Example 1, Step E), N,N,N′-trimethyl-1,2-ethanediamine (0.5 mL) and EtOH (95%, 0.5 mL) was heated in a Personal Chemistry microwave at 180° C. for 35 min after which time the mixture was diluted with EtOAc, washed with water, dried over MgSO₄ and concentrated onto silica gel. Purification by column chromatography (10-90% 90/9/1 DCM/MeOH/ammonium hydroxide:DCM) provided 0.073 g (63%) of the title compound as a tan solid. ¹H NMR (400 MHz, CDCl₃): δ 8.21 (d, 1H, J=5.3 Hz), 8.05 (s, 1H), 7.97 (s, 1H), 7.74 (d, 1H, J=8.1 Hz), 7.71 (d, 1H, J=7.9 Hz), 7.64 (t, 1H, J=7.6 Hz), 7.58 (dd, 1H, J=8.4, 1.5 Hz), 7.52-7.45 (m, 2H), 7.21 (br s, 1H), 6.81 (d, 1H, J=5.9 Hz), 6.71 (s, 1H), 6.68 (s, 1H), 5.87 (q, 1H, J=6.3 Hz), 5.82 (br s, 1H), 3.78 (t, 2H, J=6.0 Hz), 3.14 (s, 3H), 2.61 (br s, 2H), 2.36 (br s, 6H), 1.82 (d, 3H, J=6.2 Hz); MS (ESI): 609.3 [M+H]⁺.

### EXAMPLE 8

### 5-(5-{2-[[3-(Dimethylamino)propyl](methyl)amino]-4-pyridinyl}-1H-benzimidazol-1-yl)-3-({(1R)-1-[2-(trifluoromethyl)phenyl]ethyl}oxy)-2-thiophenecarboxamide

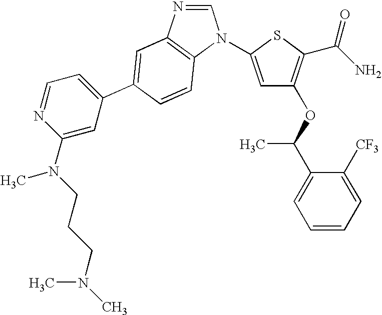

The title compound was prepared from 5-[5-(2-fluoro-4-pyridinyl)-1H-benzimidazol-1-yl]-3-({(1R)-1-[2-(trifluoromethyl)phenyl]ethyl}oxy)-2-thiophenecarboxamide (100 mg, 0.19 mmol) and N,N,N′N-trimethyl-1,3-propanediamine (0.5 mL, 3.8 mmol) by a procedure analogous to Example 7. The title compound was isolated in 86% yield as a tan solid (101 mg). ¹H NMR (400 MHz, CDCl₃): δ 8.20 (d, 1H, J=5.3 Hz), 8.06 (d, 1H, J=1.1 Hz), 7.97 (s, 1H), 7.73 (d, 1H, J=8.4 Hz), 7.71 (d, 1H, J=8.0 Hz), 7.64 (t, 1H, J=7.6 Hz), 7.59 (dd, 1H, J=8.5, 1.6 Hz), 7.53-7.45 (m, 2H), 7.19 (brs, 1H), 6.80 (dd, 1H, J=5.9, 1.1 Hz), 6.74 (s, 1H), 6.68 (s, 1H), 5.99 (br s, 1H), 5.87 (q, 1H, J=6.2 Hz), 3.65 (t, 2H, J=7.1 Hz), 3.12 (s, 3H), 2.40 (t, 2H, J=7.3 Hz), 2.29 (s, 6H), 1.88-1.82 (m, 2H), 1.81 (d, 3H, J=6.4 Hz); MS (ESI): 623.3 [M+H]⁺.

### EXAMPLE 9

### 5-[5-(2-{[3-(4-Methyl-1-piperazinyl)propyl]amino}-4-pyridinyl)-1H-benzimidazol-1-yl]-3-({(1R)-1-[2-(trifluoromethyl)phenyl]ethyl}oxy)-2-thiophenecarboxamide

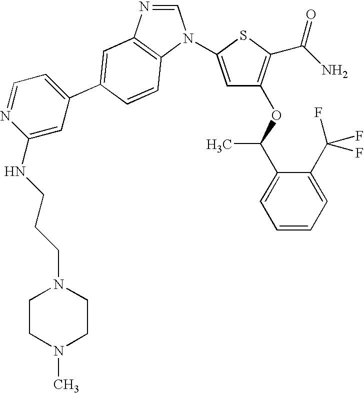

The title compound was prepared from 5-[5-(2-fluoro-4-pyridinyl)-1H-benzimidazol-1-yl]-3-({(1R)-1-[2-(trifluoromethyl)phenyl]ethyl}oxy)-2-thiophenecarboxamide (166 mg, 0.315 mmol) and [3-(4-methyl-1-piperazinyl)propyl]amine (1.0 mL, 5.9 mmol) by a procedure analogous to Example 7. The title compound was isolated in 66% yield as a yellow solid (137 mg). ¹H NMR (400 MHz, CDCl₃): δ 8.14 (d, 1H, J=5.5 Hz), 8.04 (s, 1H), 7.97 (s, 1H), 7.73 (d, 1H, J=8.0 Hz), 7.71 (d, 1H, J=8.6 Hz), 7.64 (t, 1H, J=7.7 Hz), 7.57 (dd, 1H, J=8.6, 1.5 Hz), 7.52-7.45 (m, 2H), 7.20 (br s, 1H), 6.82 (dd, 1H, J=5.3, 1.3 Hz), 6.67 (s, 1H), 6.60 (s, 1H), 5.87 (q, 1H, J=6.2 Hz), 5.79 (br s, 1H), 5.40 (t, 1H, J=5.1 Hz), 3.43 (q, 2H, J=6.0 Hz), 2.70-2.32 (br s, 8H), 2.52 (t, 2H, J=6.8 Hz), 2.29 (s, 3H), 1.88-1.83 (m, 2H), 1.81 (d, 3H, J=6.4 Hz); MS (ESI): 664.3 [M+H]⁺.

### EXAMPLE 10

### 3-{[(1R)-1-(2-chlorophenyl)ethyl]oxy}-5-[5-(2-{[2-(dimethylamino)ethyl]amino}-4-pyridinyl)-1H-benzimidazol-1-yl]-2-thiophenecarboxamide

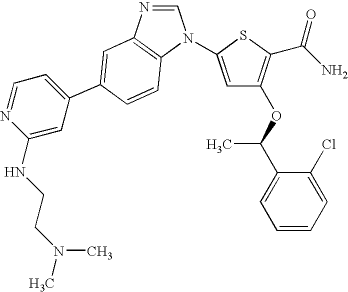

### Step A—Methyl 5-[(4-bromo-2-nitrophenyl)amino]-3-{[(1R)-1-(2-chlorophenyl)ethyl]oxy}thiophene-2-carboxylate

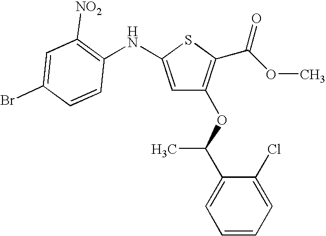

The title compound was prepared from methyl 5-amino-3-{[(1R)-1-(2-chlorophenyl)ethyl]oxy}-2-thiophenecarboxylate (8.58 g, 30.5 mmol) (prepared according to Intermediate Example 2) by a procedure analogous to Example 1, Step A. The title compound was obtained as a red semi-solid (9.7 g). MS (ESI): 534.0 [M+Na]⁺.

### Step B—Methyl 5-[(2-amino-4-bromophenyl)amino]-3-{[(1R)-1-(2-chlorophenyl)ethyl]oxy}thiophene-2-carboxylate

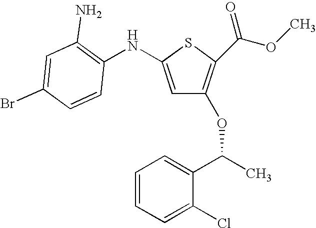

The title compound was prepared from methyl 5-[(4-bromo-2-nitrophenyl)amino]-3-{[(1R)-1-(2-chlorophenyl)ethyl]oxy}thiophene-2-carboxylate (9.7 g, 19.0 mmol) by a procedure analogous to Example 1, Step B. The title compound was obtained as a yellow solid (9.1 g). MS (ESI): 482.0 [M+H]⁺.

### Step C—Methyl 5-(5-bromo-1H-benzimidazol-1-yl)-3-{[(1R)-1-(2-chlorophenyl)ethyl]oxy}thiophene-2-carboxylate

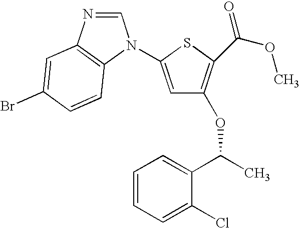

The title compound was prepared from methyl 5-[(2-amino-4-bromophenyl)amino]-3-{[(1R)-1-(2-chlorophenyl)ethyl]oxy}thiophene-2-carboxylate (9.1 g, 18.9 mmol) by a procedure analogous to Example 1, Step C. The title compound was obtained as a light yellow solid (8.3 g). ¹H NMR (300 MHz, CDCl₃): δ 8.02 (s, 1H), 7.70 (dd, 1H, J=7.6, 1.8 Hz), 7.51-7.28 (m, 6H), 6.74 (s, 1H), 5.86 (q, 1H, J=6.3 Hz), 3.97 (s, 3H), 1.79 (d, 3H, J=6.5 Hz); MS (APCI): 492.79 [M+H]⁺.

### Step D—5-(5-Bromo-1H-benzimidazol-1-yl)-3-{[(1R)-1-(2-chlorophenyl)ethyl]-oxy}thiophene-2-carboxamide

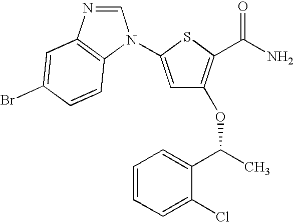

The title compound was prepared from methyl 5-(5-bromo-1H-benzimidazol-1-yl)-3-{[(1R)-1-(2-chlorophenyl)ethyl]oxy}thiophene-2-carboxylate (5.8 g, 11.8 mmol) by a procedure analogous to Example 1, Step D. The title compound was obtained as a white solid (5.1 g). ¹H NMR (400 MHz, CDCl₃): δ 8.00 (s, 1H), 7.97 (d, J=1.3 Hz, 1H), 7.46-7.41 (m, 3H), 7.35-7.26 (m, 3H), 7.17 (br s, 1H), 6.60 (s, 1H), 5.85 (q, J=6.4 Hz, 1H), 5.75 (br s, 1H), 1.76 (d, J=6.4 Hz, 3H). MS (APCI): 478.0 [M+H]⁺.

### Step E—3-{[(1R)-1-(2-chlorophenyl)ethyl]oxy}-5-[5-(2-fluoropyridin-4-yl)-1H-benzimidazol-1-yl]thiophene-2-carboxamide

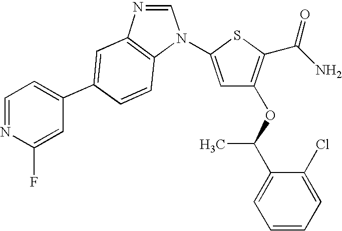

2-Fluoropyridine-4-boronic acid (0.440 g, 3.15 mmol), 5-(5-bromo-1H-benzimidazol-1-yl)-3-{[(1R)-1-(2-chlorophenyl)ethyl]oxy}thiophene-2-carboxamide (1.0 g, 2.10 mmol), 1,1′ bisdiphenylphosphinoferrocene dichloropalladium (II) (0.171 g, 0.21 mmol), and sodium carbonate (1N in water, 7.4 mL) were combined in N,N-dimethylacetamide (25 mL). The reaction mixture was stirred under nitrogen while heating at 80° C. for 2 h after which time the mixture was cooled and partitioned between DCM and water. The aqueous layer was extracted (×5) with DCM. The combined organics were dried over MgSO₄, concentrated onto silica gel and purified by column chromatography (10-100% EtOAc in hexanes) to afford 0.735 g (71%) as a tan solid. ¹H NMR (400 MHz, CDCl₃): δ 8.33 (d, 1H, J=5.3 Hz), 8.11 (d, 2H, J=16.0 Hz), 7.50 (m, 7H), 7.26 (s, 1H), 7.23 (br s, 1H), 6.71 (s, 1H), 5.94 (q, 1H, J=6.3 Hz), 5.82 (br s, 1H), 1.84 (d, 3H, J=6.5 Hz); MS (ESI): 493.1 [M+H]⁺.

### Step F—3-{[(1R)-1-(2-chlorophenyl)ethyl]oxy}-5-[5-(2-{[2-(dimethylamino)ethyl]amino}-4-pyridinyl)-1H-benzimidazol-1-yl]-2-thiophenecarboxamide (title compound)

The title compound was prepared from 3-{[(1R)-1-(2-chlorophenyl)ethyl]oxy}-5-[5-(2-fluoropyridin-4-yl)-1H-benzimidazol-1-yl]thiophene-2-carboxamide (200 mg, 0.41 mmol) and N,N-dimethyl-1,2-ethanediamine (0.60 mL, 5.5 mmol) by a procedure analogous to Example 7. The title compound was isolated in 63% yield as a light brown solid (140 mg). ¹H NMR (400 MHz, CDCl₃): δ 8.15 (d, 1H, J=5.5 Hz), 8.05 (d, 1H, J=1.3 Hz), 8.01 (s, 1H), 7.58 (dd, 1H, J=8.5, 1.6 Hz), 7.50-7.43 (m, 3H), 7.37-7.23 (m, 2H), 7.21 (br s, 1H), 6.85 (dd, 1H, J=5.4, 1.4 Hz), 6.68 (s, 1H), 6.65 (s, 1H), 5.89 (q, 1H, J=6.3 Hz), 5.77 (br s, 1H), 5.30 (br s, 1H), 3.49 (q, 2H, J=5.0 Hz), 2.67 (br s, 2H), 2.35 (s, 6H), 1.79 (d, 3H, J=6.4 Hz); MS (ESI): 561.3 [M+H]⁺.

### EXAMPLE 11

### 3-{[(1R)-1-(2-chlorophenyl)ethyl]oxy}-5-[5-(2-{[3-(4-methyl-1-piperazinyl)propyl]amino}-4-pyridinyl)-1H-benzimidazol-1-yl]-2-thiophenecarboxamide

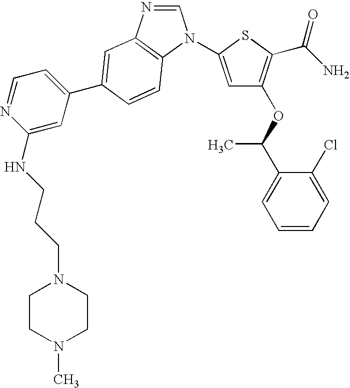

The title compound was prepared from 3-{[(1R)-1-(2-chlorophenyl)ethyl]oxy}-5-[5-(2-fluoropyridin-4-yl)-1H-benzimidazol-1-yl]thiophene-2-carboxamide (200 mg, 0.41 mmol) (prepared according to Example 10) and [3-(4-methyl-1-piperazinyl)propyl]amine (1.0 mL, 5.9 mmol) by a procedure analogous to Example 7. The title compound was isolated in 65% yield as a yellow solid (167 mg). ¹H NMR (400 MHz, CDCl₃): δ 8.13 (d, 1H, J=5.3 Hz), 8.03 (s, 1H), 7.99 (s, 1H), 7.56 (d, 1H, J=8.6 Hz), 7.49-7.41 (m, 3H), 7.36-7.27 (m, 2H), 7.19 (br s, 1H), 6.81 (d, 1H, J=5.3 Hz), 6.64 (s, 1H), 6.60 (s, 1H), 5.87 (q, 1H, J=6.2 Hz), 5.81 (br s, 1H), 5.40 (s, 1H), 3.43 (q, 2H, J=5.8 Hz), 2.60-2.40 (br s, 10H), 2.32 (s, 3H), 1.88-1.80 (m, 2H), 1.77 (d, 3H, J=6.2 Hz); MS (ESI): 630.3 [M+H]⁺.

### EXAMPLE 12

### 3-{[(1R)-1-(2-chlorophenyl)ethyl]oxy}-5-[5-(2-{[2-(4-morpholinyl)ethyl]amino}-4-pyridinyl)-1H-benzimidazol-1-yl]-2-thiophenecarboxamide

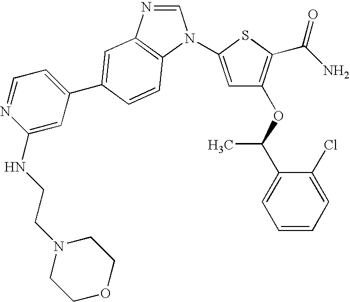

The title compound was prepared from 3-{[(1R)-1-(2-chlorophenyl)ethyl]oxy}-5-[5-(2-fluoropyridin-4-yl)-1H-benzimidazol-1-yl]thiophene-2-carboxamide (90 mg, 0.18 mmol) (prepared according to Example 10, Step E) and [2-(4-morpholinyl)ethyl]amine (0.5 mL, 3.8 mmol) by a procedure analogous to Example 7. The title compound was isolated in 67% yield as a beige solid (74 mg). ¹H NMR (400 MHz, CDCl₃): δ 8.14 (d, 1H, J=5.3 Hz), 8.04 (s, 1H), 7.99 (s, 1H), 7.57 (d, 1H, J=8.6 Hz), 7.49-7.45 (m, 2H), 7.43 (d, 1H, J=7.7 Hz), 7.36-7.27 (m, 2H), 7.19 (br s, 1H), 6.85 (d, 1H, J=5.1 Hz), 6.64 (s, 2H), 5.87 (q, 1H, J=6.4 Hz), 5.83 (br s, 1H), 5.22 (s, 1H), 3.73 (t, 4H, J=4.2 Hz), 3.49-3.41 (m, 2H), 2.66 (t, 2H, J=5.8 Hz), 2.50 (m, 4H), 1.77 (d, 3H, J=6.2 Hz); MS (ESI): 603.1 [M+H]⁺.

### EXAMPLE 13

### 3-{[(1R)-1-(2-chlorophenyl)ethyl]oxy}-5-[5-(2-{[(2R)-2,3-dihydroxypropyl]amino}-4-pyridinyl)-1H-benzimidazol-1-yl]-2-thiophenecarboxamide

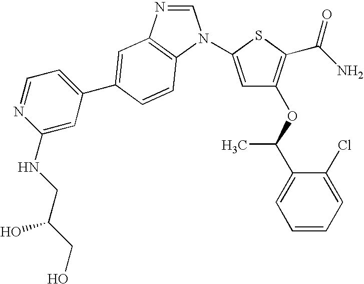

The title compound was prepared from 3-{[(1R)-1-(2-chlorophenyl)ethyl]oxy}-5-[5-(2-fluoropyridin-4-yl)-1H-benzimidazol-1-yl]thiophene-2-carboxamide (160 mg, 0.34 mmol) (prepared according to Example 10, Step E) and (2R)-3-amino-1,2-propanediol (0.8 mL, 8.8 mmol) by a procedure analogous to Example 7. The title compound was isolated in 41% yield as a tan solid (78 mg). ¹H NMR (400 MHz, CDCl₃): δ 8.08 (d, 1H, J=5.5 Hz), 8.01 (s, 1H), 7.99 (s, 1H), 7.56 (d, 1H, J=8.6 Hz), 7.49-7.45 (m, 2H), 7.41 (t, 1H, J=7.8 Hz), 7.36-7.26 (m, 2H), 7.18 (m, 1H), 6.88 (d, 1H, J=5.5 Hz), 6.68 (s, 1H), 6.64 (s, 1H), 5.87 (q, 1H, J=6.4 Hz), 5.74 (br s, 1H), 4.81 (t, 1H, J=5.8 Hz), 3.84-3.79 (m, 1H), 3.71-3.65 (m, 1H), 3.65-3.58 (m, 4H), 2.70-2.58 (m, 1H), 1.77 (d, 3H, J=6.4 Hz); MS (ESI): 564.3 [M+H]⁺.

### EXAMPLE 14

### 3-{[(1R)-1-(2-chlorophenyl)ethyl]oxy}-5-(5-{2-[(2-hydroxyethyl)amino]-4-pyridinyl}-1H-benzimidazol-1-yl)-2-thiophenecarboxamide

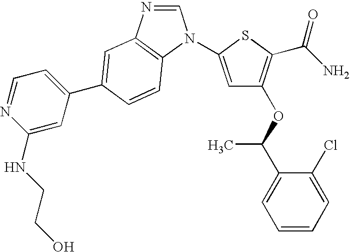

The title compound was prepared from 3-{[(1R)-1-(2-chlorophenyl)ethyl]oxy}-5-[5-(2-fluoropyridin-4-yl)-1H-benzimidazol-1-yl]thiophene-2-carboxamide (270 mg, 0.55 mmol) (prepared according to Example 10, Step E) and ethanolamine (1.0 mL, 16.6 mmol) by a procedure analogous to Example 5. The title compound was isolated in 54% yield as a tan solid (158 mg). ¹H NMR (400 MHz, CDCl₃): δ 8.09-8.05 (m, 1H), 8.02 (s, 1H), 7.99 (s, 1H), 7.54 (d, 1H, J=8.6 Hz), 7.49-7.45 (m, 2H), 7.42 (d, 1H, J=7.6 Hz), 7.36-7.27 (m, 2H), 7.19 (br s, 1H), 6.90-6.86 (m, 1H), 6.71 (s, 1H), 6.64 (s, 1H), 5.87 (q, 1H, J=6.4 Hz), 5.78 (br s, 1H), 5.20 (br s, 1H), 3.85 (t, 2H, J=4.5 Hz), 3.58 (q, 2H, J=4.0 Hz), 1.77 (d, 3H, J=6.2 Hz); MS (ESI): 534.2 [M+H]⁺.

### EXAMPLE 15

### 2-({4-[1-(5-(Aminocarbonyl)-4-{[(1R)-1-(2-chlorophenyl)ethyl]oxy}-2-thienyl)-1H-benzimidazol-5-yl]-2-pyridinyl}amino)ethyl L-valinate

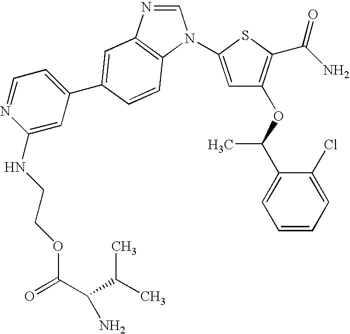

### Step A—2-({4-[1-(5-(Aminocarbonyl)-4-{[(1R)-1-(2-chlorophenyl)ethyl]oxy}-2-thienyl)-1H-benzimidazol-5-yl]-2-pyridinyl}amino)ethyl N-{[(1,1-dimethylethyl)oxy]carbonyl}-L-valinate

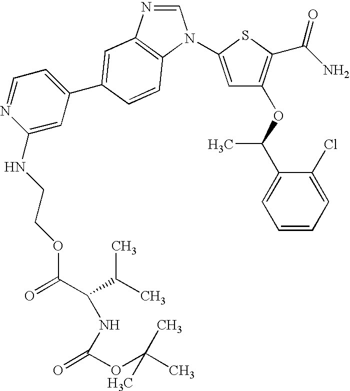

To a solution of 3-{[(1R)-1-(2-chlorophenyl)ethyl]oxy}-5-(5-{2-[(2-hydroxyethyl)amino]-4-pyridinyl}-1H-benzimidazol-1-yl)-2-thiophenecarboxamide (0.093 g, 0.17 mmol) (prepared according to Example 10), N-{[(1,1-dimethylethyl)oxy]carbonyl}-L-valine (0.057 g, 0.26 mmol) and diisopropylethylamine (0.05 mL, 0.24 mmol), in DMF (1.0 mL), 2-(7-aza-1H-benzotriazole-1-yl)-1,1,3,3-tetramethyluronium hexafluorophosphate (0.080 g, 0.21 mmol) was added and the solution was allowed to stir for 24 h at rt. The reaction mixture was then diluted with DCM, washed with water and extracted twice with DCM. The combined organics were dried over MgSO₄ and concentrated onto silica gel. 10 to 50% Jan. 9, 1990 ammonium hydroxide/MeOH/DCM: DCM) to give the desired product as a foam, 0.055 g (43%). MS (ESI): 733.4 [M+H]⁺.

### Step B—2-({4-[1-(5-(Aminocarbonyl)-4-{[(1R)-1-(2-chlorophenyl)ethyl]oxy}-2-thienyl)-1H-benzimidazol-5-yl]-2-pyridinyl}amino)ethyl L-valinate (title compound)

The title compound was prepared from 2-({4-[1-(5-(aminocarbonyl)-4-{[(1R)-1-(2-chlorophenyl)ethyl]oxy}-2-thienyl)-1H-benzimidazol-5-yl]-2-pyridinyl}amino)ethyl N-{[(1,1-dimethylethyl)oxy]carbonyl}-L-valinate (55 mg, 0.075 mmol) by a procedure analogous to Example 2, Step B. The title compound was isolated in 100% yield as a beige solid (48 mg). ¹H NMR (400 MHz, CDCl₃): δ 8.09 (d, 1H, J=5.5 Hz), 7.99 (s, 1H), 7.97 (s, 1H), 7.52-7.40 (m, 4H), 7.36-7.26 (m, 2H), 7.20 (br s, 1H), 6.82 (d, 1H, J=4.8 Hz), 6.68 (s, 1H), 6.63 (s, 1H), 6.18 (br s, 1H), 5.87 (q, 1H, J=6.3 Hz), 5.35 (br s, 1H), 4.45-4.37 (m, 1H), 4.36-4.30 (m, 1H), 3.72-3.63 (m, 2H), 3.34 (d, 1H, J=5.0 Hz), 2.30 (br s, 2H), 2.07-1.98 (m, 1H), 1.77 (d, 3H, J=6.2 Hz), 0.95 (d, 3H, J=6.8 Hz), 0.88 (d, 3H, J=7.0 Hz); MS (ESI): 633.3 [M+H]⁺.

### EXAMPLE 16

### 5-[5-(2-{[2-(Dimethylamino)ethyl]amino}pyrimidin-4-yl)-1H-benzimidazol-1-yl]-3-({(1R)-1-[2-(trifluoromethyl)phenyl]ethyl}oxy)thiophene-2-carboxamide

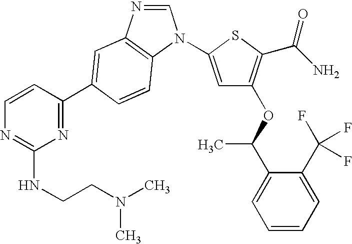

### Step A—5-[5-(4,4,5,5-Tetramethyl-1,3,2-dioxaborolan-2-yl)-1H-benzimidazol-1-yl]-3-({(1R)-1-[2-(trifluoromethyl)phenyl]ethyl}oxy)thiophene-2-carboxamide

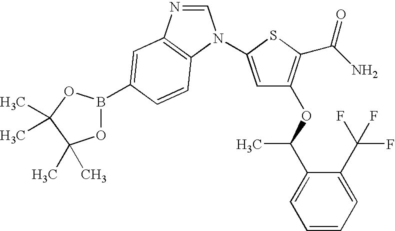

5-(5-Bromo-1H-benzimidazol-1-yl)-3-({(1R)-1-[2-(trifluoromethyl)phenyl]ethyl}oxy)thiophene-2-carboxamide (0.114 g, 0.223 mmol) (prepared according to Example 1, Step D), bis(pinacolato)diboron (0.068 g, 0.268 mmol), potassium acetate (0.055 g, 0.669 mmol), and dichlorobis(triphenylphosphine)palladium(II) (0.016 g, 0.022 mmol) were combined in DMF (1.5 mL). The reaction mixture was heated in a Personal Chemistry microwave at 150° C. for 20 min after which time the mixture was diluted with EtOAc and concentrated onto silica gel. Purification by column chromatography (10-100% EtOAc:hexanes) provided 0.062 g (50%) of the title compound as an off-white solid. MS (ESI): 558.2 [M+H]⁺. In a separate experiment, the title compound was also prepared from 1-[5-(aminocarbonyl)-4-({(1R)-1-[2-(trifluoromethyl)phenyl]ethyl}oxy)-2-thienyl]-1H-benzimidazol-5-yl trifluoromethanesulfonate instead of 5-(5-bromo-1H-benzimidazol-1-yl)-3-({(1R)-1-[2-(trifluoromethyl)phenyl]ethyl}oxy)thiophene-2-carboxamide following a procedure analogous to that described above (yield: 193 mg, 90%).

### Step B—5-[5-(2-Chloropyrimidin-4-yl)-1H-benzimidazol-1-yl]-3-({(1R)-1-[2-(trifluoromethyl)phenyl]ethyl}oxy)thiophene-2-carboxamide

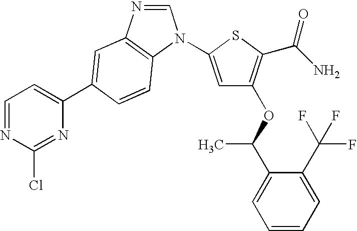

5-[5-(4,4,5,5-Tetramethyl-1,3,2-dioxaborolan-2-yl)-1H-benzimidazol-1-yl]-3-({(1R)-1-[2-(trifluoromethyl)phenyl]ethyl}oxy)thiophene-2-carboxamide (0.193 g, 0.350 mmol) (prepared using a prodecure analogous to Example 16, Step A, from 1-[5-(aminocarbonyl)-4-({(1R)-1-[2-(trifluoromethyl)phenyl]ethyl}oxy)-2-thienyl]-1H-benzimidazol-5-yl trifluoromethanesulfonate) and 2,4-dichloropyrimidine (0.089 g, 0.600 mmol) were dissolved in DMA (2.0 mL). Sodium carbonate (1 N in water, 0.7 mL) was added followed by 1,1′-bisdiphenylphosphinoferrocene dichloropalladium (II) (0.029 g, 0.035 mmol). The reaction mixture was stirred under nitrogen while heating at 80° C. for 15 h. The mixture was then cooled and partitioned between EtOAc and water.

The aqueous layer was extracted with EtOAc. The combined organics were dried over MgSO₄, concentrated onto silica gel and purified by column chromatography (10-100% EtOAc:hexanes) to afford 0.100 g (53%) of the title compound as a white solid. MS (ESI): 544.1 [M+H]⁺.

### Step C—5-[5-(2-{[2-(Dimethylamino)ethyl]amino}pyrimidin-4-yl)-1H-benzimidazol-1-yl]-3-({(1R)-1-[2-(trifluoromethyl)phenyl]ethyl}oxy)thiophene-2-carboxamide (title compound)

5-[5-(2-Chloropyrimidin-4-yl)-1H-benzimidazol-1-yl]-3-({(1R)-1-[2-(trifluoromethyl)phenyl]ethyl}oxy)thiophene-2-carboxamide (0.064 g, 0.116 mmol) and N,N-dimethyl-ethylenediamine (0.5 mL) were combined in 95% ethanol (1.5 mL). The reaction mixture was heated in a Personal Chemistry microwave at 180° C. for 15 min after which time the mixture was diluted with EtOAc and washed with water. The aqueous layer was extracted twice with EtOAc. The organics were combined, dried over MgSO₄, filtered and concentrated onto silica gel. Purification by column chromatography (10-100% ammonium hydroxide:MeOH:DCM 1:9:90 in DCM) provided 0.050 g (72%) as an off-white solid. ¹H NMR (400 MHz, CDCl₃): [symbol] 8.47 (s, 1H), 8.33 (d, 1H, J=5.1 Hz), 8.08 (d, 1H, J=8.6 Hz), 7.96 (s, 1H), 7.71 (dd, 2H, J=7.9, 14.1 Hz), 7.63 (t, 1H, J=7.5 Hz), 7.46 (m, 2H), 7.19 (br s, 1H), 7.02 (d, 1H, J=5.1 Hz), 6.66 (s, 1H), 5.85 (m, 2H), 5.73 (br s, 1H), 3.64 (d, 2H, J=4.2 Hz), 2.67 (m, 2H), 2.36 (s, 6H), 1.80 (d, 3H, J=6.2 Hz); MS (APCI): 596.1 [M+H]⁺.

### EXAMPLE 17

### 3-{[(1R)-1-(2-chlorophenyl)ethyl]oxy}-5-[5-(2-{[3-(4-methyl-1-piperazinyl)propyl]amino}-4-pyrimidinyl)-1H-benzimidazol-1-yl]-2-thiophenecarboxamide

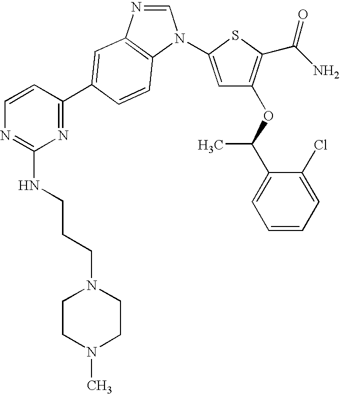

### Step A—3-{[(1R)-1-(2-chlorophenyl)ethyl]oxy}-5-[5-(4,4,5,5-tetramethyl-1,3,2-dioxaborolan-2-yl)-1H-benzimidazol-1-yl]thiophene-2-carboxamide

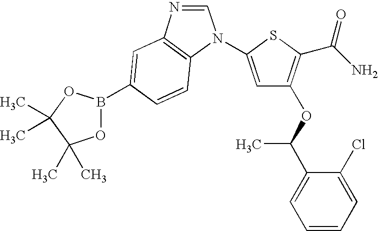

5-(5-bromo-1H-benzimidazol-1-yl)-3-{[(1R)-1-(2-chlorophenyl)ethyl]oxy}thiophene-2-carboxamide (0.521 g, 1.09 mmol) (prepared according to Example 10, Step D), bis(pinacolato)diboron (0.330 g, 1.30 mmol), potassium acetate (0.270 g, 3.30 mmol), and dichlorobis(triphenylphosphine)palladium(II) (0.077 g, 0.110 mmol) were combined in DMF (7.0 mL). The reaction mixture was divided equally between two 5 mL microwave vessels and both heated in a Personal Chemistry microwave at 150° C. for 20 min after which time the mixtures were diluted with EtOAc, combined, and concentrated onto silica gel. Purification by column chromatography (10-100% EtOAc:hexanes) provided 0.062 g (50%) of the title compound as an off-white solid. MS (ESI): 524.2 [M+H]⁺.

### Step B—3-{[(1R)-1-(2-chlorophenyl)ethyl]oxy}-5-[5-(2-chloropyrimidin-4-yl)-1H-benzimidazol-1-yl]thiophene-2-carboxamide

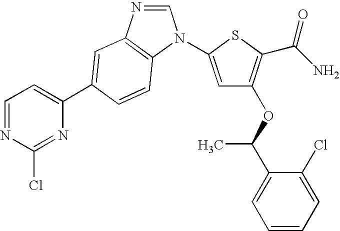

The title compound was prepared from 3-{[(1R)-1-(2-chlorophenyl)ethyl]oxy}-5-[5-(4,4,5,5-tetramethyl-1,3,2-dioxaborolan-2-yl)-1H-benzimidazol-1-yl]thiophene-2-carboxamide (50 mg, 0.95 mmol) by a procedure analogous to Example 16, Step B. The title compound was obtained as a white solid (485 mg). MS (ESI): 510.1 [M+H]⁺.

### Step C—3-{[(1R)-1-(2-chlorophenyl)ethyl]oxy}-5-[5-(2-{[3-(4-methyl-1-piperazinyl)propyl]amino}-4-pyrimidinyl)-1H-benzimidazol-1-yl]-2-thiophenecarboxamide (title compound)

The title compound was prepared from 3-{[(1R)-1-(2-chlorophenyl)ethyl]oxy}-5-[5-(2-chloropyrimidin-4-yl)-1H-benzimidazol-1-yl]thiophene-2-carboxamide (104 mg, 0.204 mmol) and [3-(4-methyl-1-piperazinyl)propyl]amine (0.5 mL, 3.0 mmol) by a procedure analogous to Example 16, Step C. The title compound was isolated as a yellow solid (64 mg, 48% yield). ¹H NMR (400 MHz, CDCl₃): δ 8.50 (s, 1H), 8.32 (d, 1H, J=5.1 Hz), 8.06 (d, 1H, J=8.6 Hz), 7.99 (s, 1H), 7.51-7.41 (m, 3H), 7.36-7.27 (m, 2H), 7.19 (br s, 1H), 7.01 (d, 1H, J=5.1 Hz), 6.64 (s, 1H), 5.87 (q, 1H, J=6.3 Hz), 5.77 (br s, 1H), 3.59 (m, 2H), 2.54 (m, 10H), 2.35 (br s, 3H), 1.84 (m, 2H), 1.77 (d, 3H, J=6.2 Hz); MS (AP): 631.1 [M+H]⁺.

### EXAMPLE 18

### 3-{[(1R)-1-(2-Chlorophenyl)ethyl]oxy}-5-[5-(2-{[3-(dimethylamino)propyl]amino}-5-pyrimidinyl)-1H-benzimidazol-1-yl]-2-thiophenecarboxamide

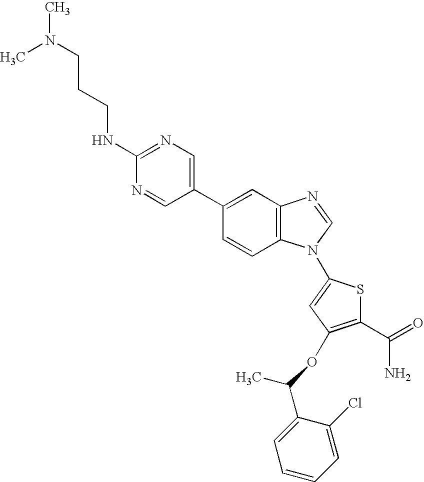

### Step A—3-{[(1R)-1-(2-Chlorophenyl)ethyl]oxy}-5-[5-(2-chloro-5-pyrimidinyl)-1H-benzimidazol-1-yl]-2-thiophenecarboxamide

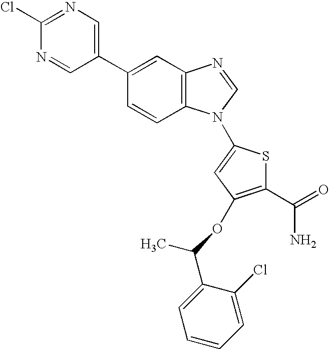

3-{[(1R)-1-(2-Chlorophenyl)ethyl]oxy}-5-[5-(4,4,5,5-tetramethyl-1,3,2-dioxaborolan-2-yl)-1H-benzimidazol-1-yl]thiophene-2-carboxamide (530 mg; 1.0 mmol) (prepare according to Example 17, Step A) and 5-bromo-2-chloropyrimidine (353 mg; 1.8 mmol) were dissolved in DMA (5 mL). The mixture was treated with 1 N aqueous sodium carbonate (2 mL) and 1,1-bisdiphenylphosphinoferracenedichloropalladium (II) (82 mg; 0.10 mmol). The mixture was heated to 80° C. for 1 h. The reaction was cooled to rt and partitioned between water and EtOAc. The layers were separated and the water layer was extracted with DCM. The organic layers were dried over Na₂SO₄. The organic layers filtered, combined and concentrated. The residue was purified by silica gel chromatography (hexane: EtOAc) to yield the product as a yellow solid (270 mg; 52% yield). ¹H NMR (400 MHz, DMSO-d₆): δ 9.19 (s, 2H), 8.65 (s, 1H), 8.27 (s, 1H), 7.82 (d, 2H, J=7.7 Hz), 7.66 (t, 2H, J=9.06 Hz), 7.50 (d, 1H, J=7.7 Hz), 7.43-7.34 (m, 2H), 7.22 (s, 1H), 7.11 (br s, 1H), 5.99 (q, 1H, J=6.4 Hz), 1.72 (d, 3H, J=6.4 Hz); C₂₄H₁₈Cl₂N₅O₂S: [M+H]⁺ calcd 510.0558, found 510.0564.

### Step B—3-{[(1R)-1-(2-Chlorophenyl)ethyl]oxy}-5-[5-(2-{[3-(dimethylamino)propyl]amino}-5-pyrimidinyl)-1H-benzimidazol-1-yl]-2-thiophenecarboxamide (title compound)

3-{[(1R)-1-(2-Chlorophenyl)ethyl]oxy}-5-[5-(2-chloro-5-pyrimidinyl)-1H-benzimidazol-1-yl]-2-thiophenecarboxamide (80 mg; 0.16 mmol) (prepared according to Example 17, Step A) and 3-(dimethylamino)propylamine (0.5 mL; 4 mmol) were suspended in EtOH (1.0 mL). The reaction was placed in the microwave at 180° C. for 15 min. The reaction mixture was partitioned between EtOAc and water. Separated the layers and extracted the water layer with DCM. The organic layers were dried over Na₂SO₄, filtered, combined and concentrated. The residue was purified by silica gel chromatography (9:1:0.1 DCM:MeOH:ammonium hydroxide) to yield the title compound as light brown solid (37 mg: 41%). ¹H NMR (400 MHz, DMSO-d₆): δ 8.66 (s, 2H), 8.59 (s, 1H), 8.00 (s, 1H), 7.82 (s, 1H), 7.68 (d, 1H, J=7.7 Hz), 7.63 (d, 1H, J=9.7 Hz), 7.56 (d, 1H, J=8.4 Hz), 7.51 (d, 1H, J=7.9 Hz), 7.44-7.31 (m, 3H), 7.21 (s, 1H), 7.11 (br s, 1H), 5.99 (q, 1H, J=6.3 Hz), 3.4-3.2 (m, 2H), 2.28 (t, 2H, J=7.0 Hz), 2.12 (s, 6H), 1.72 (d, 3H, J=6.2 Hz), 1.66 (t, 2H, J=7.1 Hz); C₂₉H₃₁ClN₇O₂S: [M+H]⁺ calcd 576.1948, found 576.1937.

### EXAMPLE 19

### 3-{[(1R)-1-(2-Chlorophenyl)ethyl]oxy}-5-[5-(2-{[2-(dimethylamino)ethyl]amino}-5-pyrimidinyl)-1H-benzimidazol-1-yl]-2-thiophenecarboxamide

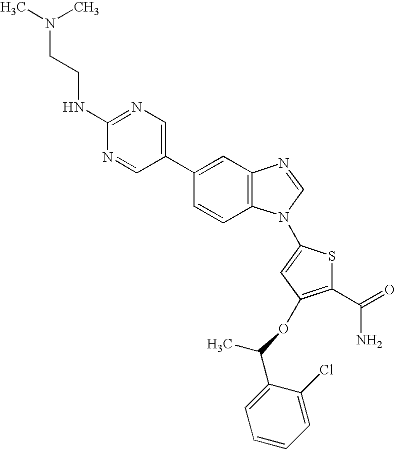

3-{[(1R)-1-(2-Chlorophenyl)ethyl]oxy}-5-[5-(2-chloro-5-pyrimidinyl)-1H-benzimidazol-1-yl]-2-thiophenecarboxamide (50 mg, 0.10 mmol) (prepared according to Example 17, Step B) and N,N′-dimethylethylenediamine (500 L, 7.0 mmol) were suspended in EtOH (1.0 mL). The reaction mixture was placed in the microwave at 180° C. for 15 min. The reaction mixture was partitioned between EtOAc and water. Separated the layers and extracted the water layer with DCM. The organic layers were dried over Na₂SO₄, filtered, combined and concentrated. The residue was purified by silica gel chromatography (9:1:0.1 DCM:MeOH:ammonium hydroxide) to yield the title compound as a yellow solid (50 mg, 91%). ¹H NMR (400 MHz, DMSO-d₆): δ 8.67 (s, 2H), 8.59 (s, 1H), 8.01 (s, 1H), 7.82 (s, 1H), 7.68 (d, 1H, J=7.0 Hz), 7.63 (d, 1H, J=8.6 Hz), 7.56 (d, 1H, J=8.4 Hz), 7.51 (d, 1H, J=7.1 Hz), 7.43-7.34 (m, 2H), 7.21 (s, 1H), 7.10-7.00 (m, 2H), 6.05-5.95 (m, 1H), 3.44-3.3 (m, 2H), 2.24 (t, 2H, J=6.8 Hz), 2.17 (s, 6H), 1.72 (d, 3H, J=5.7 Hz); C₂₈H₂₉ClN₇O₂S: [M+H]⁺ calcd 562.1792, found 562.1790.

### EXAMPLE 20

### 3-{[(1R)-1-(2-Chlorophenyl)ethyl]oxy}-5-[5-(2-{[3-(4-methyl-1-piperazinyl)propyl]amino}-5-pyrimidinyl)-1H-benzimidazol-1-yl]-2-thiophenecarboxamide

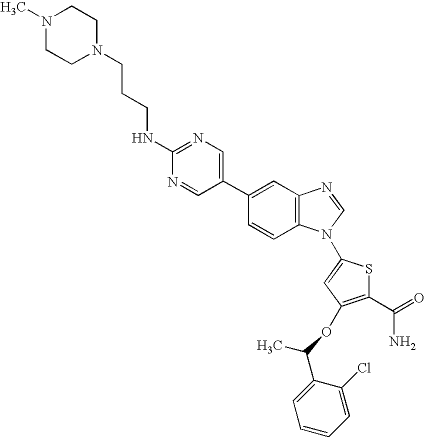

3-{[(1R)-1-(2-Chlorophenyl)ethyl]oxy}-5-[5-(2-chloro-5-pyrimidinyl)-1H-benzimidazol-1-yl]-2-thiophenecarboxamide (88 mg, 0.17 mmol) (prepared according to Example 17, Step B) and 1,(3-aminopropyl)-4-methylpiperazine (500 μL, 2.9 mmol) were suspended in EtOH (1.0 mL). The reaction mixture was placed in the microwave at 180° C. for 15 min. The reaction mixture was partitioned between EtOAc and water. Separated the layers and extracted the water layer with DCM. The organic layers were dried over sodium sulfate, filtered, combined and concentrated. The residue was purified by silica gel chromatography (9:1:0.1 DCM:MeOH:ammonium hydroxide) to yield 30 mg (28%) of the title compound as a yellow foam. ¹H NMR (400 MHz, DMSO-d₆): δ 8.65 (s, 2H), 8.57 (s, 1H), 7.99 (s, 1H), 7.80 (s, 1H), 7.67 (d, 1H, J=7.3 Hz), 7.61 (d, 1H, J=8.1 Hz), 7.55 (d, 1H, J=8.6 Hz), 7.50 (d, 1H, J=7.9 Hz), 7.43-7.30 (m, 3H), 7.19 (s, 1H), 7.10 (brs, 1H), 5.98 (q, 1H, J=6.2 Hz), 3.44-3.3 (m, 2H), 2.40-2.20 (m, 10H), 2.12 (s, 3H), 1.69 (d, 3H, J=6.2 Hz), 1.70-1.60 (m, 2H); C₃₂H₃₆ClN₈O₂S: [M+H]⁺ calcd 631.2370, found 631.2366.

### EXAMPLE 21

### 3-{[(1R)-1-(2-chlorophenyl)ethyl]oxy}-5-(5-{2-[[2-(dimethylamino)ethyl](methyl)amino]-5-pyrimidinyl}-1H-benzimidazol-1-yl)-2-thiophenecarboxamide

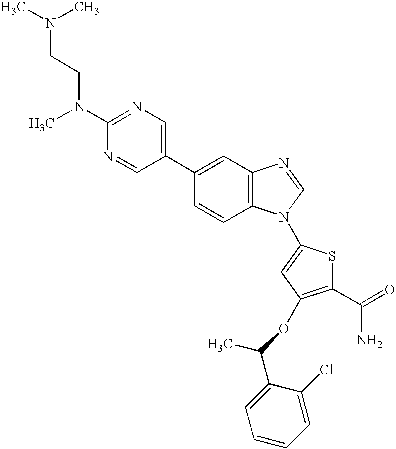

3-{[(1R)-1-(2-Chlorophenyl)ethyl]oxy}-5-[5-(2-chloro-5-pyrimidinyl)-1H-benzimidazol-1-yl]-2-thiophenecarboxamide (80 mg, 0.16 mmol) (prepared according to Example 17, Step B) and N,N,N′-trimethylethylenediamine (500 μL, 6.2 mmol) were suspended in EtOH (0.50 mL). The reaction mixture was placed in the microwave at 180° C. for 15 min. The reaction mixture was partitioned between EtOAc and water. Separated the layers and extracted the water layer with DCM. The organic layers were dried over Na₂SO₄, filtered, combined and concentrated. The residue was purified by silica gel chromatography (9:1:0.1 DCM:MeOH:ammonium hydroxide) to yield 48 mg (53%) of the title compound as a beige solid. ¹H NMR (400 MHz, CDCl₃): δ 8.58 (s, 2H), 7.98 (s, 1H), 7.88 (s, 1H), 7.48-7.41 (m, 4H), 7.36-7.29 (m, 2H), 7.18 (s, 1H), 6.63 (s, 1H), 5.87 (q, 1H, J=6.2 Hz), 5.74 (br s, 1H), 4.00-3.90 (m, 2H), 3.25 (s, 3H), 2.90-2.70 (m, 2H), 2.52 (s, 6H), 1.77 (d, 3H, J=6.4 Hz); C₂₉H₃₁ClN₇O₂S: [M+H]⁺ calcd 576.1948, found 576.1943.

### EXAMPLE 22

### 3-{[(1R)-1-(2-chlorophenyl)ethyl]oxy}-5-[5-(2-{[2-(4-morpholinyl)ethyl]amino}-5-pyrimidinyl)-1H-benzimidazol-1-yl]-2-thiophenecarboxamide

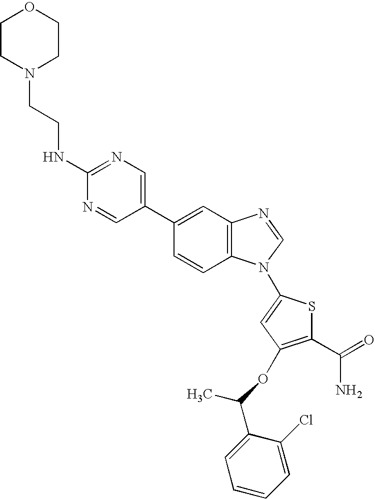

3-{[(1R)-1-(2-Chlorophenyl)ethyl]oxy}-5-[5-(2-chloro-5-pyrimidinyl)-1H-benzimidazol-1-yl]-2-thiophenecarboxamide (75 mg, 0.15 mmol) (prepared according to Example 17, Step B) and 4-(2-aminoethyl)morpholine (500 μL, 3.9 mmol) were suspended in EtOH (1.0 mL). The reaction mixture was placed in the microwave at 180° C. for 15 min. The reaction mixture was partitioned between EtOAc and water. Separated the layers and extracted the water layer with DCM. The organic layers were dried over Na₂SO₄, filtered, combined and concentrated. The residue was purified by silica gel chromatography (9:1:0.1 DCM:MeOH:ammonium hydroxide) to yield 50 mg (55%) of the title compound as a beige solid. ¹H NMR (400 MHz, Acetone-d₆): δ 8.64 (s, 2H), 8.33 (s, 1H), 7.92 (s, 1H), 7.72 (d, 1H, J=7.5 Hz), 7.61-7.55 (m, 3H), 7.51 (d, 1H, J=8.4 Hz), 7.50-7.37 (m, 2H), 7.25 (br s, 1H), 7.11 (s, 1H), 6.91 (br s, 1H), 6.08 (q, 1H, J=6.4 Hz), 3.90-3.40 (m, 8H), 2.80-2.3 (m, 4H), 1.81 (d, 3H, J=6.4 Hz); C₃₀H₃₁ClN₇O₃S: [M+H]⁺ calcd 604.1898, found 604.1898.

### EXAMPLE 23

### 3-{[(1R)-1-(2-chlorophenyl)ethyl]oxy}-5-[5-(3-{[2-(1-Pyrrolidinyl)ethyl]oxy}phenyl)-1H-benzimidazol-1-yl]-2-thiophenecarboxamide

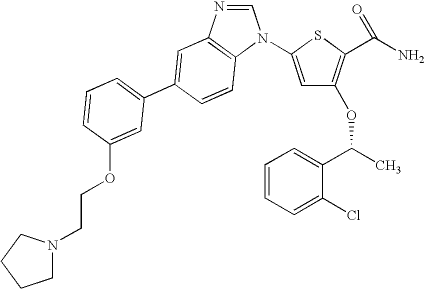

The title compound was prepared from 5-(5-bromo-1H-benzimidazol-1-yl)-3-{[(1R)-1-(2-chlorophenyl)ethyl]oxy}thiophene-2-carboxamide (100 mg, 0.21 mmol) (prepared according to Example 10, Step D) by a procedure analogous to Example 1, Step E. The title compound was obtained in 92% yield (113 mg). ¹H NMR (300 MHz, DMSO-d₆) [symbol] 8.64 (s, 1H), 8.01 (s, 1H), 7.86 (s, 1H), 7.74-7.39 (m, 9H), 7.27 (s, 1H), 7.16-7.10 (m, 2H), 6.07-6.01 (m, 1H), 4.30-4.21 (m, 2H), 3.20-2.81 (m, 6H), 1.85 (s, 4H), 1.77 (d, 3H, J=6.3 Hz); MS (ESI) m/z 587 (M⁺+H).

### EXAMPLE 24

### 3-{[(1R)-1-(2-chlorophenyl)ethyl]oxy}-5-(5-[4-[(4-morpholinylacetyl)amino]phenyl]-1H-benzimidazol-1-yl)-2-thiophenecarboxamide

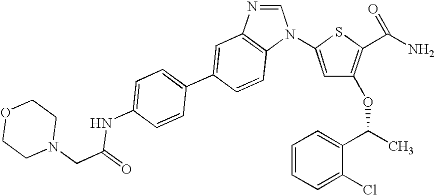

The title compound was prepared from 5-(5-bromo-1H-benzimidazol-1-yl)-3-{[(1R)-1-(2-chlorophenyl)ethyl]oxy}thiophene-2-carboxamide (100 mg, 0.21 mmol) (prepared according to Example 10, Step D) by a procedure analogous to Example 1, Step E. The title compound was obtained as a solid (148 mg). ¹H NMR (400 MHz, DMSO-d₆) [symbol] 9.81 (s, 1H), 8.62 (s, 1H), 7.98 (s, 2H), 7.81 (s, 1H), 7.70-7.67 (m, 2H), 7.64 (s, 1H), 7.52-7.36 (m, 6H), 7.24 (s, 1H), 7.60 (s, 1H), 6.02-5.97 (m, 1H), 3.64 (t, 4H, J=4.4, 8.8 Hz), 3.14 (s, 2H), 2.51 (t, 4H, J=4.4, 8.8 Hz), 1.73 (d, 3H, J=6.4 Hz); MS (ESI) m/z 616 (M⁺+H).

### EXAMPLE 25

### 5-[5-(6-{[2-(dimethylamino)ethyl]amino}-3-pyridinyl)-1H-benzimidazol-1-yl]-3-({(1R)-1-[2-(trifluoromethyl)phenyl]ethyl}oxy)-2-thiophenecarboxamide

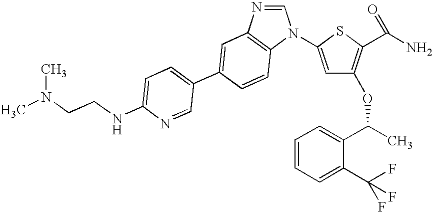

### Step A—5-[5-(6-fluoro-3-pyridinyl)-1H-benzimidazol-1-yl]-3-({(1R)-1-[2-(trifluoromethyl)phenyl]ethyl}oxy)-2-thiophenecarboxamide

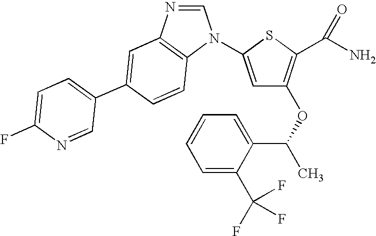

The title compound was prepared from 5-(5-Bromo-1H-benzimidazol-1-yl)-3-({(1R)-1-[2-(trifluoromethyl)phenyl]-ethyl}oxy)thiophene-2-carboxamide (1.0 g, 2.0 mmol) (prepared according to Example 10, Step D) and 2-fluoropyridine-5-boronic acid (0.33 g, 2.35 mmol) by a procedure analogous to Example 1, Step E. The title compound was obtained in 70% yield (700 mg). MS (ESI): 527.0 [M+H]⁺.

### Step B—5-[5-(6-{[2-(dimethylamino)ethyl]amino}-3-pyridinyl)-1H-benzimidazol-1-yl]-3-({(1R)-1-[2-(trifluoromethyl)phenyl]ethyl}oxy)-2-thiophenecarboxamide (title compound)

The title compound was prepared from 5-[5-(6-fluoropyridin-3-yl)-1H-benzimidazol-1-yl]-3-({(1R)-1-[2-(trifluoromethyl)phenyl]ethyl}oxy)-2-thiophenecarboxamide (300 mg, 0.58 mmol) (prepared according to Example 25) by a procedure analogous to Example 7. The title compound was obtained in 38% yield (130 mg). ¹H NMR (400 MHz, CD₃OD) δ 8.34 (s, 1H), 8.26 (d, J=2.6 Hz, 1H), 7.86-7.84 (m, 2H), 7.78 (dd, J=8.6, 2.3 Hz, 2H), 7.71 (t, J=7.6 Hz, 1H), 7.53 (m, 2H), 7.44 (d, J=8.6 Hz, 1H), 6.97 (s, 1H), 6.65 (d, J=8.9 Hz, 1H), 6.04-5.98 (m, 1H), 3.50 (m, 2H), 2.71 (t, J=6.6 Hz, 2H), 2.41 (s, 6H), 2.26 (s, 1H), 1.81 (d, J=6.4 Hz, 3H); MS (ESI) m/z 595.29 (M+H)⁺.

### EXAMPLE 26

### 5-[5-(6-{[2-(methylamino)ethyl]amino}-3-pyridinyl)-1H-benzimidazol-1-yl]-3-({(1R)-1-[2-(trifluoromethyl)phenyl]ethyl}oxy)-2-thiophenecarboxamide

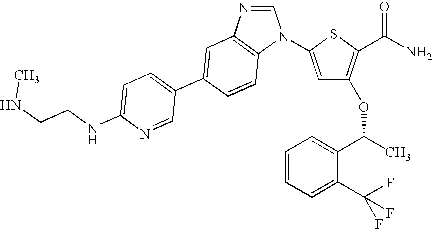

### Step A—methyl 5-[5-(6-chloro-3-pyridinyl)-1H-benzimidazol-1-yl]-3-({(1R)-1-[2-(trifluoromethyl)phenyl]ethyl}oxy)-2-thiophenecarboxylate

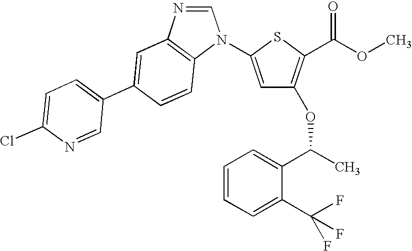

The title compound was prepared from methyl 5-(5-bromo-1H-benzimidazol-1-yl)-3-({(1R)-1-[2-(trifluoromethyl)phenyl]ethyl}oxy)-2-thiophenecarboxylate (0.8 g, 1.5 mmol) (prepared according to Example 1, Step C) and 2-chloro-5-(4,4,5,5-tetramethyl-1,3,2-dioxaborolan-2-yl)pyridine (0.55 g, 2.3 mmol) by a procedure analogous to Example 1, Step E. The title compound was obtained in 46% yield (390 mg). MS (ESI): 558.1 [M+H]⁺.

### Step B—methyl 5-{5-[6-({2-[{[(1,1-dimethylethyl)oxy]carbonyl}-(methyl)amino]ethyl}amino)-3-pyridinyl]-1H-benzimidazol-1-yl}-3-({(1R)-1-[2-(trifluoromethyl)phenyl]ethyl}oxy)-2-thiophenecarboxylate

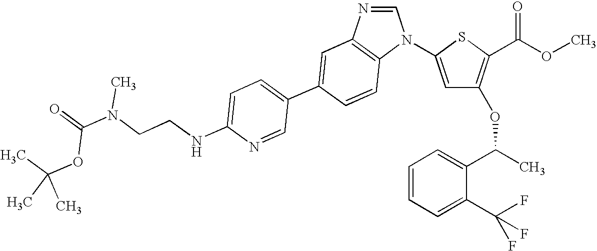

The title compound was prepared from methyl 5-[5-(6-chloro-3-pyridinyl)-1H-benzimidazol-1-yl]-3-({(1R)-1-[2-(trifluoromethyl)phenyl]ethyl}oxy)-2-thiophenecarboxylate (150 mg, 0.27 mmol) (prepared according to Example 26, Step A) and 1,1-dimethylethyl (2-aminoethyl)methylcarbamate (100 mg, 0.57 mmol) by a procedure analogous to Example 3, Step B. The title compound was obtained in 30% yield (57 mg). MS (ESI): 696.3 [M+H]⁺.

### Step C—1,1-Dimethylethyl {2-[(5-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]ethyl}methylcarbamate

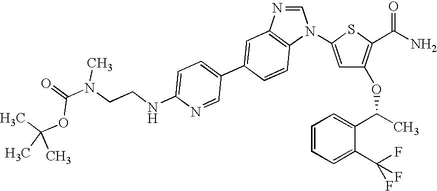

The title compound was prepared from methyl 5-{5-[6-({2-[{[(1,1-dimethylethyl)oxy]carbonyl}(methyl)amino]ethyl}amino)-3-pyridinyl]-1H-benzimidazol-1-yl}-3-({(1R)-1-[2-(trifluoromethyl)phenyl]ethyl}oxy)-2-thiophenecarboxylate (57 mg, 0.08 mmol) (prepared according to Example 26, Step B) by a procedure analogous to Example 1, Step D. The title compound was obtained in 52% yield (29 mg). MS (ESI): 581.3 [M+H]⁺.

### Step D—5-[5-(6-{[2-(methylamino)ethyl]amino}-3-pyridinyl)-1H-benzimidazol-1-yl]-3-({(1R)-1-[2-(trifluoromethyl)phenyl]ethyl}oxy)-2-thiophenecarboxamide (title compound)

The title compound was prepared from 1,1-dimethylethyl {2-[(5-{1-[5-(aminocarbonyl)-4-({(1R)-1-[2-(trifluoromethyl)phenyl]ethyl}oxy)-2-thienyl]-1H-benzimidazol-5-yl}-2-pyridinyl)amino]ethyl}methylcarbamate (29 mg, 0.043 mmol) (prepared according to Example 26, Step C) by a procedure analogous to Example 3, Step C. The title compound was obtained in 96% yield (27 mg). ¹H NMR (300 MHz, CD₃OD) [symbol] 9.32 (s, 1H), 8.92-8.85 (m, 1H), 8.49 (dd, J=8.4, 1.9 Hz, 1H), 8.38 (s, 1H), 8.17 (s, 1H), 7.96-7.70 (m, 4H), 7.65-7.56 (m, 1H), 7.38 (d, J=9.4 Hz, 1H), 7.22 (s, 1H), 6.13-6.05 (m, 1H), 3.94 (t, J=6.2 Hz, 2H), 3.45 (t, J=6.4 Hz, 2H), 2.87 (s, 3H), 1.89 (d, J=5.5 Hz, 3H); MS (ESI) m/z 581.26 (M+H)⁺.

### EXAMPLE 27

### 3-{[(1R)-1-(2-chlorophenyl)ethyl]oxy}-5-(5-[6-[[2-(dimethylamino)ethyl](methyl)amino]-3-pyridinyl]-1H-benzimidazol-1-yl)-2-thiophenecarboxamide

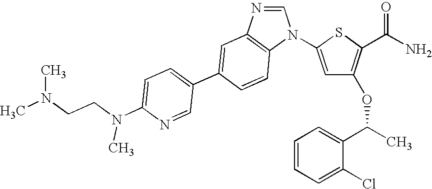

### Step A—3-{[(1R)-1-(2-Chlorophenyl)ethyl]oxy}-5-[5-(6-fluoropyridin-3-yl)-1H-benzimidazol-1-yl]thiophene-2-carboxamide

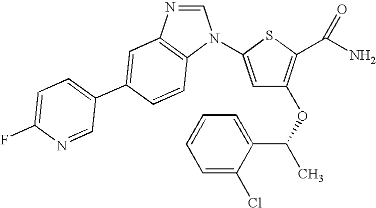

The title compound was prepared from 5-(5-bromo-1H-benzimidazol-1-yl)-3-{[(1R)-1-(2-chlorophenyl)ethyl]oxy}thiophene-2-carboxamide (3.35 g, 7.03 mmol) (prepared according to Example 1, Step D) and 2-fluoropyridine-5-boronic acid (1.19 g, 8.43 mmol) by a procedure analogous to Example 1, Step E. The title compound was prepared in 89% yield (3.09 g). ¹H NMR (400 MHz, CDCl₃): δ 8.47 (s, 1H), 8.02 (m, 2H), 7.49 (m, 4H), 7.34 (m, 2H), 7.21 (br s, 1H), 7.03 (m, 1H), 6.67 (s, 1H), 5.89 (m, 1H), 5.76 (br s, 1H), 1.79 (d, 3H, J=5.6 Hz); MS (ESI): 493.1 [M+H]⁺.

### Step B—3-{[(1R)-1-(2-chlorophenyl)ethyl]oxy}-5-(5-{6-[[2-(dimethylamino)ethyl](methyl)amino]-3-pyridinyl}-1H-benzimidazol-1-yl)-2-thiophenecarboxamide (title compound)

The title compound was prepared from 3-{[(1R)-1-(2-chlorophenyl)ethyl]oxy}-5-[5-(6-fluoropyridin-3-yl)-1H-benzimidazol-1-yl]thiophene-2-carboxamide (50 mg, 0.10 mmol) by a procedure analogous to Example 7. The title compound was obtained in 71% yield (41 mg). ¹H NMR (400 MHz, CDCl₃) δ 8.41 (d, J=2.5 Hz, 1H), 7.96 (s, 1H), 7.91 (s, 1H), 7.75 (dd, J=8.7, 2.5 Hz, 1H), 7.50-7.41 (m, 4H), 7.36-7.27 (m, 2H), 7.24 (s, 1H), 6.63-6.61 (m, 2H), 5.87 (m, 1H), 5.71 (s, 1H), 4.03-3.97 (m, 4H), 3.13 (s, 3H), 2.67 (s, 6H), 1.77 (d, J=5.9 Hz, 3H); MS (ESI) m/z 575.31 [M+H]⁺.

### EXAMPLE 28

### 3-{[(1R)-1-(2-chlorophenyl)ethyl]oxy}-5-(5-[6-[(2-hydroxyethyl)amino]-3-pyridinyl]-1H-benzimidazol-1-yl)-2-thiophenecarboxamide

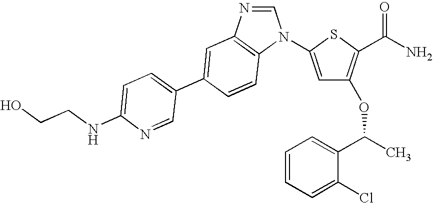

The title compound was prepared from 3-{[(1R)-1-(2-chlorophenyl)ethyl]oxy}-5-[5-(6-fluoropyridin-3-yl)-1H-benzimidazol-1-yl]thiophene-2-carboxamide (50 mg, 0.10 mmol) (prepared according to Example 27, Step A) by a procedure analogous to Example 7. The title compound was obtained in 59% yield (32 mg). ¹H NMR (400 MHz, CDCl₃) δ 8.32 (d, J=2.6 Hz, 1H), 7.97 (s, 1H), 7.89 (s, 1H), 7.69 (dd, J=8.4, 2.4 Hz, 1H), 7.48-7.41 (m, 4H), 7.36-7.27 (m, 3H), 7.18 (s, 1H), 6.62 (s, 1H), 6.56 (d, J=8.4 Hz, 1H), 5.87 (m, 1H), 5.71 (s, 1H), 3.84 (t, J=4.7 Hz, 2H), 3.57 (m, 2H), 1.77 (d, J=6.7 Hz, 3H); MS (ESI) m/z 534.19 [M+H]⁺.

### EXAMPLE 29

### 3-{[(1R)-1-(2-chlorophenyl)ethyl]oxy}-5-[5-(6-{[3-(dimethylamino)propyl]amino}-3-pyridinyl)-1H-benzimidazol-1-yl]-2-thiophenecarboxamide

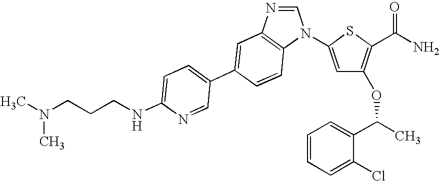

The title compound was prepared from 3-{[(1R)-1-(2-chlorophenyl)ethyl]oxy}-5-[5-(6-fluoropyridin-3-yl)-1H-benzimidazol-1-yl]thiophene-2-carboxamide (100 mg, 0.20 mmol) (prepared according to Example 27, Step A) by a procedure analogous to Example 7. The title compound was obtained in 87% yield (104 mg). ¹H NMR (400 MHz, CDCl₃) δ 8.35 (d, J=2.5 Hz, 1H), 7.96 (s, 1H), 7.90 (s, 1H), 7.67 (dd, J=8.6, 2.6 Hz, 1H), 7.47-7.41 (m, 3H), 7.36-7.27 (m, 2H), 7.18 (s, 1H), 6.62 (s, 1H), 6.49 (d, J=8.6 Hz, 1H), 5.87 (m, 1H), 5.71 (s, 1H), 5.63 (s, 1H), 3.45-3.38 (m, 2H), 2.51 (t, J=6.8 Hz, 2H), 2.32 (s, 6H), 1.89-1.82 (m, 2H), 1.77 (d, J=7.2 Hz, 3H); MS [ESI] m/z 575.44 (M+H)⁺.

### EXAMPLE 30

### 3-{[(1R)-1-(2-chlorophenyl)ethyl]oxy}-5-[5-(6-{[2-(dimethylamino)ethyl]amino}-3-pyridinyl)-1H-benzimidazol-1-yl]-2-thiophenecarboxamide

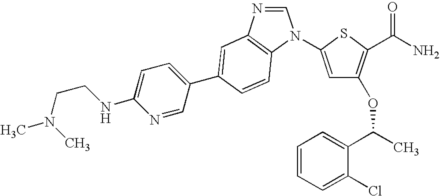

The title compound was prepared from 3-{[(1R)-1-(2-chlorophenyl)ethyl]oxy}-5-[5-(6-fluoropyridin-3-yl)-1H-benzimidazol-1-yl]thiophene-2-carboxamide (100 mg, 0.20 mmol) (prepared according to Example 27, Step A) by a procedure analogous to Example 7. The title compound was obtained in 89% yield (104 mg). ¹H NMR (400 MHz, CDCl₃) δ 8.36 (d, J=2.6 Hz, 1H), 7.96 (s, 1H), 7.90 (s, 1H), 7.67 (dd, J=8.7, 2.4 Hz, 1H), 7.47-7.41 (m, 3H), 7.35-7.27 (m, 2H), 7.18 (s, 1H), 6.62 (s, 1H), 6.52 (d, J=8.4 Hz, 1H), 5.87 (q, J=6.5 Hz, 1H), 5.73 (s, 1H), 3.45 (q, J=5.8 Hz, 2H), 2.63 (t, J=6.2 Hz, 2H), 2.32 (s, 6H), 2.23 (m, 2H), 1.77 (d, J=6.0 Hz, 3H); MS [ESI] m/z 561.36 (M+H)⁺.

### EXAMPLE 31

### 3-{[(1R)-1-(2-chlorophenyl)ethyl]oxy}-5-[5-(6-{[3-(4-methyl-1-piperazinyl)propyl]amino}-3-pyridinyl)-1H-benzimidazol-1-yl]-2-thiophenecarboxamide

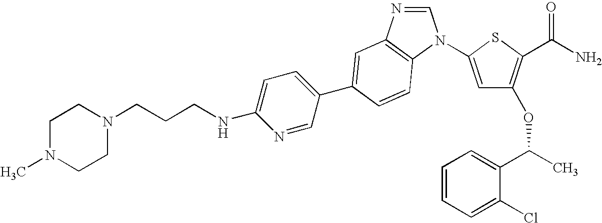

The title compound was prepared from 3-{[(1R)-1-(2-chlorophenyl)ethyl]oxy}-5-[5-(6-fluoropyridin-3-yl)-1H-benzimidazol-1-yl]thiophene-2-carboxamide (500 mg, 1.0 mmol) (prepared according to Example 27, Step A) by a procedure analogous to Example 7. The title compound was obtained in 87% yield (545 mg). ¹H NMR (400 MHz, CDCl₃) δ 8.35 (d, J=2.2 Hz, 1H), 7.96 (s, 1H), 7.90 (s, 1H), 7.67 (dd, J=8.5, 2.5 Hz, 1H), 7.47-7.41 (m, 3H), 7.35-7.27 (m, 2H), 7.18 (s, 1H), 6.46 (d, J=8.5 Hz, 1H), 5.87 (m, 1H), 5.77 (s, 1H), 3.40 (m, 2H), 2.66-2.39 (m, 10H), 4.14 (s, 3H), 1.83-1.77 (m, 2H), 1.75 (d, J=5.0 Hz, 3H); MS (ESI) m/z 630.29 [M+H]⁺.

### EXAMPLE 32

### 3-{[(1R)-1-(2-chlorophenyl)ethyl]oxy}-5-[5-(6-{[2-(4-morpholinyl)ethyl]amino}-3-pyridinyl)-1H-benzimidazol-1-yl]-2-thiophenecarboxamide

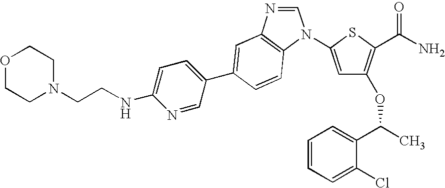

The title compound was prepared from 3-{[(1R)-1-(2-chlorophenyl)ethyl]oxy}-5-[5-(6-fluoropyridin-3-yl)-1H-benzimidazol-1-yl]thiophene-2-carboxamide (100 mg, 0.20 mmol) (prepared according to Example 27, Step A) by a procedure analogous to Example 7. The title compound was obtained in 69% yield (84 mg). ¹H NMR (400 MHz, CDCl₃) δ 8.37 (d, J=2.3 Hz, 1H), 7.96 (s, 1H), 7.90 (s, 1H), 7.70 (dd, J=8.5, 2.9 Hz, 2H), 7.48-7.41 (m, 3H), 7.36-7.26 (m, 2H), 7.18 (s, 1H), 6.62 (s, 1H), 6.50 (d, J=8.4 Hz, 1H), 5.87 (q, J=6.4 Hz, 1H), 5.73 (s, 1H), 5.17 (t, J=5.3 Hz, 1H), 3.74-3.67 (m, 4H), 3.42-3.38 (m, 2H), 2.64 (t, J=5.7 Hz, 2H), 2.51-2.43 (m, 4H), 1.77 (d, J=4.3 Hz, 3H); MS (ESI) m/z 603.25 [M+H]⁺.

### EXAMPLE 33

### 3-{[(1R)-1-(2-chlorophenyl)ethyl]oxy}-5-(5-[6-[[3-(dimethylamino)propyl](methyl)amino]-3-pyridinyl]-1H-benzimidazol-1-yl)-2-thiophenecarboxamide

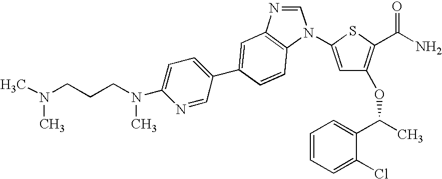

The title compound was prepared from 3-{[(1R)-1-(2-chlorophenyl)ethyl]oxy}-5-[5-(6-fluoropyridin-3-yl)-1H-benzimidazol-1-yl]thiophene-2-carboxamide (100 mg, 0.20 mmol) (prepared according to Example 27, Step A) by a procedure analogous to Example 7. The title compound was obtained in 67% yield (79 mg). ¹H NMR (400 MHz, CDCl₃) δ 8.43 (d, J=2.4 Hz, 1H), 7.96 (s, 1H), 7.91 (s, 1H), 7.71 (dd, J=9.1, 2.8 Hz, 1H), 7.49-7.40 (m, 4H), 7.35-7.27 (m, 2H), 7.19 (s, 1H), 6.62-6.58 (m, 2H), 5.87 (q, J=6.4 Hz, 1H), 5.78 (s, 1H), 3.62 (t, J=7.0 Hz, 2H), 3.08 (s, 3H), 2.40 (m, 2H), 2.29 (s, 6H), 1.76 (d, J=6.6 Hz, 3H); MS (APCI) m/z 589.54 [M+H]⁺.

### EXAMPLE 34

### 3-{[(1R)-1-(2-chlorophenyl)ethyl]oxy}-5-[5-(6-{[3-(4-methyl-1-piperazinyl)propyl]amino}-3-pyridazinyl)-1H-benzimidazol-1-yl]-2-thiophenecarboxamide

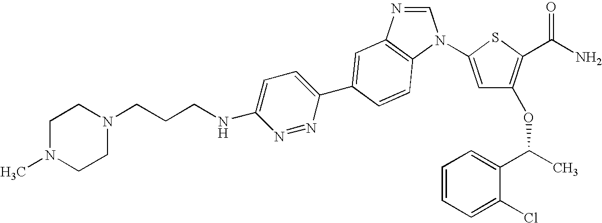

### Step A—3-{[(1R)-1-(2-chlorophenyl)ethyl]oxy}-5-[5-(6-chloro-3-pyridazinyl)-1H-benzimidazol-1-yl]-2-thiophenecarboxamide

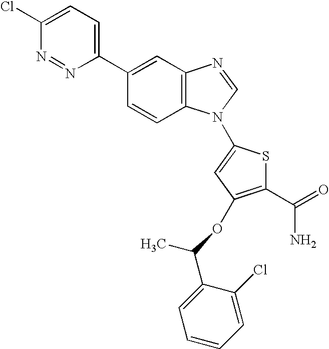

The title compound was prepared from 3-{[(1R)-1-(2-chlorophenyl)ethyl]oxy}-5-[5-(4,4,5,5-tetramethyl-1,3,2-dioxaborolan-2-yl)-1H-benzimidazol-1-yl]thiophene-2-carboxamide (175 mg, 0.33 mmol) (prepared according to Example 17, Step A) and 3,6-dichloropyridazine (98 mg, 0.66 mmol) by a procedure analogous to Example 16, Step B (31% yield, 52 mg). MS (ESI): 510.7 [M+H]⁺.

### Step B—3-{[(1R)-1-(2-chlorophenyl)ethyl]oxy}-5-[5-(6-{[3-(4-methyl-1-piperazinyl)propyl]amino}-3-pyridazinyl)-1H-benzimidazol-1-yl]-2-thiophenecarboxamide (title compound)

The title compound was prepared from 3-{[(1R)-1-(2-chlorophenyl)ethyl]oxy}-5-[5-(6-chloro-3-pyridazinyl)-1H-benzimidazol-1-yl]-2-thiophenecarboxamide (52 mg, 0.10 mmol) and [3-(4-methyl-1-piperazinyl)propyl]amine (0.3 mL, 1.8 mmol) by a procedure analogous to Example 16, Step C. The title compound was isolated as a yellow solid (42% yield, 27 mg). ¹H NMR (400 MHz, CDCl₃): [symbol] 8.23 (s, 1H), 8.19 (d, 1H, J=8.0 Hz), 8.00 (s, 1H), 7.65 (d, 1H, J=8.0 Hz), 7.51-7.42 (m, 2H), 7.40 (d, 1H, J=8.0 Hz), 7.38-7.28 (m, 2H), 7.20 (br s, 1H), 6.72 (d, 1H, J=8.0 Hz), 6.65 (s, 1H), 6.22 (t, 1H, J=4.0 Hz), 6.05 (br s, 1H), 5.87 (q, 1H, J=4.0 Hz), 3.59 (d, 1H, J=4.0 Hz), 2.62-2.41 (m, 8H), 2.32 (s, 3H), 2.28 (m, 2H), 2.01 (br s, 1H), 1.90-1.84 (m, 2H), 1.77 (d, 3H, J=4.0 Hz); MS (ESI): 632.1 [M+H]⁺.

### Biological Examples

### I. Assay for Inhibition of PLK1

A. Preparation of 6× N-Terminal His-Tagged PLK Kinase Domain

6×N-terminal His-tagged PLK kinase domain (amino acids 21-346 preceded by MKKGHHHHHHD) SEQ ID: No. 1. was prepared from baculovirus infected T. ni cells under polyhedrin promoter control. All procedures were performed at 4° C. Cells were lysed in 50 mM HEPES, 200 mM NaCl, 50 mM imidazole, 5% glycerol; pH 7.5. The homogenate was centrifuged at 14K rpm in a SLA-1500 rotor for 1 hr and the supernatant filtered through a 1.2 micron filter. The supernatant was loaded onto a Nickel chelating Sepharose (Amersham Pharmacia) column and washed with lysis buffer. Protein was eluted using 20%, 30% and 100% buffer B steps where buffer B was 50 mM HEPES, 200 mM NaCl, 300 mM imidazole, 5% glycerol; pH 7.5. Fractions containing PLK were determined by SDS-PAGE. Fractions containing PLK were diluted five-fold with 50 mM HEPES, 1 mM DTT, 5% glycerol; pH 7.5, then loaded on an SP Sepharose (Amersham Pharmacia) column. After washing the column with 50 mM HEPES, 1 mM DTT, 5% glycerol; pH 7.5, PLK was step eluted with 50 mM HEPES, 1 mM DTT, 500 mM NaCl; 5% glycerol; pH 7.5. PLK was concentrated using a 10 kDa molecular weight cutoff membrane and then loaded onto a Superdex 200 gel filtration (Amersham Pharmacia) column equilibrated in 25 mM HEPES, 1 mM DTT, 500 mM NaCl, 5% glycerol; pH 7.5. Fractions containing PLK were determined by SDS-PAGE. PLK was pooled, aliquoted and stored at −80° C. Samples were quality controlled using mass spectrometry, N-terminal sequencing and amino acid analysis.

B. Enzyme Activity +/− Inhibitors was Determined as Follows:

All measurements were obtained under conditions where signal production increased linearly with time and enzyme. Test compounds were added to white 384-well assay plates (0.1 μL for 10 μL and some 20 μL assays, 1 μL for some 20 μL assays) at variable known concentrations in 100% DMSO. DMSO (1-5% final, as appropriate) and EDTA (65 mM in reaction) were used as controls. Reaction Mix was prepared as follows at 22° C.:
25 mM HEPES, pH 7.2
15 mM MgCl₂
1 μM ATP
0.05 μCi/well ³³P-γ ATP (10 Ci/mMol)
1 μM substrate peptide (Biotin-Ahx-SFNDTLDFD) SEQ ID:No. 2.
0.15 mg/mL BSA
1 mM DTT
2 nM PLK1 kinase domain (added last)

Reaction Mix (10 or 20 μL) was quickly added to each well immediately following addition of enzyme via automated liquid handlers and incubated 1-1.5 h at 22° C. The 20 μL enzymatic reactions were stopped with 50 μL of stop mix (50 mM EDTA, 4.0 mg/mL Streptavidin SPA beads in Standard Dulbecco's PBS (without Mg²⁺ and Ca²⁺), 50 μM ATP) per well. The 10 μL reactions were stopped with 10 μL of stop mix (50 mM EDTA, 3.0 mg/mL Streptavidin-coupled SPA Imaging Beads (“LeadSeeker”) in Standard Dulbecco's PBS (without Mg²⁺ and Ca²⁺), 50 μM ATP) per well. Plates were sealed with clear plastic seals, spun at 500×g for 1 min or settled overnight, and counted in Packard TopCount for 30 seconds/well (regular SPA) or imaged using a Viewlux imager (LeadSeeker SPA). Signal above background (EDTA controls) was converted to percent inhibition relative to that obtained in control (DMSO-only) wells.

C. Results

The data obtained is reported in Table 1 below. In Table 1, +=pIC₅₀<6; ++=pIC₅₀ 6-7; +++=pIC₅₀>7.

### II. Cell-Titer Glo Cell Growth Inhibition Assay

A549-L (human lung), Colo 205 (human colon), HCT-116 (human colon), HT29 (human colon), MX-1 (human breast), SKOV3 (human ovary) and P388 (murine leukemia) cell lines were cultured in RPM11640 containing 10% fetal bovine serum at 37° C. in a 5% CO₂ incubator. Cell lines were split into T75 flasks (Falcon #353136) two to three days prior to assay set up at density which yields approximately 70-80% confluency at time of harvest for assay. Cells were harvested using 0.25% trypsin with EDTA (Glibco/Invitrogen #25200-056), Cell counts were performed on cell suspensions using Trypan Blue exclusion staining, cells were then plated in 105 □L of culture media per well at the following densities in 96 well Costar #3916 black flat bottom plates: A549-L 500 cells/well, Colo205 500 cells/well, HCT-116 500 cells/well, HT29 500 cells/well, MX-1 500 cells/well, SKOV3 500 cells/well and P388 250 cells/well. All plates were placed at 5% CO₂ 37° C. overnight and then test compounds were added the following day. One plate of each cell type was treated with CellTiter-Glo (Promega #G7573) for a day 0 proliferation measurement and read as described below. The test compounds were prepared in clear U bottom polypropylene 96 well plates (Falcon #35-1190 with consecutive two fold dilutions. 9 uL of these dilutions was added into each well of the cell plates. The final concentration of DMSO in all wells was 0.15%. Cells were incubated at 37° C., 5% CO₂ for 72 h. Following 72 h of incubation with compounds each plate was developed and read. CellTiter-Glo reagent was added to assay plates using a volume equivalent to the cell culture volume in the wells. Plates were shaken for approximately 2 min and incubated at rt for approximately 15 min and the chemiluminescent signal was read on the Victor V or Envison 2100 reader. Percent inhibition of cell growth was expressed as percent proliferation relative to 100% proliferation (DMSO control). Concentration of compound that inhibited 50% of cell growth (IC₅₀) was analyzed by 4 or 6 parameter fit of data using XLfit, value of no cell addition was substracted from all samples for background. The data are shown in Table 1 below.

TABLE 1

| Example | PLK1 pIC₅₀ | HCT116 IC₅₀ |
|---|---|---|
| 1 | +++ | +++ |
| 2 | +++ | +++ |
| 3 | +++ | +++ |
| 4 | +++ | +++ |
| 5 | +++ | +++ |
| 6 | +++ | +++ |
| 7 | +++ | +++ |
| 8 | +++ | +++ |
| 9 | +++ | +++ |
| 10 | +++ | +++ |
| 11 | +++ | +++ |
| 12 | +++ | +++ |
| 13 | +++ | +++ |
| 14 | +++ | +++ |
| 15 | +++ | +++ |
| 16 | +++ | +++ |
| 17 | +++ | +++ |
| 18 | +++ | +++ |
| 19 | +++ | +++ |
| 20 | +++ | +++ |
| 21 | +++ | +++ |
| 22 | +++ | +++ |
| 23 | +++ | +++ |
| 24 | +++ | +++ |
| 25 | +++ | +++ |
| 26 | +++ | +++ |
| 27 | +++ | +++ |
| 28 | +++ | +++ |
| 29 | +++ | +++ |
| 30 | +++ | +++ |
| 31 | +++ | +++ |
| 32 | +++ | +++ |
| 33 | +++ | +++ |
| 34 | +++ | ++ |

In Table 1,
+ = IC₅₀ > 1 μM;
++ = IC₅₀ 0.5-1 μM:
+++ = IC₅₀ < 0.5 μM.

What is claimed is:

1. A compound of formula (I): 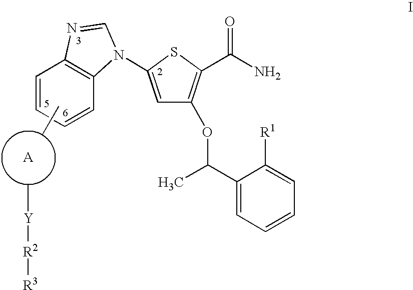
wherein
R¹ is F, Cl, CH₃ or CF₃;
Ring A is selected from phenyl and 5-6 membered heteroaryl having 1 or 2 heteroatoms selected from N, O and S;
Y is selected from —O—, —N(R⁴)—, —N(R⁴)—C(O)— and —N(R⁴)—R²—OC(O)—;
R² is linear or branched C₁₋₄alkylene optionally substituted once by —OH;
R³ is selected from —OR⁴, —N(R⁴)₂ and Het;
each R⁴ is the same or different and is independently H or C₁₋₄alkyl;
Het is a 5-6 member heterocycle having 1 or 2 heteroatoms selected from N, O and S and optionally substituted by 1 or 2 substituents selected from halo, C₁₋₄alkyl, oxo, —OR⁴, —C(O)R⁴, —C(O)₂R⁴, —SO₂R⁴ and —CN;
or a pharmaceutically acceptable salt thereof.

2. The compound according to claim 1, wherein Ring A is phenyl, pyridine or pyrimidine.

3. The compound according to claim 1, wherein Y is —N(H)—, —N(CH₃)— or —N(H)—C(O)—.

4. The compound according to claim 1, wherein R³ is —N(R⁴)₂ or Het.

5. The compound according to claim 1, wherein Het is pyrrolidine, piperizine, or morpholine, each optionally substituted 1 or 2 times by C₁₋₄alkyl.

6. An enantiomerically enriched compound according to claim 1, having the stereochemistry depicted in formula (I-1): 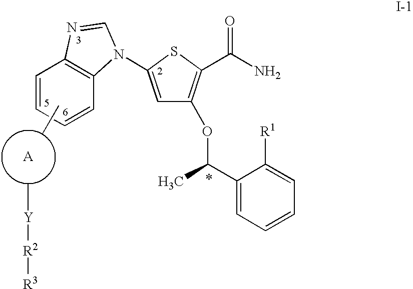
wherein * indicates the chiral carbon and all variables are as defined in claim 1.

7. A compound selected from
5-[5-(2-{[2-(Dimethylamino)ethyl]amino}-4-pyridinyl)-1H-benzimidazol-1-yl]-3-({(1R)-1-[2-(trifluoromethyl)phenyl]ethyl}oxy)-2-thiophenecarboxamide;
5-[5-(2-{[2-(Methylamino)ethyl]amino}-4-pyridinyl)-1H-benzimidazol-1-yl]-3-({(1R)-1-[2-(trifluoromethyl)phenyl]ethyl}oxy)-2-thiophenecarboxamide;
5-(5-{2-[(N,N-dimethylglycyl)amino]-4-pyridinyl}-1H-benzimidazol-1-yl)-3-({(1R)-1-[2-(trifluoromethyl)phenyl]ethyl}oxy)-2-thiophenecarboxamide;
5-{5-[2-(Glycylamino)-4-pyridinyl]-1H-benzimidazol-1-yl}-3-({(1R)-1-[2-(trifluoromethyl)phenyl]ethyl}oxy)-2-thiophenecarboxamide;
5-[5-(2-{[3-(Dimethylamino)propyl]amino}-4-pyridinyl)-1H-benzimidazol-1-yl]-3-({(1R)-1-[2-(trifluoromethyl)phenyl]ethyl}oxy)-2-thiophenecarboxamide;
5-(5-{2-[(2-Aminoethyl)amino]-4-pyridinyl}-1H-benzimidazol-1-yl)-3-({(1R)-1-[2-(trifluoromethyl)phenyl]ethyl}oxy)-2-thiophenecarboxamide;
5-(5-{2-[[2-(Dimethylamino)ethyl](methyl)amino]-4-pyridinyl}-1H-benzimidazol-1-yl)-3-({(1R)-1-[2-(trifluoromethyl)phenyl]ethyl}oxy)-2-thiophenecarboxamide;
5-(5-{2-[[3-(Dimethylamino)propyl](methyl)amino]-4-pyridinyl}-1H-benzimidazol-1-yl)-3-({(1R)-1-[2-(trifluoromethyl)phenyl]ethyl}oxy)-2-thiophenecarboxamide;
5-[5-(2-{[3-(4-Methyl-1-piperazinyl)propyl]amino}-4-pyridinyl)-1H-benzimidazol-1-yl]-3-({(1R)-1-[2-(trifluoromethyl)phenyl]ethyl}oxy)-2-thiophenecarboxamide;
3-{[(1R)-1-(2-chlorophenyl)ethyl]oxy}-5-[5-(2-{[2-(dimethylamino)ethyl]amino}-4-pyridinyl)-1H-benzimidazol-1-yl]-2-thiophenecarboxamide;
3-{[(1R)-1-(2-chlorophenyl)ethyl]oxy}-5-[5-(2-{[3-(4-methyl-1-piperazinyl)propyl]amino}-4-pyridinyl)-1H-benzimidazol-1-yl]-2-thiophenecarboxamide;
3-{[(1R)-1-(2-chlorophenyl)ethyl]oxy}-5-[5-(2-{[2-(4-morpholinyl)ethyl]amino}-4-pyridinyl)-1H-benzimidazol-1-yl]-2-thiophenecarboxamide;
3-{[(1R)-1-(2-chlorophenyl)ethyl]oxy}-5-[5-(2-{[(2R)-2,3-dihydroxypropyl]amino}-4-pyridinyl)-1H-benzimidazol-1-yl]-2-thiophenecarboxamide;
3-{[(1R)-1-(2-chlorophenyl)ethyl]oxy}-5-(5-{2-[(2-hydroxyethyl)amino]-4-pyridinyl}-1H-benzimidazol-1-yl)-2-thiophenecarboxamide;
2-({4-[1-(5-(Aminocarbonyl)-4-{[(1R)-1-(2-chlorophenyl)ethyl]oxy}-2-thienyl)-1H-benzimidazol-5-yl]-2-pyridinyl}amino)ethyl L-valinate;
5-[5-(2-{[2-(Dimethylamino)ethyl]amino}pyrimidin-4-yl)-1H-benzimidazol-1-yl]-3-({(1R)-1-[2-(trifluoromethyl)phenyl]ethyl}oxy)thiophene-2-carboxamide;
3-{[(1R)-1-(2-chlorophenyl)ethyl]oxy}-5-[5-(2-{[3-(4-methyl-1-piperazinyl)propyl]amino}-4-pyrimidinyl)-1H-benzimidazol-1-yl]-2-thiophenecarboxamide;
3-{[(1R)-1-(2-Chlorophenyl)ethyl]oxy}-5-[5-(2-{[3-(dimethylamino)propyl]amino}-5-pyrimidinyl)-1H-benzimidazol-1-yl]-2-thiophenecarboxamide;
3-{[(1R)-1-(2-Chlorophenyl)ethyl]oxy}-5-[5-(2-{[2-(dimethylamino)ethyl]amino}-5-pyrimidinyl)-1H-benzimidazol-1-yl]-2-thiophenecarboxamide;
3-{[(1R)-1-(2-Chlorophenyl)ethyl]oxy}-5-[5-(2-{[3-(4-methyl-1-piperazinyl)propyl]amino}-5-pyrimidinyl)-1H-benzimidazol-1-yl]-2-thiophenecarboxamide;
3-{[(1R)-1-(2-chlorophenyl)ethyl]oxy}-5-(5-{2-[[2-(dimethylamino)ethyl](methyl)amino]-5-pyrimidinyl}-1H-benzimidazol-1-yl)-2-thiophenecarboxamide;
3-{[(1R)-1-(2-chlorophenyl)ethyl]oxy}-5-[5-(2-{[2-(4-morpholinyl)ethyl]amino}-5-pyrimidinyl)-1H-benzimidazol-1-yl]-2-thiophenecarboxamide;
3-{[(1R)-1-(2-chlorophenyl)ethyl]oxy}-5-[5-(3-{[2-(1-pyrrolidinyl)ethyl]oxy}phenyl)-1H-benzimidazol-1-yl]-2-thiophenecarboxamide;
3-{[(1R)-1-(2-chlorophenyl)ethyl]oxy}-5-(5-{4-[(4-morpholinylacetyl)amino]phenyl}-1H-benzimidazol-1-yl)-2-thiophenecarboxamide;
5-[5-(6-{[2-(dimethylamino)ethyl]amino}-3-pyridinyl)-1H-benzimidazol-1-yl]-3-({(1R)-1-[2-(trifluoromethyl)phenyl]ethyl}oxy)-2-thiophenecarboxamide;
5-[5-(6-{[2-(methylamino)ethyl]amino}-3-pyridinyl)-1H-benzimidazol-1-yl]-3-({(1R)-1-[2-(trifluoromethyl)phenyl]ethyl}oxy)-2-thiophenecarboxamide;
3-{[(1R)-1-(2-chlorophenyl)ethyl]oxy}-5-(5-{6-[[2-(dimethylamino)ethyl](methyl)amino]-3-pyridinyl}-1H-benzimidazol-1-yl)-2-thiophenecarboxamide;
3-{[(1R)-1-(2-chlorophenyl)ethyl]oxy}-5-(5-{6-[(2-hydroxyethyl)amino]-3-pyridinyl}-1H-benzimidazol-1-yl)-2-thiophenecarboxamide;
3-{[(1R)-1-(2-chlorophenyl)ethyl]oxy}-5-[5-(6-{[3-(dimethylamino)propyl]amino}-3-pyridinyl)-1H-benzimidazol-1-yl]-2-thiophenecarboxamide;
3-{[(1R)-1-(2-chlorophenyl)ethyl]oxy}-5-[5-(6-{[2-(dimethylamino)ethyl]amino}-3-pyridinyl)-1H-benzimidazol-1-yl]-2-thiophenecarboxamide;
3-{[(1R)-1-(2-chlorophenyl)ethyl]oxy}-5-[5-(6-{[3-(4-methyl-1-piperazinyl)propyl]amino}-3-pyridinyl)-1H-benzimidazol-1-yl]-2-thiophenecarboxamide;
3-{[(1R)-1-(2-chlorophenyl)ethyl]oxy}-5-[5-(6-{[2-(4-morpholinyl)ethyl]amino}-3-pyridinyl)-1H-benzimidazol-1-yl]-2-thiophenecarboxamide;
3-{[(1R)-1-(2-chlorophenyl)ethyl]oxy}-5-(5-{6-[[3-(dimethylamino)propyl](methyl)amino]-3-pyridinyl}-1H-benzimidazol-1-yl)-2-thiophenecarboxamide; and
3-{[(1R)-1-(2-chlorophenyl)ethyl]oxy}-5-[5-(6-{[3-(4-methyl-1-piperazinyl)propyl]amino}-3-pyridazinyl)-1H-benzimidazol-1-yl]-2-thiophenecarboxamide
and pharmaceutically acceptable salts thereof.

8. A pharmaceutical composition comprising a compound according to claim 1.

9. The pharmaceutical composition according to claim 8 further comprising a pharmaceutically acceptable carrier, diluent or excipient.

10. The pharmaceutical composition according to claim 9 further comprising a chemotherapeutic agent.

11. A method for treating a condition mediated by PLK in a human in need thereof, said method comprising administering to the human a therapeutically effective amount of a compound according to claim 1.

12. A method for treating a susceptible neoplasm in a human in need thereof, said method comprising administering to the human a therapeutically effective amount of a compound according to claim 1.

13. The method according to claim 12, wherein said susceptible neoplasm is selected from the group consisting of breast cancer, colon cancer, small cell lung cancer, non-small cell lung cancer, prostate cancer, endometrial cancer, gastric cancer, melanoma, pancreatic cancer, ovarian cancer, squamous cell carcinoma, carcinoma of the head and neck, esophageal carcinoma, hepatocellular carcinoma and hematologic maliginancies.

14. A method for treating a condition characterized by inappropriate cellular proliferation in a human in need thereof, said method comprising administering to the human a therapeutically effective amount of a compound according to claim 1.

15. A process for preparing a compound according to claim 1 wherein Ring A is at the 5-position (I-5), said process comprising either:
Process A:
reacting the compound of formula (VII-5): 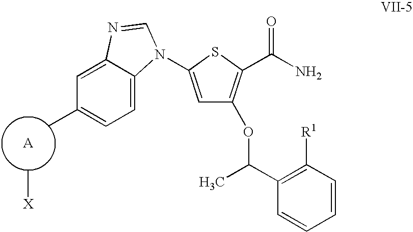
wherein Me is methyl, X is a halogen, and all other variables are as defined in claim 1;
with a compound of formula (H—Y—R²—R³) to prepare a compound of formula (I-5): 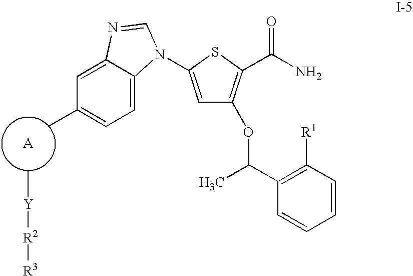
wherein all variables are as defined in claim 1; or
Process B:
reacting a compound of formula (VI-5): 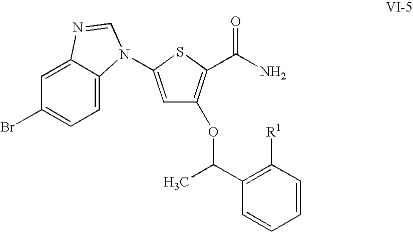
wherein all variables are as defined in claim 1
with an aryl metal reagent of formula (VIII-A): 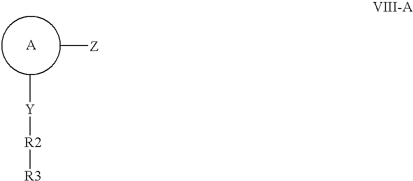
wherein Z is a a boronic acid, boronic ester, trifluoroborate salt, stannane or zinc halide, and all other variables are as defined in claim 1;
to prepare a compound of formula (I-5).

16. A process for preparing a compound according to claim 1, said process comprising either:
Process A:
reacting a compound of formula (VII-5,6): 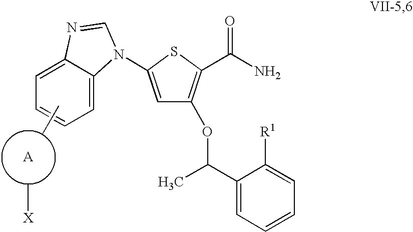
wherein X is halogen and all other variables are as defined in claim 1 and Ring A is bound to the 5- or 6-position of the benzimidazole;
with a compound of formula (H—Y—R²—R³) to prepare a compound of formula (I); or
Process B:
reacting a compound of formula (VI-5,6): 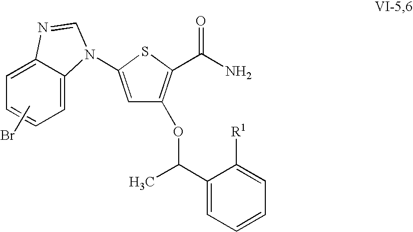
wherein all variables are as defined in claim 1 and Br is bound to the 5- or 6-position of the benzimidazole;
with an aryl metal reagent of formula (VIII-A): 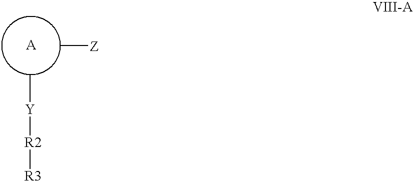
wherein Z is a a boronic acid, boronic ester, trifluoroborate salt, stannane or zinc halide, and all other variables are as defined above;
to prepare a compound of formula (I).

17-24. (canceled)
